(19) United States
(12) Patent Application Publication
(10) Pub. No.: US 2022/0380785 A1
(43) Pub. Date: Dec. 1, 2022

(54) COMPOSITIONS AND METHODS FOR SIALYLATED MUCIN-TYPE O-GLYCOSYLATION OF THERAPEUTIC PROTEINS

(71) Applicant: THE UNIVERSITY OF BRITISH COLUMBIA, Vancouver (CA)

(72) Inventors: Stephen G. WITHERS, Vancouver (CA); Lyann SIM, Vancouver (CA); Warren William WAKARCHUK, Edmonton (CA)

(73) Assignee: THE UNIVERSITY OF BRITISH COLUMBIA, Vancouver, BC (CA)

(21) Appl. No.: 17/773,543

(22) PCT Filed: Nov. 1, 2020
(86) PCT No.: PCT/CA2020/051480

§ 371 Date: Apr. 29, 2022

Related U.S. Application Data
(60) Provisional application No. 62/929,733, filed on Nov. 1, 2019.

(51) Int. Cl. C12N 15/70; C12P 21/00; C12N 9/10; C12N 9/90

(52) U.S. Cl. CPC C12N 15/70; C12P 21/005; C12N 9/1051; C12N 9/1081; C12N 9/90; C12Y 204/01041; C12Y 204/99001; C12Y 204/99004

(57) ABSTRACT

Provided herein are enzymatic compositions for protein O-glycosylation and sialylation, methods and systems associated therewith. In particular, the composition for in vivo sialylation of therapeutic proteins. The composition comprises a polypeptide N-acetylgalactosaminyltransferase; a β-1,3-galactosyltransferase; an UDP-Glc/GlcNAc 4-epimerase; a disulfide bond isomerase; and an α-2,3-sialyltransferase or an α-2,6-sialyltransferase. Furthermore, provided herein are compositions for efficient and complete O-glycosylation and di-sialylation of therapeutic proteins.

### CROSS REFERENCE TO RELATED APPLICATION

This application claims the benefit of U.S. Provisional Patent Application Ser. No. 62/929,733 filed on 1 Nov. 2019, entitled “COMPOSITIONS AND METHODS FOR SIALYLATED MUCIN-TYPE O-GLYCOSYLATION OF THERAPEUTIC PROTEINS”.

### TECHNICAL FIELD

The present invention relates to the field of enzyme compositions. In particular, the invention relates to enzyme compositions for protein O-glycosylation and sialylation, and for providing methods and systems for in vivo protein sialylation of therapeutic proteins using the compositions.

### BACKGROUND

The production of the majority of human therapeutic biologics relies on mammalian cellular hosts (e.g. CHO and HEK cells) with machinery that can properly fold and add human-like post-translational modifications to the target protein. However, disadvantages of mammalian cellular hosts include slow growth, costly media, and the fact that homogenous glycosylation is often difficult to achieve due to competing endogenous glycosyltransferases. There has thus been considerable effort to engineer these strains to improve their performance as well as development other eukaryotic hosts (i.e. yeast, insects, plants) to produce more human-like glycan structures¹.

The use of E. coli as host for production of mammalian proteins has also been reported, but the targets are often produced as insoluble inclusion bodies and must be refolded, usually resulting in low yields². Immense engineering advances have correspondingly been made to render E. coli more suitable for production of biologics, in terms of removing endotoxins, improving protein folding, and introducing glycosylation2. With regards to glycoengineering, the first bacterial glycoprotein platform used the GT66³ family bacterial oligosaccharide transferase PglB to catalyze the transfer of a bacterial oligosaccharide structure en bloc from a lipid-linked oligosaccharide onto an Asn residue of a protein to form an N-glycosylated protein4. However, the efficiency of this periplasmic glycosylation system is very low (<1% protein modified), largely due to the complexity of this systems. With the more recent discoveries of glycosyltransferases that carry out cytoplasmic bacterial glycosylation, effort has been put towards adapting these enzymes for glycosylation of non-native recombinant targets within E. coli. In one case E. coli expression of an Actinobacillus pleuropneumoniae N-glucosyltransferase, from the GT41 family enabled transfers of glucose onto an Asn in the normal GT66 oligosaccharyltransferase sequon of Asn-X-Ser/Thr⁶. Although this particular modification is not found in humans, it has the potential to be used as a handle for further modifications, such as polysialylation⁵. However, efficient and complete sialylation of human proteins expressed in prokaryotic systems has remained elusive.

### SUMMARY

The present invention is based in part, on the surprising discovery that particular combinations of enzymes are able to completely monosialylate and disialylate therapeutic human proteins in a bacterial cell. Furthermore, it was surprisingly discovered that mammalian sialyltransferases pST3Gal1, hST6GalNAc2 and hST6GalNAC4 could be used to sialylate protein targets in vivo in the E. coli host cytoplasm in combination with a prokaryotic β1,3-galactosyltransferase, Cgtb. Alternatively, a Drosophila melanogaster C1GalT1 galactosyltransferase (DmC1GalT1) may be used.

In accordance with one embodiment, there is provided a plasmid, the plasmid may include DNA encoding: (a) a polypeptide N-acetylgalactosaminyltransferase; (b) a β-1,3-galactosyltransferase; (c) an UDP-Glc/GlcNAc4-epimerase; (d) a disulfide bond isomerase; (e) an α-2,3-sialyltransferase; and (f) an α-2,6-sialyltransferase.

In accordance with a further embodiment, there is provided a recombinant bacterial cell, the bacterial cell having an oxidizing environment and including a plasmid or plasmids described herein.

In accordance with a further embodiment, there is provided a recombinant bacterial cell, wherein said bacterial cell provides an oxidizing environment and includes a chromosome, wherein the chromosome may include integrated DNA encoding: (a) a polypeptide N-acetylgalactosaminyltransferase; (b) a β-1,3-galactosyltransferase; (c) an UDP-Glc/GlcNAc 4-epimerase; (d) a disulfide bond isomerase; (e) an α-2,3-sialyltransferase; and (f) an α-2,6-sialyltransferase.

In accordance with a further embodiment, there is provided a nucleic acid construct that directs expression in a prokaryotic cell, the nucleic acid construct may include DNA encoding: (a) a polypeptide N-acetylgalactosaminyltransferase; (b) a β-1,3-galactosyltransferase; (c) an UDP-Glc/GlcNAc 4-epimerase; (d) a disulfide bond isomerase; (e) an α-2,3-sialyltransferase; (f) an α-2,6-sialyltransferase; and (g) at least one promoter.

In accordance with a further embodiment, there is provided a method for producing a sialylated or disialylated target protein in a bacterium, wherein the bacterium provides an oxidizing environment for posttranslational modification of expressed protein, the method including: (a) expressing in the bacterium: a polypeptide N-acetylgalactosaminyltransferase; a β-1,3-galactosyltransferase; an UDP-Glc/GlcNAc 4-epimerase; a disulfide bond isomerase; an α-2,3-sialyltransferase; an α-2,6-sialyltransferase (b) expressing in the bacterium: a hydrolysing UDP-GlcNAc 2′ epimerase; a sialic acid synthetase; and a CMP-NeuAc synthetase; and (c) expressing in the bacterium a target protein for O-glycosylation and sialylation or disialylation.

The plasmid may include at least 2 operons, wherein the DNA encoded in operon 1 includes: (i) at least 1 promoter; (ii) a polypeptide N-acetylgalactosaminyltransferase; (iii) a disulfide bond isomerase; and (iv) an UDP-Glc/GlcNAc 4-epimerase; and wherein the DNA encoded in operon 2 may include: (v) at least 1 promoter; (vi) a β-1,3-galactosyltransferase; (vii) anα-2,3-sialyltransferase; and (viii) anα-2,6-sialyltransferase.

The plasmid may include at least 2 operons, wherein the DNA encoded in operon 1 may include: (i) at least 1 promoter; (ii) a polypeptide N-acetylgalactosaminyltransferase; (iii) a disulfide bond isomerase; (iv) an UDP-Glc/GlcNAc 4-epimerase; and (v) anα-2,6-sialyltransferase; and wherein the DNA encoded in operon 2 may include: (vi) at least 1 promoter; (vii) a β-1,3-galactosyltransferase; and (viii) anα-2,3-sialyltransferase.

The plasmid may further include a ribosomal binding site encoded upstream of the start codon of each encoded gene. The promoter in operon 1 and operon 2 may be selected from: an inducible promoter and a constitutive promoter. The plasmid may have three copies of the promoter in operon 1 and one copy of the promoter in operon 2.

The polypeptide N-acetylgalactosaminyltransferase may be human polypeptide N-acetylgalactosaminyltransferase 2 (hppGalNAcT2)—(SEQ ID NO:1) or (any one of SEQ ID NO:1-20). The β-1,3-galactosyltransferase may be Campylobacter jejuni β-1,3-galactosyltransferase (CgtB) (SEQ ID NO:21) or (any one of SEQ ID NO:21-24) or Drosophila melanogaster C1GalT1 galactosyltransferase (DmC1GalT1) (SEQ ID NO:22 or 23) or (any one of SEQ ID NO:21-24). The UDP-Glc/GlcNAc 4-epimerase may be Campylobacter jejuni UDP-Glc/GlcNAc 4-epimerase (Cj-Gne) (SEQ ID NO:25) or (any one of SEQ ID NO:25-29). The disulfide bond isomerase may be selected from: human disulfide bond isomerase (hPDI) (SEQ ID NO:36) or (any one of SEQ ID NO:30-36); and E. coli disulfide bond isomerase (DsbC) (SEQ ID NO:30) or (any one of SEQ ID NO:30-36). The α-2,3-sialyltransferase (any one of SEQ ID NO: 37-42) may be selected from: Campylobacter jejuni α2,3-sialyltransferase (CST-I) (SEQ ID NO:37 or 39); and porcine ST3Gal1 (pST3Gal1) (SEQ ID NO:38). The α-2,3-sialyltransferase may be selected from: Campylobacter jejuni α2,3-sialyltransferase (CST-I) (SEQ ID NO: 37 or 39); Campylobacter jejuni α2,3-sialyltransferase (CST-II) (SEQ ID NO:40 or 42); and porcine ST3Gal1 (pST3Gal1) (SEQ ID NO:38). Alternatively, the α-2,3-sialyltransferase may be Campylobacter jejuni α2,3-sialyltransferase (CST-II) UniProtKB—Q9LAK3—SEQ ID NO:41 (Q9LAK3_CAMJU). The α-2,6-sialyltransferase may be selected from: hST6GalNAc2 (SEQ ID NO:43); and hST6GalNAc4 (SEQ ID NO:44) (or any one of SEQ ID NO:43-52). Alternatively, the α-2,3-sialyltransferase may be Campylobacter jejuni α2,3-sialyltransferase (CST-II) (SEQ ID NO:40 or 42).

The plasmid may encode enzymes including an amino acid sequences as set forth in one or more of SEQ ID NO: 1-52, or an amino acid sequence having at least 90% sequence identity thereto, provided that the enzymes retain their enzymatic activity.

The plasmid may have the DNA sequence set out in one of SEQ ID NO: 60-62, or a nucleic acid sequence having at least 90% sequence identity thereto, provided that the enzymes encoded by the sequence retain their enzymatic activity.

The plasmid may further include a second plasmid, wherein the second plasmid may include a DNA sequence encoding: (a) a hydrolysing UDP-GlcNAc 2′ epimerase; (b) a sialic acid synthetase; and (c) a CMP-NeuAc synthetase.

The second plasmid may include at least 1 operon, wherein the DNA encoded in operon 3 may include: (i) at least 1 promoter; (ii) a hydrolysing UDP-GlcNAc 2′ epimerase; (iii) a sialic acid synthetase; and (iv) a CMP-NeuAc synthetase.

The plasmid may further include a ribosomal binding site encoded upstream of the start codon of each encoded gene. The promoter in operon 3 may be selected from: an inducible promoter and a constitutive promoter. There may be three copies of the promoter in operon 3. Operon 3 may be a Neisseria meningitidis neuBCA operon. The plasmid may encode enzymes including an amino acid sequence or sequences as set forth as SEQ ID NO: 16-18, or an amino acid sequence having at least90% sequence identity thereto, provided that the enzymes retain their enzymatic activity. The plasmid may have the DNA sequence set out in SEQ ID NO: 63, or a nucleic acid sequence having at least 90% sequence identity thereto, provided that the enzymes encoded by the sequence retain their enzymatic activity.

The plasmid may further include a third plasmid, wherein the third plasmid may include a DNA sequence encoding a target gene for expression, O-glycosylation, and sialylation or disialylation.

The bacterial cell may expresses the integrated DNA encoding (a)-(f) under the control of at least 1 promoter. The bacterial cell may further include a ribosomal binding site encoded upstream of the start codon of each encoded gene. The at least one promoter may be selected from: an inducible promoter and a constitutive promoter. The chromosome may encode enzymes may include an amino acid sequence as set forth in one or more of SEQ ID NOs:1-52, or an amino acid sequence having at least 90% sequence identity thereto, provided that the enzymes retain their enzymatic activity. The chromosome may have a DNA sequence set out in SEQ ID NOs:60-62, or a nucleic acid sequence having at least 90% sequence identity thereto, provided that the enzymes encoded by the sequences retain their enzymatic activity.

The bacterial cell chromosome may further include integrated DNA encoding: (g) a hydrolysing UDP-GlcNAc 2′ epimerase; (h) a sialic acid synthetase; and (i) a CMP-NeuAc synthetase.

The bacterial cell may express the further integrated DNA encoding (g)-(i) under the control of at least 1 promoter. The bacterial cell may further include a ribosomal binding site encoded upstream of the start codon of each encoded gene. The bacterial cell may have at least one promoter selected from: an inducible promoter and a constitutive promoter. The bacterial cell operon 3 may be a Neisseria meningitidis neuBCA operon.

The bacterial cell chromosome may encode enzymes including an amino acid sequence as set forth in one or more of SEQ ID NOs: 1-52, or an amino acid sequence having at least90% sequence identity thereto, provided that the enzymes retain their enzymatic activity. The bacterial cell chromosome may have the DNA sequence set out in SEQ ID NO: 63, or a nucleic acid sequence having at least 90% sequence identity thereto, provided that the enzymes encoded by the sequences retain their enzymatic activity.

The bacterial cell may further include a DNA sequence encoding a target gene for expression, O-glycosylation, and sialylation or disialylation. The bacterial cell may be modified to reduce reductase activity.

The nucleic acid construct may further include a ribosomal binding site encoded upstream of the start codon of each encoded gene. The nucleic acid construct may include at least one promoter which may be selected from: an inducible promoter and a constitutive promoter.

The nucleic acid construct may encode enzymes including an amino acid sequence as set forth in one or more of SEQ ID NO: 1-52, or an amino acid sequence having at least90% sequence identity thereto, provided that the enzymes retain their enzymatic activity. The nucleic acid construct may have the DNA sequence set out in SEQ ID NO: 60-62, or a nucleic acid sequence having at least90% sequence identity thereto, provided that the enzymes encoded by the sequences retain their enzymatic activity.

The nucleic acid construct may further include DNA encoding: (h) at least one promoter; (i) a hydrolysing UDP-GlcNAc 2′ epimerase; (j) a sialic acid synthetase; and (k) a CMP-NeuAc synthetase.

The nucleic acid construct may further include a ribosomal binding site encoded upstream of the start codon of each encoded gene. The nucleic acid construct promoter may be selected from: an inducible promoter and a constitutive promoter. The nucleic acid construct having the hydrolysing UDP-GlcNAc 2′ epimerase; the sialic acid synthetase; and the CMP-NeuAc synthetase may be a Neisseria meningitidis neuBCA operon.

The nucleic acid construct may encode enzymes may include an amino acid sequence as set forth as SEQ ID NO: 53-55, or an amino acid sequence having at least 90% sequence identity thereto, provided that the enzymes retain their enzymatic activity. The nucleic acid construct may have the DNA sequence set out in SEQ ID NO: 63, or a nucleic acid sequence having at least 90% sequence identity thereto, provided that the enzymes encoded by the sequences retain their enzymatic activity. The nucleic acid construct may further include DNA encoding a target gene for expression, O-glycosylation, and sialylation or disialylation. The nucleic acid construct described herein may reside in a bacterial cell. The bacterial cell may be modified to provide an oxidizing environment within the cell cytoplasm. The bacterial cell may be modified to reduce reductase activity.

Alternatively, there may be 90% sequence identity as defined by NCBI BLAST sequence similarity search tool, using the default settings for nucleotide searching or protein searching. Alternatively, there may be 90% sequence similarity as defined by NCBI BLAST sequence similarity search tool, using the default settings for nucleotide searching or protein searching. The sequence similarity may be at least 91%. The sequence similarity may be at least 92%. The sequence similarity may be at least 93%. The sequence similarity may be at least 94%. The sequence similarity may be at least 95%. The sequence similarity may be at least 96%. The sequence similarity may be at least 97%. The sequence similarity may be at least 98%. The sequence similarity may be at least 99%. Alternatively, the sequence identity may be at least 91%. The sequence identity may be at least 92%. The sequence identity may be at least 93%. The sequence identity may be at least 94%. The sequence identity may be at least 95%. The sequence identity may be at least 96%. The sequence identity may be at least 97%. The sequence identity may be at least 98%. The sequence identity may be at least 99%. The sequence similarity or the sequence identity may be determined by NCBI BLAST sequence similarity search tool, using the default settings for nucleotide searching or protein searching.

Furthermore, it will be appreciated by a person of skill in the art, that the enzymes described herein and the target protein may be expressed in plasmids, integrated into the bacterial chromosome or some combination of both.

The sequences described herein may include whatever promoters, cofactors, ribosomal binding sites etc. as may be required to effectively transcribe, translate and post-translationally modify the target proteins and to do so with a high degree of efficiency and complete O-glycosylation and sialylation of target proteins.

### BRIEF DESCRIPTION OF THE DRAWINGS

FIG. 1 shows a schematic of an exemplary protein O-glycosylation and CMP-Neu5Ac synthesis in engineered Origami 2™ (DE3)neu⁺E. coll. O-Glycosylation of the target protein is initiated by ppGalNAcT, followed by galactosylation by CgtB and sialylation (bacterial or mammalian α-2,3- and 2,6-sialylatransferases, STs), wherein enzymes encoded on O-Glycosylation Operons (OGO) operons are ppGalNAcT2; CgtB; STs; Gne and the neuB/C/A genes are been integrated into the Origami 2™ or OG2 (DE3) genome.

FIG. 2 shows the detection of CMP-Sia in OG2neu+engineered strain with CST-I, where the presence of CMP-Sia in lysate (from OG2 (lane3) and OG2neu⁺+/−IPTG induced cells (lanes 4 and 5)) was detected on TLC by CST-I conversion of BODIPY-Lac to BODIPY-SiaLac.

FIG. 3 shows a schematic representation of sialylation operons tested: A) original OGO-1 and OGO-2 operons described in Du et al.⁸ are shown; B) mono-sialylation operons incorporating either bacterial CST-I or mammalian pST3Gal1 α-2,3-sialyltransferases (see OGO-3; OGO-4; OGO-5; OGO-6; and OGO-11); and C) disialylation operon variations of OGO-5 containing hST6GalNAc2 or hST6GalNAc4 (see OGO-7; OGO-8; and OGO-9), with the exception of OGO-10, which has a second α-2,3-sialyltransferase.

FIG. 4 shows SiaTAg modification of GB1-IFNα2b by intact mass analysis of GB1-IFNα2b after co-expression with: A) OGO-1 (control); B) OGO-4 (CST-I); and C) OGO-5 (pST3Gal1).

FIG. 5 shows a comparison of glycosylation on GB1-IFNα2b* and GB1-hGH*, by intact mass analysis of GB1-IFNα2b* from A)OGO-5 and B)OGO-8 and GB1-hGH* from C)OGO-5 and D)OGO-8 co-expression trials.

FIG. 6 shows Core-GalNAc sialylation on GB1-IFNα2b using ST6GalNAc2 with intact mass analysis of GB1-IFNα2b from co-expression with disialylation plasmid OGO-9.

FIG. 7 shows an Intact mass of GB1-IFNα2b* from co-expression trials of OGO-8 with QSox1b in the JM109(DE3) neu+strain.

FIG. 8 shows plasmid maps of OGO-7, OGO-8, OGO-9 and OGO-10.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

The present application is directed toward developing an E. coli expression system that is capable of producing proteins modified with human-like sialylated mucin-type O-glycans. The inventors were successful at developing a platform that could produce human cytokines modified with a mixture of Tn-antigen (Tn, GalNAcα) and T-antigen (T-Ag, Gal-β1,3-GalNAcα)⁸. This was achieved using a two-plasmid approach wherein human polypeptide GalNAc transferase 2 (hppGalNAcT2), Campylobacter jejuni β1,3-galactosyltransferase (CgtB) and UDP-Glc/GlcNAc 4-epimerase (gne), and either human or E. coli disulfide bond isomerase (hPDI or DsbC, respectively) were expressed on an operon encoded by one plasmid, and the target protein on another. With this approach, they achieved up to 100 mg/mL of target protein production with 85% glycosylation. This work sets the stage for further T-Ag modification, including sialylation, which has been shown to be crucial for improved pharmacokinetic properties of therapeutic proteins^{9,10}. Indeed, disialyl-TAg—modified EPO-Fc fusion has reportedly been made in engineered N. benthamiana through the introduction of mammalian glycosyltransferases, but not in prokaryotic systems¹¹. A major challenge has been that the expression of mammalian STs within E. coli typically results in misfolded proteins. However, specialized strains of E. coli with oxidizing environments (i.e. Origami 2™ and Shuffle™ strain)¹² have been shown to support production of selected mammalian sialyltransferases including porcine ST3Gal1 (pST3Gal1) and human ST6Gal1, opening up options^{13,14}. However, the use of a nucleic acid construct that is integrated into the bacterial chromosome could also be used to provide a more stable expression system.

The goal of the inventors was to extend the core T-Ag structure to produce proteins decorated with mono and di-sialylated T-Ag. Towards this aim, we have engineered our host E. coli strain to produce the CMP-Neu5Ac donor and constructed new OGO operons incorporating either bacterial or mammalian sialyltransferases. The in vivo sialylation of a GB1-fusion human Interferon-α2b (GB1-IFnα2b)⁸ cytokine produced by co-expression of these new operons is evaluated herein.

Any terms not directly defined herein shall be understood to have the meanings commonly associated with them as understood within the art of the invention.

As used herein, “target proteins” are meant to include any protein that would benefit from O-glycosylation and sialylation. These may be therapeutic proteins.

As used herein “polypeptide N-acetylgalactosaminyltransferase” (EC 2.4.1.41) refers to an enzyme that catalyzes the chemical reactions:
(1) UDP-N-acetyl-alpha-D-galactosamine+[protein]-L-serine<=>UDP+[protein]-3-O-(N-acetyl-alpha-D-galactosaminyl)-L-serine; and
(2) UDP-N-acetyl-alpha-D-galactosamine+[protein]-L-threonine<=>UDP+[protein]-3-O-(N-acetyl-alpha-D-galactosaminyl)-L-threonine.
   For the most part, any mammalian polypeptide N-acetylgalactosaminyltransferase would work. For example, a polypeptide N-acetylgalactosaminyltransferase may be selected from human polypeptide N-acetylgalactosaminyltransferase 2 (hppGalNAcT2—SEQ ID NO:1 (UniProtKB—Q10471, GALT2_HUMAN—SEQ ID NO:2); human GALT1 (Q10472)—SEQ ID NO:3, GALT3 (Q14435)—SEQ ID NO:4, GALT4 (Q8N4A0)—SEQ ID NO:5, GALT5 (Q7Z7M9)—SEQ ID NO:6, GALT6 (Q8NCL4)—SEQ ID NO:7, GALT7 (Q86SF2)—SEQ ID NO:8, GALT8 (Q9NY28)—SEQ ID NO:9, GALT9 (Q9HCQ5)—SEQ ID NO:10, GALT10 (Q86SR1)—SEQ ID NO:11, GALT11 (Q8NCW6)—SEQ ID NO:12, GALT12 (Q8IXK2)—SEQ ID NO:13, GALT13 (Q8IUC8)—SEQ ID NO:14, GALT14 (Q96FL9)—SEQ ID NO:15, GALT15 (Q8N3T1)—SEQ ID NO:16, GALT16, (Q8N428)—SEQ ID NO:17, GALT17 (Q6IS24)—SEQ ID NO:18, GALT18(Q6P9A2)—SEQ ID NO:19) or any ppGalNAcT 1 to 20 enzymes. Also, GLTL6, which is polypeptide N-acetylgalactosaminyltransferase-like 6 ((Q49A17)—SEQ ID NO:20).

As used herein “β-1,3-galactosyltransferase” refers to an enzyme that that transfers galactose from UDP-alpha-D-galactose to substrates with a terminal beta-N-acetylglucosamine (beta-GlcNAc) residue. As exemplified herein, the β-1,3-galactosyltransferase may be Campylobacter jejuni β-1,3-galactosyltransferase (CgtB)—SEQ ID NO:21 or a Drosophila melanogaster C1GalT1 galactosyltransferase (DmC1GalT1)—SEQ ID NO:22 and 23. For example, β-1,3-galactosyltransferase may be selected from Drosophila melanogaster core 1 galactosyltransferase A (AAF52723) or Campylobacter jejuni β-1,3-galactosyltransferase (CgtB)—SEQ ID NO:21 or Drosophila melanogaster C1GalT1 galactosyltransferase (DmC1GalT1)—SEQ ID NO:22 and 23, for example, UniProtKB—Q7K237—SEQ ID NO:22. Alternatively, the β-1,3-galactosyltransferase may be represented by GALT_HUMAN Galactose-1-phosphate uridylyltransferase (UniProtKB—P07902)—SEQ ID NO:24 (previously WP_052404662.1).

As used herein “UDP-Glc/GlcNAc 4-epimerase” refers to an enzyme that catalyzes two reactions: the reversible epimerization of UDP-glucose to UDP-galactose and the reversible epimerization of UDP-N-acetylglucosamine to UDP-N-acetylgalactosamine. For example, the UDP-Glc/GlcNAc 4-epimerase may be selected from UniProtKB—Q0P9C3 (Q0P9C3_CAMJE)—SEQ ID NO:26 which replaces A0A0M5MRS0 (A0A0M5MRS0_CAMJU); Q14376—SEQ ID NO:27 (GALE_HUMAN); Q8R059—SEQ ID NO:28 (GALE_MOUSE); and P09147—SEQ ID NO:29 (GALE_ECOLI). The UDP-Glc/GlcNAc 4-epimerase may be Campylobacter jejuni UDP-Glc/GlcNAc 4-epimerase (Cj-Gne)—SEQ ID NO:25.

As used herein “disulfide bond isomerase” refers to an enzyme that catalyzes the rearrangement of -S-S- bonds in proteins. For example, the disulfide bond isomerase, may be selected from UniProtKB—Q14554—SEQ ID NO:31 (PDIA5_HUMAN); UniProtKB—P30101—SEQ ID NO:32 (PDIA3_HUMAN); UniProtKB—P07237—SEQ ID NO:33 (PDIA1_HUMAN); UniProtKB—P13667—SEQ ID NO:34 (PDIA4_HUMAN); and UniProtKB—P0AEG6—SEQ ID NO:35 (DSBC_ECOLI). The disulfide bond isomerase may be selected from: human disulfide bond isomerase (hPDI)—SEQ ID NO:36; or E. coli disulfide bond isomerase (DsbC)—SEQ ID NO:30.

As used herein “α-2,3-sialyltransferase” refers to an enzyme that catalyzes the synthesis of the sequence NeuAc-alpha-2,3-Gal-beta-1,3-GalNAc—found on sugar chains O-linked to Thr or Ser and also as a terminal sequence on certain gangliosides. For example, the α-2,3-sialyltransferase may be selected from ST3Gal I, UniProtKB—Q9RGF1—SEQ ID NO:39 (Q9RGF1_CAMJU); and UniProtKB—Q11201—SEQ ID NO:40 (SIA4A_HUMAN). The α-2,3-sialyltransferase may be selected from: Campylobacter jejuni α2,3-sialyltransferase (CST-I)—SEQ ID NO:37; and porcine ST3Gal1 (pST3Gal1) -SEQ ID NO:38. Alternatively, the α-2,3-sialyltransferase may be Campylobacter jejuni α2,3-sialyltransferase (CST-II)—SEQ ID NO:42 or UniProtKB—Q9LAK3—SEQ ID NO:41 (Q9LAK3_CAMJU).

As used herein “α-2,6-sialyltransferase” refers to an enzyme that catalyzes the transfer of N-acetylneuraminyl groups onto glycan chains in glycoproteins. For example, the α-2,6-sialyltransferase may be selected from ST6GalNAc I, II, III or IV, UniProtKB—Q9UJ37—SEQ ID NO:45 (SIA7B_HUMAN); UniProtKB—Q9H4F1—SEQ ID NO:46 (SIA7D_HUMAN); UniProtKB—Q9BVH7—SEQ ID NO:47 (SIA7E_HUMAN); UniProtKB—Q9NSC7—SEQ ID NO:48 (SIA7A_HUMAN); UniProtKB—Q8NDV1—SEQ ID NO:49 (SIA7C_HUMAN); UniProtKB—Q969X2—SEQ ID NO:50 (SIA7F_HUMAN); UniProtKB—Q96JF0—SEQ ID NO:51 (SIAT2—HUMAN); and UniProtKB—P15907—SEQ ID NO:52 (SIAT1_HUMAN). The α-2,6-sialyltransferase may be selected from: hST6GalNAc2—SEQ ID NO:43; and hST6GalNAc4—SEQ ID NO:44.

The “neuBCA operon” as described herein may be obtained from bacterial strains that are able to make sialic acid de novo such E. coli K1, Neisseria meningitidis and Campylobacter jejuni. The neuBCA operon (SEQ ID NO:63) encodes three enzymes that convert the existing intracellular pool of UDP-GlcNAc (normally used for cell wall biosynthesis) into CMP-NeuAc. NeuC is a hydrolysing UDP-GlcNAc 2′ epimerase (SEQ ID NO:53), which generates free ManNAc, neuB is a sialic acid synthetase (SEQ ID NO:55), condensing ManNAc with pyruvic acid, and neuA is a CMP-NeuAc synthetase (SEQ ID NO:54), coupling sialic acid to CMP. The enzymes may be from Neisseria meningitides.

As used herein “plasmid” refers to a DNA molecule usually between 1 to over 200 kbp within a cell, is separate from chromosomal DNA and is able to replicate independently of a cell chromosome(s). Plasmids usually are circular double-stranded DNA molecules found in bacteria, but may also be found in archaea and in some eukaryotic cells. In molecular biology, plasmids most commonly serve as vectors to express recombinant DNA sequences in a host bacteria. In contrast to viruses, plasmids lack a protective protein coat and only sometimes encode genes needed for their own transfer to another cell. The number of identical plasmids in a single cell can range anywhere from one to thousands.

As used herein “operon” refers to a functional unit of DNA containing a group of genes under the control of a single promoter, that are transcribed together into an mRNA strand and either translated together in the cytoplasm or splicing to create monocistronic mRNAs that are translated separately, i.e. several strands of mRNA that each encode a single gene product. The result of this is that the genes contained in the operon are either expressed together or not at all. Several genes must be co-transcribed to define an operon.

As used herein “promoter” refers to a region or regions of DNA that provide binding sites for RNA polymerase and transcription factors that initiate of transcription of a particular gene. Promoters are located towards the 5′ end of the sense strand near the transcription start site of a gene and are usually about 100-1000 bp. In bacteria, the promoter often contains two short sequence elements approximately 10 and 35 nucleotides upstream from the transcription start site (i.e. −10 consensus sequence TATAAT; and −35 consensus sequence TTGACA). As used herein, the term promoter may also include an “operator” as in the lac promoter and tac promoter systems. The lac promoter is Isopropyl β-d-1-thiogalactopyranoside (IPTG) inducible, whereby in addition to the lac promoter, the lac operator is also needed. If the lac operator were not present the IPTG would not have an inducible effect. As described herein, the Tac-Promoter system (Ptac) is referred to herein as a tac promoter, while in fact tac is both a promoter and an operator. Ptac is an engineered DNA promoter commonly used for protein production in Escherichia coli. Ptac was resulted from a combination of promoters from the trp and lac operons.

As used herein “inducible promoter” refers to a system whereby the expression of a gene or genes is operably linked to either a chemically and physiologically induced promoter to control the expression of a gene or genes of interest. Inducible promoters were developed to limit the synthesis of a gene product or products precisely defined conditions and/or to within a certain time interval. Ideally, an inducible promoter has a strong effect on the gene expression and can be quickly turned on or off as needed in a cost-efficient manner. Furthermore, if the protein or proteins being expressed are toxic to the cell it is important to limit their production to avoid limiting production quality and/or quantity. Chemically induced systems are regulated by the presence or absence of chemical compounds (i.e. alcohols, antibiotics, carbon source, hormones etc.), while physiologically induced promoters are regulated by factors such as osmotic stress, temperature, light, etc.

As used herein “constitutive promoter” refers to an unregulated promoter that allows for continual transcription of the associated gene or genes.

As used herein “ribosomal binding site” refers to a particular consensus sequence in bacterial and archaeal messenger RNA (mRNA), which acts to recruit the ribosome to the mRNA to initiate protein synthesis by aligning the ribosome with the start codon (i.e. AUG) often (i.e. Shine-Dalgarno (SD) sequence). The tRNA then adds amino acids in sequence as dictated by the codons of the gene, moving downstream from the translational start site. Accordingly, the ribosomal binding site is usually located about 8 bases upstream of the start codon. Once recruited, tRNA may add amino acids in sequence as dictated by the codons, moving downstream from the translational start site. In Escherichia coli, the six-base consensus sequence is AGGAGG.

As used herein an enzyme in an “oxidized state” refers to an enzyme that has fewer electrons than its reduced form. As used herein a “reducing agent” refers to a compound that acts by donating electrons (i.e. by becoming oxidized itself).

As used herein “oxidizing environment” refers to an environment in which a substrate is more likely to become oxidized than reduced. For example, wherein a bacteria has a mutation in an endogenous reductase nucleic acid to reduce production of the reductase such that the normally reducing environment of a wild type E. coli cell is oxidizing.

As used herein “nucleic acid construct” refers to a recombinant nucleic acid construct that comprises a segment of nucleic acids for transplantation into a target tissue or cell. The nucleic acid is usually DNA, but in the case of some viruses may be RNA. A nucleic acid construct may contain a gene sequence or the sequences of numerous genes encoding a protein or proteins of interest, may or may not be sub-cloned into a vector, and may contain bacterial resistance genes for growth in bacteria, a ribosomal binding site and one or more promoters for expression in the host organism.

Various alternative embodiments and examples are described herein. These embodiments and examples are illustrative and should not be construed as limiting the scope of the invention.

### Materials and Methods

### Plasmids and Cloning

The GB1-IFNα2b expression vector (pET21b-GB1-IFNα2b) is described⁸.

E. coli codon optimized genes for human ST6GalNAc2 (aa 52-374) and human ST6GalNAc4 (aa 34-302) were ordered and subcloned into the pMal-c5x vector (NEB) in the Ndel/SalI restriction enzyme sites to form HUST-95 and HUST-108, respectively. A dual-promoter expression vector encompassing GB1-IFNα2b and hST6GalNAc2 (pETd-IFN+6A2) was constructed by sequential subcloning of hST6GalNAc2 from HUST-95 (using Ndel and SalI enzymes) and GB1-IFNα2b from HTP-38 (using Xbal and SalI enzymes) into the Ndel/Xhol and XbaI/SalI restriction sites of pETduet-1 (Novagen™), respectively. The pETd-IFN+6A4 dual GB1-IFNα2b+hST6GalNAc4 expression vector was constructed similarly as described for pETd-IFN+6A2. However, with hST6GalNac4 extracted from HUST-108 (using Ndel and SalI).

Shuttle vectors pCW-CgtBS42-CstI and pMal-c5x-pST3Gal1-CgtBS42 containing bi-cistronic assemblies of sialyltransferase (either CST-1 or pST3Gal) and CgtB* were made prior to construction of OGO operons.

pCW-CgTBS42-CstI was used in preparation for cloning of OGO-4. Genes were assembled via a 3-part ligation with a pCWori+vector backbone²³ (digested with Ndel/SalI). CgtBS42 was obtained by PCR amplified product of pCW-CgtBS42-MBP 18 digested with NdeI/XbaI and CST-I from XbaI/SalI digest of pET28a-CST95²⁴. The RBS and His-tag upstream of CST-I were originally transferred from the pET28a vector, however, the latter was removed by mutagenesis with the LS24_F/LS24R primers. Sequencing showed that instead of the L227G mutation contained in CgtB-S42 enhanced mutant¹⁸, this clone contains an L227R mutation. This mutation was eventually traced back to the pCW-CgtBS42-MBP plasmid. For the purpose of this study, it is now referred to as an enhanced CgtB* variant.

The pMal-c5x-pST3Gal1-CgtBS42 plasmid used for assembly of OGO-3 and was constructed via 3-part Gibson assembly with PCR products from amplified pMal-c5x backbone (primers LS19-VF/LS19-VR), pST3Gal1¹³, primers LS19-1F/ LS19-1R), and CgtBS42 (from CJL-223, primers LS19-2F/LS19-2R).

### Construction of Sialylation Operons

OGO-3 (SEQ ID NO:56) was constructed via a 3-part Gibson Assembly™ using a portion of the OGO-1 plasmid (including vector backbone, hppGalNAcT2 and gne) (amplified with primers LS25-VF/LS25-VaR), DsbC gene with upstream RBS site (from OGO-1, primers LS25-IaF/LS25-IaR), and pST3Gal1-CgtBS42 bicistronic gene (from pMal-C5x-pST3Gal1-CgtBS42, primers LS25-IbF/LS25-IR).

The MBP gene upstream of pST3Gal1 in OGO-3 (SEQ ID NO:56), was deleted via PCR, to make the OGO-5 construct (using primers LS25_IbF/LS25_VbR).

OGO-4 (SEQ ID NO:57) was constructed by replacing the pST3Gal1-CgtBS42 in OGO-5 with the CgtBS42-CST1 bicistronic gene amplified from CJL223 (primers LS26_IR/LS26_VF). The OGO-5 vector backbone, with the pST3Gal1-CgtBS42 portion omitted was amplified using primers LS26_VF/LS26_VR.

OGO-6 (SEQ ID NO:59) was constructed by replacing CST-I in OGO-4 with pST3Gal1 gene amplified from pMal-c5x-pST3Gal1-CgtBS42 (primers LS32_IF/LS32_IR). The OGO-4 (SEQ ID NO:57) vector backbone, minus CST-I was amplified using primers LS32_VF/LS32_VR.

OGO-7 (SEQ ID NO:60) was constructed by Gibson Assembly™ by inserting hST6GalNac4 downstream of CgtB* in OGO-5. hST6GalNAc4 with upstream RBS site was amplified from pETd-IFN+6A4 using LS38_2F and LS38_3Ra primers and the entire OGO-5 plasmid was amplified using LS38_3Fa/LS38_2R primers.

OGO-8 was constructed by Gibson Assembly™ by inserting hST6GalNac4 downstream of hppGalNAcT2 in OGO-5. hST6GalNAc4 with upstream RBS site was amplified from pETd-IFN+6A4 using LS44_IF and LS44_IR primers and the entire OGO-5 plasmid was amplified using LS44_VF/LS44_VR primers.

OGO-9 (SEQ ID NO:62) was constructed by Gibson Assembly™ by inserting hST6GalNac2 downstream of hppGalNAcT2 in OGO-5. hST6GalNAc2 with upstream RBS site was amplified from pETd-IFN+6A2 using LS45_IF and LS45b_IR primers and the entire OGO-5 plasmid was amplified using LS45b_VF/LS45_VR primers.

OGO-10 was constructed by Gibson Assembly™ by inserting the C. jejuni OH4382/84 Cst-II I53S variant with 32aa C-terminal deletion downstream of CgtB* in OGO-3. Cst-II was amplified from pET28a-CSTIIΔ32 (³⁶ using LS39_IF and LS39_IR primers and the entire OGO-3 plasmid was amplified using LS39_VF and LS39_VR primers.

OGO-11 was constructed from OGO-5 by deleting the CgtBS42 gene and inserting DmC1GalT (aa sequence) downstream of hppGalNAcT2. DmC1GalT with upstream RBS site was amplified from pET29a-DmC1GalT using LS47_1F/LS47_1R primers. The OGO-5 backbone was amplified in two parts (omitting the CgtBS42 gene) using LS47_VF/LS47_VR and LS47_2F/LS47_2R primers. OGO-11 was subsequently assembled via Gibson Assembly™.

TABLE 1
Primer Table

| | | SEQ | |
|---|---|---|---|
| | Primer | ID | Primer sequence |
| Construct | Name | NO | (5′ -> 3′) |
| pOSIP- | LS23-IF | 67 | GGGATCGGAATTCGA |
| neuABC | | | GCTCcacgacaggtt |
| | | | tcccgactg |
| | LS23-VR | 68 | cagtcgggaaacctg |
| | | | tcgtgGAGCTCGAAT |
| | | | TCCGATCCC |
| | LS23-VF | 69 | caacgtcgtgactgg |
| | | | gaaaacCATGGCGCC |
| | | | TAACCTAAACTGAC |
| | LS23-IR | 70 | GTCAGTTTAGGTTAG |
| | | | GCGCCATGgttttcc |
| | | | cagtcacgacgttg |
| pCW- | LS24_F | 71 | GAAGGAGATATACC |
| CgtBS42- | | | ATGACAAGGACTAGA |
| CSTI | | | ATG |
| | LS24_R | 72 | CATTCTAGTCCTTGT |
| | | | CATGGTATATCTCCT |
| | | | TC |
| pMAL- | LS19-VF | 73 | TATATTCAAATATAT |
| c5x- | | | AAAATAAAACCGTGA |
| pST3Gal1- | | | GAATTCCCTGCAGGT |
| CGTBS42 | | | AATTAAATAA |
| | | | GCTTC |
| | LS19-VR | 74 | gagtgcatgtgcatg |
| | | | ggcgCATATGTGAAA |
| | | | TCCTTCCCTCGATCC |
| | LS19-1F | 75 | GGATCGAGGGAAGGA |
| | | | TTTCACATATGcgcc |
| | | | catgcacatgcactc |
| | LS19-1R | 76 | CTCCTAAGCATCGAT |
| | | | GGATCttagcgacct |
| | | | ttaaaaatgcgaatc |
| | | | ttatt |
| | LS19-2F | 77 | aataagattcgcatt |
| | | | tttaaaggtcgctaa |
| | | | GATCCATCGATGCTT |
| | | | AGGAG |
| | LS19-2R | 78 | GAAGCTTATTTAATT |
| | | | ACCTGCAGGGAATTC |
| | | | TCACGGTTTTATTTT |
| | | | ATATATTTGA |
| | | | ATATA |
| OGO-5 | LS25_VF | 79 | TAAAACCGTGAGAAT |
| | | | TCCCTGatcgatgat |
| | | | aagctgtcaaacatg |
| | LS25_VaR | 80 | catccataatacctc |
| | | | ctgtcgactgtttcc |
| | | | gcatgcttattaac |
| | LS25_IaF | 81 | gttaataagcatgcg |
| | | | gaaacagtcgacagg |
| | | | aggtattatggatg |
| | LS25_IaR | 82 | GCTCATTTCAGAATA |
| | | | TTTGCCAttatttgc |
| | | | cgctggtcatttt |
| | LS25_IbF | 83 | aaaatgaccagcggc |
| | | | aaataaTGGCAAATA |
| | | | TTCTGAAATGAGC |
| | LS25_IR | 84 | catgtttgacagctt |
| | | | atcatcgatCAGGGA |
| | | | ATTCTCACGGTTTTA |
| | LS25_IbF | 85 | AATTGACCAACAAGG |
| | | | ACCATAGATTcatat |
| | | | gcgcccatgcacat |
| | LS25_VbR | 86 | atgtgcatgggcgca |
| | | | tatgAATCTATGGTC |
| | | | CTTGTTGGTCAATT |
| OGO-4 | LS26_IF | 87 | ATTGACCAACAAGGA |
| | | | CCATAGATTATGTTT |
| | | | AAAATTTCAATCATC |
| | | | TTACCA |
| | LS26_IR | 88 | gctcatgtttgacag |
| | | | cttatcaTTCTGCAG |
| | | | GTCGACTTATTTGTT |
| | LS26_VF | 89 | AACAAATAAGTCGAC |
| | | | CTGCAGAAtgataag |
| | | | ctgtcaaacatgagc |
| | LS26_VR | 90 | TGGTAAGATGATTGA |
| | | | AATTTTAAACATAAT |
| | | | CTATGGTCCTTGTTG |
| | | | GTCAAT |
| OGO-11 | LS47_VF | 91 | gattcgcatttttaa |
| | | | aggtcgctaa ATCG |
| | | | ATGATAAGCTGTCAA |
| | | | ACATGAG |
| | LS47_VR | 92 | CGCTCATATGTATAT |
| | | | CTCCTTCTTAAAGct |
| | | | agactactgctgcag |
| | | | gttgag |
| | LS47_1F | 93 | ctcaacctgcagcag |
| | | | tagtctag CTTTAA |
| | | | GAAGGAGATATACAT |
| | | | ATGAGCG |
| | LS47_1R | 94 | cgctaataagaattt |
| | | | tcataatacctcctt |
| | | | TTACTGGGTTTTGG |
| | | | TTTCTGCG |
| | LS47_2F | 95 | CGCAGAAACCAAAAC |
| | | | CCAGTAA aaggagg |
| | | | tattatgaaaattct |
| | | | tattagcg |
| | LS47_2R | 96 | CTCATGTTTGACAGC |
| | | | TTATCATCGAT tta |
| | | | gcgacctttaaaaat |
| | | | gcgaatc |
| OGO-8 | LS44-IF | 97 | ctcaacctgcagcag |
| | | | tagtctagGTATAAG |
| | | | AAGGAGATATACATA |
| | | | TGACCTGC |
| | LS44_VR | 98 | GCAGGTCATATGTAT |
| | | | ATCTCCTTCTTATAC |
| | | | ctagactactgctgc |
| | | | aggttgag |
| | LS44_VF | 99 | GTCGAGTCTGGTAAA |
| | | | GAAACCG aaggagg |
| | | | tattatgaaaattct |
| | | | tattagcg |
| | LS44_IR | 100 | cgctaataagaattt |
| | | | tcataatacctcctt |
| | | | CGGTTTCTTTACCAG |
| | | | ACTCGAC |
| OGO-9 | LS45_IF | 101 | ctcaacctgcagcag |
| | | | tagtctagGTATAAG |
| | | | AAGGAGATATACATA |
| | | | TGATGTCTAA |
| | | | GG |
| | LS45_VR | 102 | CCTTAGACATCATAT |
| | | | GTATATCTCCTTCTT |
| | | | ATACctagactactg |
| | | | ctgcaggttg |
| | | | ag |
| | LS45b_VF | 103 | GCTAAGTCGAGTCTG |
| | | | GTAAAGAAACaagga |
| | | | ggtattatgaaaatt |
| | | | cttattagcg |
| | LS45b_IR | 104 | cgctaataagaattt |
| | | | tcataatacctcctt |
| | | | GTTTCTTTACCAGAC |
| | | | TCGACTTAGC |
| OGO-10 | LS39_IF | 105 | ATAAAACCGTGAGAA |
| | | | TTCCCTGgtataaga |
| | | | aggagatataCATAT |
| | | | Gaaaaaag |
| | LS39_VR | 106 | cttttttCATATGta |
| | | | tatctccttcttata |
| | | | cCAGGGAATTCTCAC |
| | | | GGTTTTAT |
| | LS39_VF | 107 | caaaaaatattaatt |
| | | | tttaaGTCGAGtctg |
| | | | gatcgatgataagct |
| | | | gtcaaacatg |
| | LS39_IR | 108 | catgtttgacagctt |
| | | | atcatcgatccagaC |
| | | | TCGACttaaaaatta |
| | | | atattttttg |

### Engineered Origami 2™ (DE3) Strain for CMP-Neu5Ac Synthesis (OG2neu+)

A codon optimized neuABC gene cluster from Neisseria meningitidis (Geneart™), was ordered and cloned into a pACYC184 vector, modified with a triple tac promoter and a multiple cloning site from pCW-Ori+⁸, at the Ndel and SalI restriction sites. The neuABC gene cluster, including upstream triple tac promoter was inserted into the multiple cloning site of the pOSIP-KO vector¹⁷, by Gibson Assembly™. Gibson Assembly™ primers with homologous ends to the pOSIP-KO MCS were designed and used to amplify neuABC (along with its upstream triple tac promoter using primer pairs LS23-IF/IR). Likewise, primers with homologous ends to the pEPAC-3184 plasmids (upstream of the Tac promoter and downstream of the cloned gene) were designed to amplify the pOSIP-KO backbone (primer pairs LS23-VF/VR). Equimolar amounts of the PCR products (˜200-300 ng in total) were mixed with the Gibson Assembly™ mix (containing the T5 exozyme, Phusion™ polymerase and Taq ligase) and incubated at 50° C. for 1 hour. The reaction mix was then transformed directly into electrocompetent Origami2™ (DE3) E. coli cells (Novagen™, referred to as OG2), followed by outgrowth at 37° C. for 1 hour, before plating onto LB-Kan (15 μg/mL) plates. Successful integrants were confirmed by colony PCR using primers that anneal to the chromosomal DNA (at either side of the attB integration site) and to the pOSIP integration vector (186 primers described 17).

The pOSIP-KO integration cassette, encoding phage and Kan resistance cassette, was subsequently excised by the introduction of FLP recombinase encoded by pE-FLP vector¹⁷. The OG2 strain with inserted neuBCA gene is now referred to as OG2neu+.

### Expression and Purification of GB1-IFNα2b

OG2neu+strains harbouring GB1-IFNα2b and OGO-1/13/14/30/33 were grown in 50 mL 2xYT (100 μg/mL ampicillin, 30 μg/mL chloramphenicol), until mid-log phase, then induced with 0.5 mM IPTG and grown at 18° C. for 16 hr. Cells were lysed in 1.5 mL Bugbuster™ (in 20 mM Tris pH 8, 500 mM NaCl+40 mM imidazole) and purified by IMAC. Elution fractions were pooled and buffered exchanged into 50 mM ammonium bicarbonate pH 7.0 via 10 kDa MWCO Amicon™ concentrator.

### Activity Assay

hppGalNAcT activity—Pellet from 1 mL of overnight culture was lysed with 30 μL bugbuster™. 5 μL of cleared lysate was added to a reaction mixture containing 0.05 mM BODIPY-fetuin peptide (GAEAEAPSAVPDAAG), 1 mM UDP-GalNAc, 50 mM Hepes 7.5 and 10 mM MnCl₂, in a 10 μL assay reaction. The reaction mixture was incubated at 20° C. for 20 min before spotting on a silica gel TLC plate. The TLC was run using a 4:2:1:0.2 (EtOAc:MeOH:H₂O:HOAc) solvent system, and visualized on a UV tray.

Coupled CgtB+Gne activity—Lysate was prepared as above, and added to a reaction mixture containing 0.5 mM Coumarin-a-GalNAc (Tn), 1 mM UDP-Glc, 50 mM Hepes 7.5 and 10 mM MnCl₂, in a 10 μL assay reaction. Reaction was incubated at 20° C. for 20 min before spotting on a silica gel TLC plate. TLC was run using a 7:2:1 (EtOAc:MeOH:H2O) solvent system, and visualized on a UV tray.

Sialyltransferase activity—Lysate was prepared as above, and added to reaction mixture containing 0.5 mM BODIPY-α-TAg, 1 mM CMP-Sia, 50 mM Hepes 7.5 and 10 mM MnCl₂, in a 10 μL assay reaction. Reaction was incubated at 20° C. for 20 min before spotting on a silica gel TLC plate. TLC was run using a 4:2:1:0.2 (EtOAc:MeOH:H2O:HOAc) solvent system, and visualized on a UV tray.

CMP-NeuAc lysate detection—Lysate was prepared as above, and added to reaction mixture containing 0.1 mg/mL Cst-I, 0.5 mM BODIPY-Lac, 50 mM Hepes 7.5 and 10 mM MnCl₂, in a 10 μL assay reaction. Reaction was incubated at 20° C. for 40 min. TLC was run using a 4:2:1:0.2 (EtOAc:MeOH:H2O:HOAc) solvent system, and visualized on a UV tray.

### Intact-MASS Analysis Mass

Proteins were buffer-exchanged to 100 mM ammonium bicarbonate using Amicon™ ultrafiltration devices (SPECS!) and subsequently diluted to 3 ng/μL in 0.1% formic acid. Samples (5 μL per injection) were subjected to liquid chromatography with coupled electrospray mass spectrometry (Waters nanoACQUITY UPLC™ with Waters Xevo™ G2 qTOF mass spectrometer or Agilent™ 1200 HPLC system with Agilent™ 6550 qTOF mass spectrometer) equipped with a Zorbax™ 300SB-C8 column (Agilent™) and eluted using a gradient of 5% to 90% acetonitrile (0.1% formic acid). The protein elution peak was integrated and deconvolution from the multiple charged species was performed using MaxEnt1™ as part of Waters MassLynx 4.1™ when measured on the Xevo G2™ mass spectrometer or UniDec 3.1.0²⁵ followed by plotting with mMass²⁶ 5.5.0™ when measured on the Agilent™ system.

### Glycoform Analysis By HPLC

The HPLC method has been previously reported in Du et al.⁸.

### EXAMPLES

### Example 1: Engineered E. coli Strain for CMP-Neu5Ac Donor Synthesis

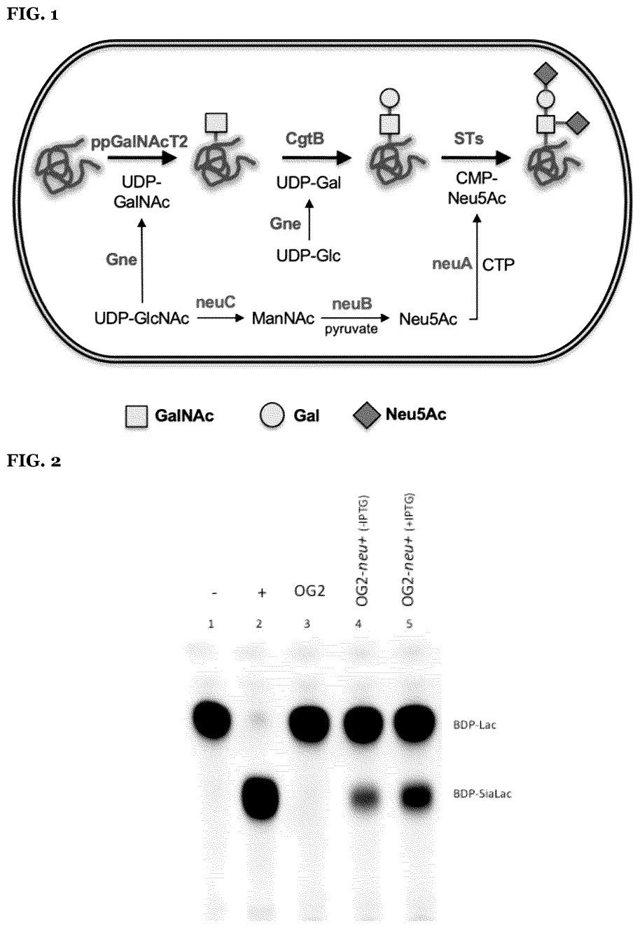

From our previous in vivo O-glycosylation studies, we identified the Origami 2™ (DE3) strain (OG2) as an ideal host for in vivo sialylation trials⁸. However, in order to provide intracellular donor substrate for sialyltransferases, we needed to incorporate a CMP-Sialic acid biosynthesis pathway into the strain. For this, we looked at the neuBCA operon from the bacterial strains that are able to make sialic acid de novo such E. coli K1, Neisseria meningitidis and C. jejuni. The neuBCA operon encodes three enzymes that convert the existing intracellular pool of UDP-GlcNAc (normally used for cell wall biosynthesis) into CMP-NeuAc. NeuC is a hydrolysing UDP-GlcNAc 2′ epimerase which generates free ManNAc¹⁵, neuB is the sialic acid synthetase, condensing ManNAc with pyruvic acid, and neuA is the CMP-NeuAc synthetase, coupling sialic acid to CMP¹⁶. (FIG. 1).

The N. meningitidis neuBCA operon (2.9 kB) was inserted into the chromosome using the clonetegration technology described by St-Pierre et al.¹⁷ to form the OG2neu+ strain¹⁷. Several positive integrant clones were grown and checked for functional neuBCA operon by assaying cell lysates for the presence of CMP-Neu5Ac. This was detected via the conversion of the fluorescent acceptor substrate BODIPY-Lac to BODIPY-SiaLac using C. jejuni α2,3-sialyltransferase Cst-I (FIG. 2). Indeed the presence of CMP-Neu5Ac was detected only in the engineered OG2neu+ cells and not in native OG2 cells, with a smaller amount in the non-induced OG2-neu+ strain, likely due to some leaky expression.

### Example 2: Construction of Sialylation Operons

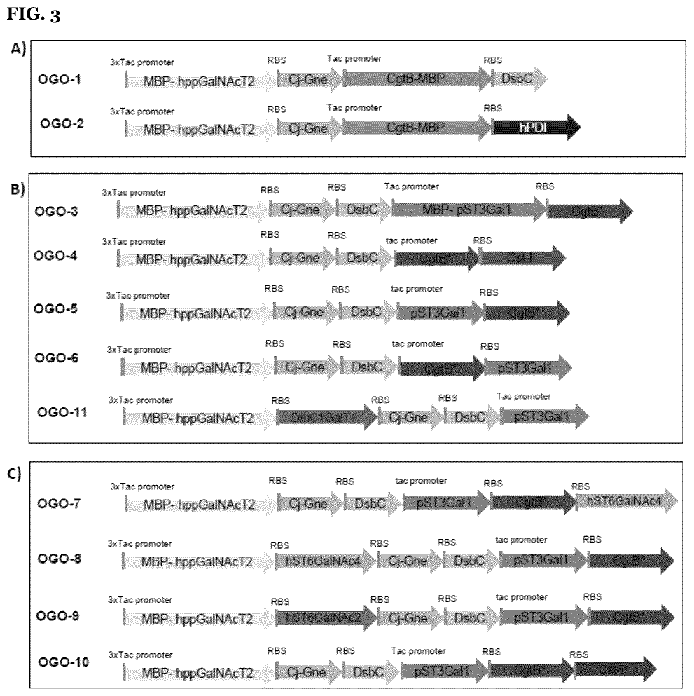

The sialylation operons constructed used OGO-1⁸ as a point of reference (FIG. 3a). A few modifications were made including repositioning the DsbC gene behind Gne, forming a GalNAcT2-Gne-DsbC tri-cistronic operon under control of a triple tac promoter. Secondly, the MBP-fused CgtB gene was replaced by an enhanced tagless variant (CgtB*) identified from directed evolution¹⁸. Based on our previous success with use of recombinant α-2,3-sialyltransferases C. jejuni Cst-I and the porcine ST3Gal1 for synthesizing Sia-TAg in vitro, we selected these as our candidates to incorporate into the operon. Under a second tac promoter, the bi-cistronic operons MBP-pST3GalI-CgtB* or CST-I-CgtB* were inserted to create OGO-3 (SEQ ID NO:56) and OGO-4 (SEQ ID NO:57), respectively (FIG. 3b). The upstream MBP gene from pST3Gal1 from OGO-3 was subsequently removed to form OGO-5.

The disialylation operons were constructed using OGO-5 as a starting point. We opted to test two human GT29 enzymes capable of core GalNAcα-2,6-sialylation, hST6GalNAc2 and hST6GalNAc4^{19,20}. Preliminary work has shown that both enzymes can be recombinantly expressed in E. coli and are active in vitro on the protein acceptor fetuin. Two versions containing hST6GalNAc4 were constructed, one with the gene inserted downstream of CgtB* (OGO-7 (SEQ ID NO:60)) and one inserted downstream of MBP-hppGalNacT2 (OGO-8 (SEQ ID NO:61)). The hST6GalNAc2 containing operon, OGO-9 (SEQ ID NO:62), was constructed in a similar fashion to OGO-8 except with hST6GalNAc2 in place of hST6GalNAc4 (FIG. 3c).

### Example 3: Production of Sia-Tag-modified GB1-IFNα2b

To verify the activities of the newly incorporated sialyltransferases in OGO-3 (SEQ ID NO:56) and OGO-4 (SEQ ID NO:57), assays were performed with cell lysates. Indeed, both operons produced enzymes that sialylated the small molecule BODIPY-TAg substrate, suggesting that the expressed MBP-pST3Gal1 and CST-I were functional in OGO-3 and OGO-4, respectively. However, when the other activities in the OGO-3 (SEQ ID NO:56) operon were assayed, it was discovered that, compared to our control OGO-1 operon, the hppGalNAcT2 activity in OGO-3 was severely decreased such that we were not able to detect any GB1-IFnα2b sialylation in our OGO-3 co-expression trials. We postulated that this decrease in expression was a result of plasmid instability and possible recombination as a consequence of having two MBP genes of similar sequence in the same construct. Since we determined that pST3Ga1 does not require the MBP tag for activity, it was removed in the subsequent OGO-5 construct.

After removal of the MBP gene, hppGalNAcT2 expression levels and activity in the OGO-5 lysates were restored and displayed even higher activity compared to OGO-1 and OGO-4. Likewise the CgtB* activity of OGO-4 and OGO-5 was higher than that of OGO1, most likely because they contained the enhanced CgtB* variant. Finally the sialyltransferase activities of OGO-4 and OGO-5 were comparable in vitro.

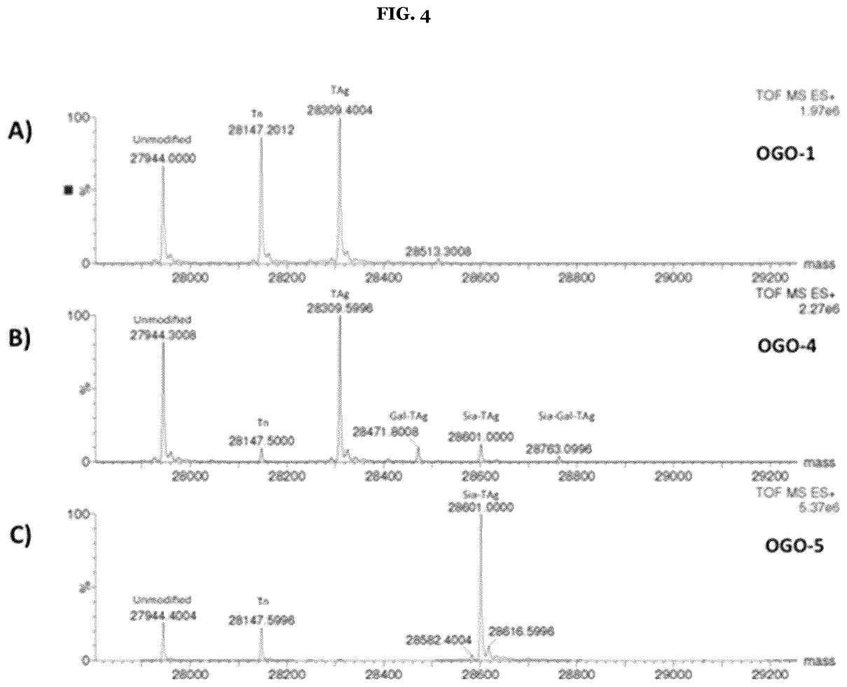

After confirming that both operons expressed active enzymes, the abilities of the operons to modify proteins in vivo were tested. To do this our target GB1-IFNα2b was co-expressed with either OGO1, OGO-4 or OGO-5 in the OG2-neu+strain, then purified and analyzed by intact mass spectrometry to identify the glycoforms (FIG. 4). Sia-TAg modified GB1-IFNα2b could be detected in both OGO-4 and OGO-5 co-expressed strains (28601.0 Da). However, overall levels of sialylation are higher with OGO-5. In addition, no T-Ag was detected, signifying complete conversion of T-Ag to Sia-TAg. HPLC quantitation of GB1-IFNα2b glycoforms further confirmed that the sample is predominantly modified with Sia-TAg (˜85%), along with some minor peaks for unmodified (˜12%) and Tn modified (˜3%) protein, but sign of TAg modification.

In comparison, OGO-4 (SEQ ID NO:57) co-expression resulted in a more heterogenous glycoform distribution with lower overall sialylation. The majority of the GB1-IFNα2b was either unmodified (27944.3 Da) or T-Ag modified (28309.6 Da), along with small amounts of Tn (28471.5 Da), di-galactosylated Tn (28471.8 Da), and sialylated, di-galactosylated Tn (28763.1 Da). Interestingly, we noticed that the target GB1-IFNα2b expression levels in OG2-neu+were compromised when OGO-4 was co-expressed. In this case, overall yields of GB1-IFNα2b were approximately 2.5-3× lower than in our OGO1or OGO-5 co-expression strains.

We also switched the order of pST3Gal1 and CgtB* in OGO-5, to make OGO-6 (FIG. 3B). However, co-expression trials and intact mass analysis indicated that the two operons performed similarly in modifying GB1-IFNα2b.

### Example 4: Production of diSia-TAg Modified GB1-IFNα2b Using ST6GalNAc4

Given our initial success at production of Sia-TA-modified GB1-IFNα2b with OGO-5, we set off to make diSia-TAg by incorporation of hST6GalNAc4, which works uniquely on SiaTAg²¹. This disialylation operon variant containing hST6GalNAc4, OGO-7 (FIG. 3C), was thus constructed and co-expressed with GB1-IFNα2b. Intact mass analysis of the target protein product showed that the majority of the modification is Sia-TAg, with a smaller amount of diSia-TAg (28893.3 Da), Tn, and unmodified form. As with OGO-5, no TAg was detected. This version of the disialylation operon therefore appears to be functional, but the conversion of Sia-TAg to diSia-TAg needed to be optimized to obtain homogeneous glycosylation. One way to accomplish this was to increase the hST6GalNAc4 transcript level, and thus activity, by shifting its position upstream in the operon where it would be under the influence of a stronger promoter. OGO-8 (FIG. 3C) was thus constructed and lysate activities of the resulting OGO-8 (SEQ ID NO:61) expression trials indeed showed considerably higher in vitro hST6GalNAc4 activity compared to that of OGO-7 (SEQ ID NO:60).

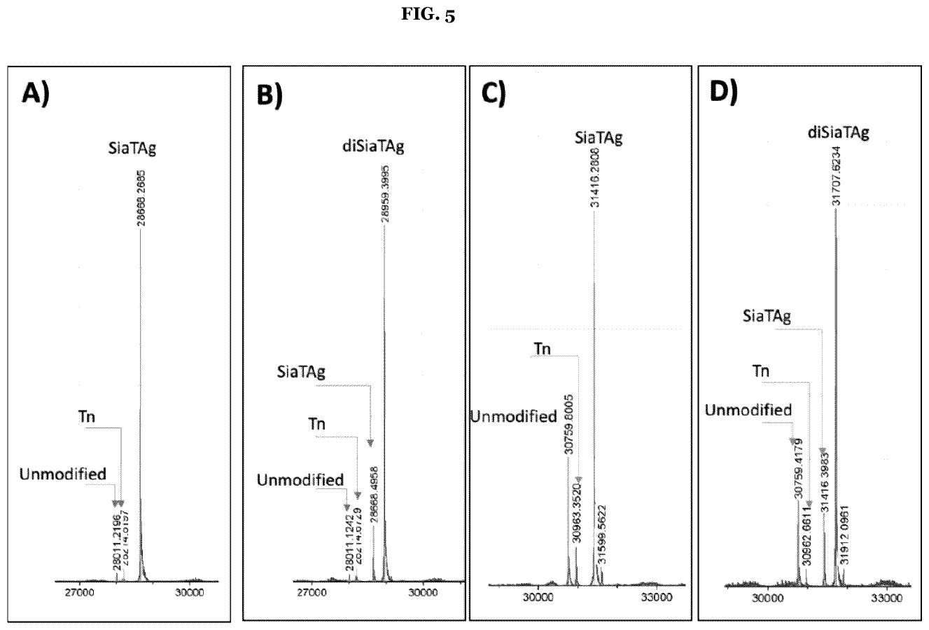

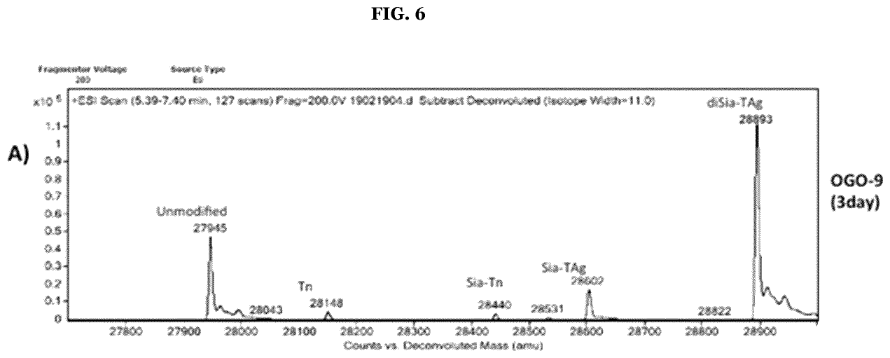

Consistent with this, in vivo co-expression trials with GB1-IFNα2b revealed that OGO-8 (SEQ ID NO:61) outperforms OGO-5 (SEQ ID NO:58) and produces a glycosylated protein with diSia-TAg as the major glycoform, along with some unmodified, Tn and only traces of Sia-TAg (FIG. 5C).

### Example 5: Sialylation of Other Target Proteins Using OGO-5 & OGO-8 Operons

Importantly OGO-5 (SEQ ID NO:58) and OGO-8 (SEQ ID NO:61) are also effective at glycosylating other target proteins. This includes a glycosylation sequon-optimized version of GB1-IFNα2b wherein the GVGVT¹⁰⁶ region is replaced by GPQPT¹⁰⁶ (referred to as GB1-IFNα2b*), which we have previously shown to be a better substrate for hppGalNAcT2⁸ (FIGS. 5A and B). We also showed that the sequon-optimized version of recombinant human growth hormone (GB1-hGH*) could be modified by both sialylation operons⁸ (FIG. 5C and D). The predominant glycoforms observed were SiaTAg from OGO-5 (SEQ ID NO:58) co-expression, and diSia-TAg from OGO-8 (SEQ ID NO:61) co-expression as anticipated.

### Example 6: Production of Sialylated Core GaINAc-modified GB1-IFNα2b Using ST6GalNAc2

Even though hST6GalNAc4 was performing well we investigated the in vivo activity of another core GalNAc sialyltransferases with broader specificity, hST6GalNAc2. By contrast with hST6GalNAc4, which acts only on SiaTAg, hST6GalNAc2 has been shown to sialylate, in decreasing order of preference, TAg, SiaTAg and Tn²². The resulting hST6GalNAc2 disialylation operon , OGO-9 (FIG. 3C) was shown to primarily produce diSia-TAg modified GB1-IFNα2b, along with some unmodified, Tn, Sia-Tn and Sia-TAg (FIG. 6). This is similar to what was seen with OGO-8 (SEQ ID NO:61), though with higher Sia-TAg levels that are likely made up from a mixture of Siaα2,3-TAg and Ga1β1,3(Siaα2,6)GalNAc glycoforms.

We also investigated the potential for hST6GalNAc2 to synthesise different mono-sialylated structures in vivo. To this end a pETduet expression vector (pETd-IFN+6A2) co-expressing GB1-IFNα2b and hST6GalNAc2, in the upstream and downstream cloning sites respectively, was constructed. This plasmid was co-expressed with OGO1 and the intact mass of the interferon product was determined, revealing that both Sia-Tn and core GalNAc Sia-TAg glycoforms could be made in vivo.

An E. coli strain, OG2-neu+, that can synthesize CMP-Neu5Ac de novo and the construction of mono and di-sialylation operons incorporating various bacterial or mammalian sialyltransferases is described. It was fortuitously discovered that the mammalian sialyltransferases pST3Gal1, hST6GalNAc2 and hST6GalNAC4 could be used to sialylate protein targets in vivo in the E. coli host cytoplasm.

From the mono-sialylation operons it was possible to achieve ˜85% Sia-TAg modification with the OGO-5 operon (SEQ ID NO:58). It is interesting that although cell extract from OGO-4 (containing CST-I) and OGO-5 (containing pST3Gal1) expression both showed comparable operon activities in vitro, it is not mirrored in their performances in vivo. A possible explanation of the poorer performance by OGO-4 (SEQ ID NO:57) is that as the bacterial CST-I usually works on lipopolysaccharide synthesis, and there is reduced protein targeting efficiency. In comparison, the in vivo performance of pST3Gal1 is robust and is efficient at converting all TAg to Sia-TAg.

Building upon the OGO-5 (SEQ ID NO:58) mono-sialylation operon, the disialylation operons OGO-8 (SEQ ID NO:61) and OGO-9 (SEQ ID NO:62) were shown to successfully further modify target proteins with diSia-TAg, via the introduction of hST6GalNAc4 and hST6GalNAc2, respectively. In another test case with hST6GalNAc2, we also demonstrated the ability to make a variety mono-sialylated structures, including Sia-TAg and Sia-Tn. Taken together, this will give thus us the flexibility to design specific operons for the selected structures desired.

Gene placement within the operon appeared to matter for certain of the mammalian glycosyltransferases. For example, the pST3Gal1 activity appears to be robust, as could be placed behind a single tac promoter as in OGO-5 (SEQ ID NO:58) and OGO-6 (SEQ ID NO:59). However, hST6GalNAc4 was required to be placed behind the stronger triple tac promoter, as in OGO-8 (SEQ ID NO:61), in order to be as efficient at converting Sia-TAg to diSia-TAg.

It appears that formation of the Tn antigen is the primary limiting factor in general for the operons. At least with OGO-5 (SEQ ID NO:58) and OGO-7 (SEQ ID NO:60) operons, once the GalNAc is attached by hppGalNAcT2, the rest of the enzymes in the operons are able to build the rest of the SiaTAg/diSia-TAg structure with no major roadblocks. However, we have shown that we can improve the initial Tn antigen formation by using an engineered O-glycosylation site that is optimal for ppGalNAcT2.

The current application shows that the production of sialylated mucin-like O-glycans is achievable in a bacterial expression system and we expect in the future to further expand on the target proteins repertoire (in addition to IFNα2b and hGH) that can be successfully used in this system.

### Example 7: Variation of Core-1 Synthase Mono-sialylation Operon

The mono-sialylation operon Drosophila melanogaster C1GalT1 (DmC1GalT1) galactosyltransferase was tested as a replacement for CgtB*²⁷. Unlike its mammalian counterpart, which requires the chaperone Cosmc to function, DmC1GalT1 does not require assistance. In previous studies by others the DmC1GalT1 β-1,3-galactosyltransferase had been used successfully in the synthesis of T-Ag on Muc1 peptide in yeast²⁸. However, while it has been expressed recombinantly in E. coli for the in vitro modification of Tn modified glycoproteins, it has yet to be proven to modify glycoproteins within the E. coli cytoplasm.

In a preliminary construct, we replaced the CgtB* gene in OGO-5 (SEQ ID NO:58) with that of DmC1GalT1. Although we could detect trace activity of DmC1GalT1 in cell lysate, no T-Ag could be detected on GB1-IFNα2b in vivo (data not shown). We then constructed a new OGO (not shown), where the DmC1GalT1 gene was shifted to a position upstream in the operon where it would be under the influence of a stronger promoter. In this instance, when this new OGO was co-expressed, small amounts of Sia-TAg could be detected on GB1-IFNα2b, however most of the target was only Tn modified (data not shown). This bottleneck in synthesizing T-Ag, and subsequently Sia-TAg, suggests that within the E. coli cytoplasm, DmC1GalT1 may not be as effective as CgtB*.

Although in vitro studies show that lysates from E. coli expressing CgtB* or DmC1GalT1 are comparably active against both small molecule Tn substrates, DmC1GalT1 requires Mn²⁺ for activity²⁹. Since levels of Mn²⁺ are tightly regulated in E. coli^{30,31}, this may explain the limited activity of DmC1GalT1 in our in vivo system. Conversely, CgtB* can utilize either Mn²⁺ or Mg²⁺ ion, the latter being present at concentrations 100-1000 fold higher compared to Mn²⁺³⁰. The Mn²⁺ concentration has been also shown to be important for the in vivo synthesis of core-3 structures in S. cerevisiae, due to the stringent metal requirement of the β3GnT6 Core 3 synthase³². Nevertheless, DmC1GalT1 is a possible candidate in cells where Mn²⁺ is abundant.

### Example 8: Kdo Transferase Activity of pST3Gal1

During our expression trials with OGO-3 (SEQ ID NO:56), we observed an unexpected modification to GB1-IFNα2b* when it was co-expressed in the parental Origami 2™ (DE3) strain that does not synthesize CMP-Neu5Ac. In addition to the T-Ag modification we had expected at 28377.7 Da, there was a predominant peak corresponding to a mass of 28597.9 Da (data not shown). The +220 Da mass difference corresponds to a Kdo (2-keto-3-deoxyoctonate) saccharide. Kdo is a component of lipopolysaccharide and thus it would be expected the donor CMP-Kdo substrate would be found in the cytoplasm. Further studies revealed that pST3Gal1 can transfer Kdo to BODIPY-TAg in vitro (data not shown). As we have never observed this Kdo modification in the OG2(DE3)neu+ strain, this suggests that pST3Gal1 preferentially uses CMP-Neu5Ac over CMP-Kdo and only carries out Kdo transfer when CMP-Neu5Ac is absent.

Such Kdo transfer has been reported in one previous account in which Kdo-α-2,6-lactose was synthesized in metabolically engineered E. coli³³. In that case theα-2,6-sialyltransferase from JT-ISH-224 preferentially utilized CMP-Kdo over the CMP-Neu5Ac produced by a plasmid expressing the C. jejuni neuABC genes, at the concentrations in which they were present. We believe our report represents the first example of a mammalian ST exhibiting Kdo transferase activity.

### Example 9: Oligosialylation Operon Using Cst-II

To see if oligosialylated structures might be created by extending the Sia-TAg structure created by OGO-5 (SEQ ID NO:58) using the bifunctional α-2,3/2,8 sialyltransferase Cst-II from C. jejuni³⁴, whereby this might allow a possible further modification by a bacterial polysialyltransferase, which requires a minimum disialylated primer as an acceptor³⁵. Cst-II was previously used as a priming enzyme allowing the polysialylation of a target protein within the E. coli cytoplasm⁷.

Based on our previous observations that Cst-II could act on Sia-TAg fluorescent acceptor in vitro, as well as on protein modified with Sia-TAg (data not shown), a new OGO operon OGO-10 was constructed by adding Cst-II^{34, 36} downstream of CgtB* in OGO-5 (see FIG. 3).

Co-expression of our IFN gene with the new OGO operon with added Cst-II revealed that the GB1-IFNα2b so produced is modified with a mix of glycoforms including those corresponding to: unmodified, Tn, TAg, Sia-TAg, Sial-TAg. This suggests that although disialylation is possible with new OGO operon with added Cst-II, the overall product profile is quite heterogeneous, and a significant portion of the target protein remains unmodified. Additionally, a mass at 28530.7 Da suggests a Kdo-TAg modification, as previously observed in the expression of OGO-5 (SEQ ID NO:58) in Origami 2™ (DE3) (data not shown).

This disproportionately low level of sialylation may be related to the fact that, in vitro, Cst-II can hydrolyze CMP-Neu5Ac in the absence of an acceptor.³⁶ It is thus possible that the cytoplasmic levels of CMP-Neu5Ac are depleted in the new OGO operon with added Cst-II co-expression strain, resulting in the utilization of CMP-Kdo by pST3Gal1. Further tweaking of the system will thus be required to optimize the Cst-II activity, possibly by following the approach of Drouillard et al. to eliminate the KDO addition by increasing the CMP-Neu5Ac levels33.

### Example 10: Co-expression of QSox1b Enables OGO-8 Activity in JM109(DE3) Strain

O-glycoproteins are usually produced in strains that have trxB and gor mutations, including OG2(DE3) and ShuffleT7Express⁸. Nguyen et al. showed that a combination of a sulfhydryl oxidase and a protein disulfide isomerase can work to produce disulfide-containing proteins in strains with intact reducing pathways37. Since our OGO-8 (SEQ ID NO:61) construct already contains a protein disulfide isomerase (dsbC), only the sulhydryl oxidase would need to be introduced. Accordingly, we found that active ppGalNAcT2, pST3Gal1, and hST6GalNAc4 could be produced in strains with intact reducing pathways, if co-expressed with a hPDI-quiescin-sulfydryl oxidase fusion protein (hPDI-QSOX1b)³⁸ (data not shown).

Next, we tested whether we could produce sialylated GB1-IFNα2b* in a JM109(DE3) strain. Similar to the construction of the Origami 2™ (OG2) (DE3)neu+, the N. meningitidis neuCAB operon was inserted into the genome of JM109(DE3) using the integrase approach to make the JM109(DE3)-neu+ strain. Then, a pETduet vector was constructed with GB1-IFNα2b* in the MCS1 site and QSOX1b in the MCS2 site. These vectors were subsequently co-expressed with the disialylation operon OGO-8 (SEQ ID NO:61) in the JM109(DE3)-neu+ strain.

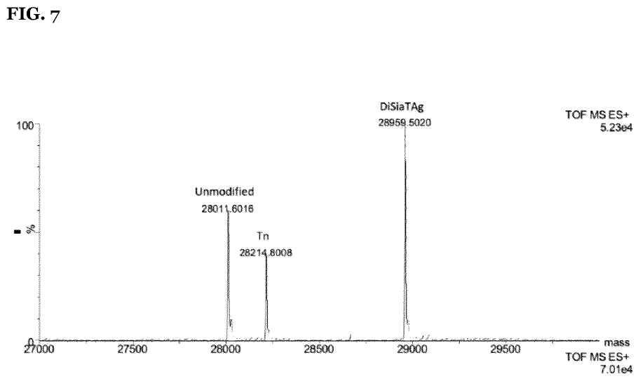

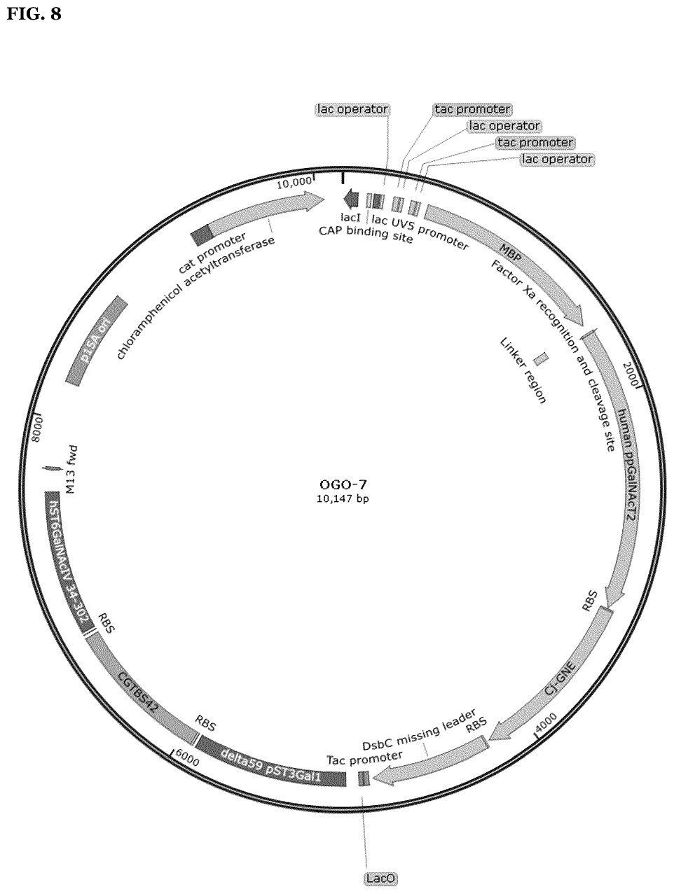

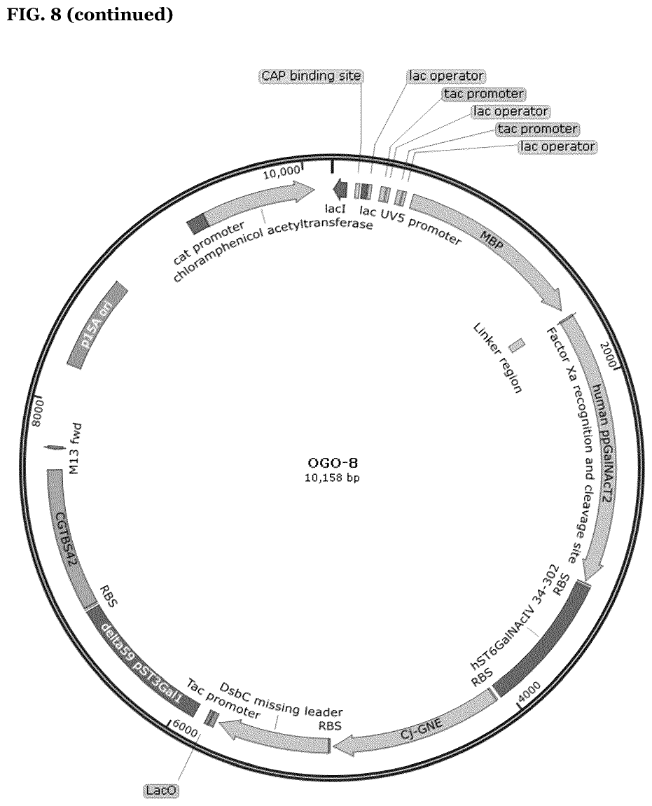

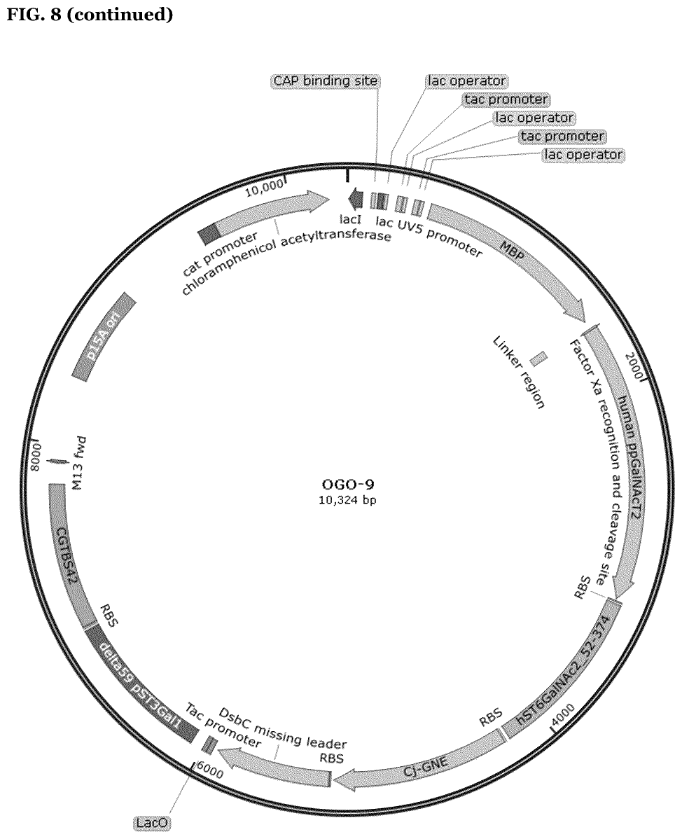

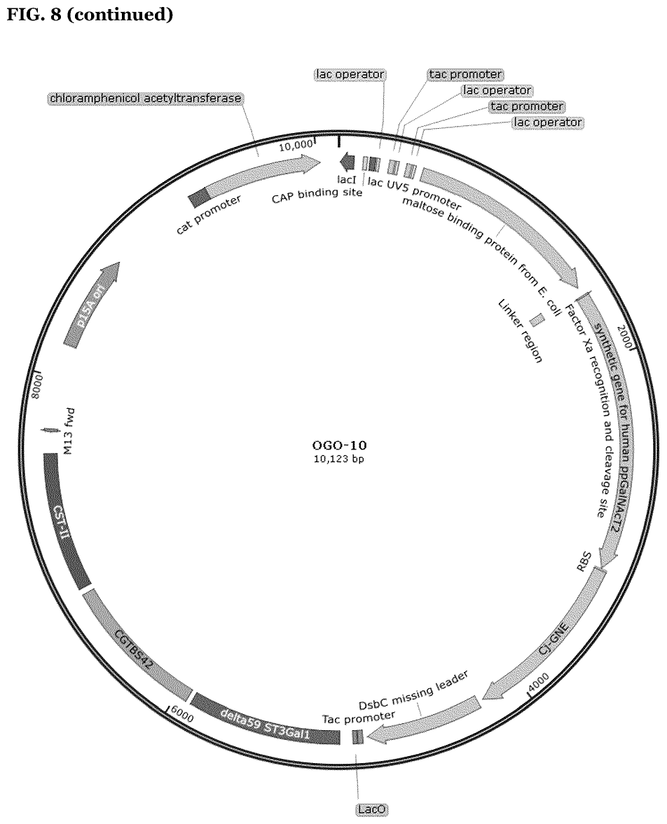

Gratifyingly, co-expression with OGO-8 (SEQ ID NO:61) and QS0x1b in JM109(DE3)-neu+ produced di-SiaTAg modified GB1-IFNα2b*, along with some Tn and unmodified protein (FIG. 7). The lack of T-Ag and Sia-TAg intermediates suggests that the pST3Gal1 and hST6GalNAc4 activities are sufficient, and that the bottleneck lies in the ppGalNAcT2 activity.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

1. Dicker, M. & Strasser, R. Using glyco-engineering to produce therapeutic proteins. Expert Opinion on Biological Therapy 15, 1501-1516 (2015).

2. Wells, E. & Robinson, A. S. Cellular engineering for therapeutic protein production: product quality, host modification, and process improvement Biotechnol. J. 12, 1600105-14 (2016).

3. Lombard, V., Golaconda Ramulu, H., Drula, E., Coutinho, P. M. & Henrissat, B. The carbohydrate-active enzymes database (CAZy) in 2013. Nucleic Acids Research 42, D490-5 (2014).

4. Wacker, M. et al. N-linked glycosylation in Campylobacter jejuni and its functional transfer into E. coli. Science 298, 1790-1793 (2002).

5. Keys, T. G. & Aebi, M. Engineering protein glycosylation in prokaryotes. Current Opinion in Systems Biology 5, 23-31 (2017).

6. Cuccui, J. et al. The N-linking glycosylation system from Actinobacillus pleuropneumoniae is required for adhesion and has potential use in glycoengineering. Open Biol 7, 160212 (2017).

7. Keys, T. G. et al. A biosynthetic route for polysialylating proteins in Escherichia coli. Metabolic Engineering 44, 293-301 (2017).

8. Du, T. et al. A Bacterial Expression Platform for Production of Therapeutic Proteins Containing Human-like O-Linked Glycans. Cell Chemical Biology 26, 203-212.e5 (2019).

9. Morell, A. G., Gregoriadis, G., Scheinberg, I. H., Hickman, J. & Ashwell, G. The role of sialic acid in determining the survival of glycoproteins in the circulation. J. Biol. Chem. 246, 1461-1467 (1971).

10. Solá, R. J. & Griebenow, K. Glycosylation of therapeutic proteins: an effective strategy to optimize efficacy. BioDrugs 24, 9-21 (2010).

11. Castilho, A. et al. Engineering of Sialylated Mucin-type O-Glycosylation in Plants. J. Biol. Chem. 287, 36518-36526 (2012).

12. Lobstein, J. et al. SHuffle, a novel Escherichia coli protein expression strain capable of correctly folding disulfide bonded proteins in its cytoplasm. Microbial Cell Factories 11, 56 (2012).

13. Rao, F. V. et al. Structural insight into mammalian sialyltransferases. Nat. Struct. Mol. Biol. 16, 1186-1188 (2009).

14. Ortiz-Soto, M. E. & Seibel, J. Expression of Functional Human Sialyltransferases ST3Gal1 and ST6Gal1 in Escherichia coli. PLoS ONE 11, e0155410 (2016).

15. Vann, W. F. et al. The NeuC protein of Escherichia coli K1 is a UDP N-acetylglucosamine 2-epimerase. Journal of Bacteriology 186, 706-712 (2004).

16. Vimr, E. R., Kalivoda, K. A., Deszo, E. L. & Steenbergen, S. M. Diversity of microbial sialic acid metabolism. MicrobioL Mol. Biol. Rev. 68, 132-153 (2004).

17. St-Pierre, F. et al. One-Step Cloning and Chromosomal Integration of DNA. ACS Synth. Biol. 2, 537-541 (2013).

18. Yang, G. et al. Fluorescence activated cell sorting as a general ultra-high-throughput screening method for directed evolution of glycosyltransferases. J. Am. Chem. Soc. 132, 10570-10577 (2010).

19. Harduin-Lepers, A. et al. Cloning, expression and gene organization of a human Neu5Ac alpha 2-3Gal beta 1-3GalNAc alpha 2,6-sialyltransferase: hST6GalNAcIV. Biochem. J. 352 Pt 1, 37-48 (2000).

20. Samyn-Petit, B., Krzewinski-Recchi, M. A., Steelant, W. F., Delannoy, P. & Harduin-Lepers, A. Molecular cloning and functional expression of human ST6GalNAc II. Molecular expression in various human cultured cells. Biochim. Biophys. Acta 1474, 201-211 (2000).

21. Harduin-Lepers, A. et al. Cloning, expression and gene organization of a human Neu5Ac alpha 2-3Gal beta 1-3GalNAc alpha 2,6-sialyltransferase: hST6GalNAcIV. Biochem. J. 352 Pt 1, 37-48 (2000).

22. Kono, M. et al. Redefined Substrate Specificity of ST6GalNAc II: A Second Candidate Sialyl-Tn Synthase. Biochemical and Biophysical Research Communications 272, 94-97 (2000).

23. Wakarchuk, W. W. et al. Thermostabilization of the Bacillus circulans xylanase by the introduction of disulfide bonds. Protein Eng. 7, 1379-1386 (1994).

24. Chiu, C. P. C. et al. Structural analysis of the alpha-2,3-sialyltransferase Cst-I from Campylobacter jejuni in apo and substrate-analogue bound forms. Biochemistry 46, 7196-7204 (2007).

25. Marty, M. T. et al. Bayesian deconvolution of mass and ion mobility spectra: from binary interactions to polydisperse ensembles. Anal. Chem. 87, 4370-4376 (2015).

26. Niedermeyer, T. H. J. & Strohalm, M. mMass as a software tool for the annotation of cyclic peptide tandem mass spectra. PLoS ONE 7, e44913 (2012).

27. Muller, R. et al. Characterization of mucin-type core-1 betal-3 galactosyltransferase homologous enzymes in Drosophila melanogaster. FEBS Journal 272, 4295-4305 (2005).

28. Amano, K. et al. Engineering of mucin-type human glycoproteins in yeast cells. Proc. Natl. Acad. Sci. U.S.A. 105, 3232-3237 (2008).

29. Gao, Y. et al. Acceptor specificities and selective inhibition of recombinant human Gal- and GlcNAc-transferases that synthesize core structures 1,2,3 and 4 of O-glycans. BBA—General Subjects 1830, 4274-4281 (2013).

30. Finney, L. A. & O'Halloran, T. V. Transition metal speciation in the cell: insights from the chemistry of metal ion receptors. Science 300, 931-936 (2003).

31. Martin, J. E., Waters, L. S., Storz, G. & Imlay, J. A. The Escherichia coli Small Protein MntS and Exporter MntP Optimize the Intracellular Concentration of Manganese. PLoS Genet 11, e1004977-31 (2015).

32. Saito, F., Sakamoto, I., Kanatani, A. & Chiba, Y. Manganese ion concentration affects production of human core 3 O-glycan in Saccharomyces cerevisiae. BBA—General Subjects 1860, 1809-1820 (2016).

33. Drouillard, S., Mine, T., Kajiwara, H., Yamamoto, T. & Samain, E. Efficient synthesis of 6′-sialyllactose, 6′ -disialyllactose, and 6′ -KDO-lactose by metabolically engineered E. coli expressing a multifunctional sialyltransferase from the Photobacterium sp. JT-ISH-224. Carbohydrate Research 345, 1394-1399 (2010).

34. Gilbert, M. et al. The Genetic Bases for the Variation in the Lipo-oligosaccharide of the Mucosal Pathogen, Campylobacter jejuni. J. Biol. Chem. 277, 327-337 (2001).

35. Willis, L. M., Gilbert, M., Karwaski, M. F., Blanchard, M. C. & Wakarchuk, W. W. Characterization of the -2,8-polysialyltransferase from Neisseria meningitidis with synthetic acceptors, and the development of a self-priming polysialyltransferase fusion enzyme. Glycobiology 18, 177-186 (2007).

36. Chiu, C. P. C. et al. Structural analysis of the sialyltransferase CstII from Campylobacter jejuni in complex with a substrate analog. Nat. Struct. Mol. Biol. 11, 163-170 (2004).

37. Van Dat Nguyen et al. Pre-expression of a sulfhydryl oxidase significantly increases the yields of eukaryotic disulfide bond containing proteins expressed in the cytoplasm of E. coli. Microb Cell Fact. 1-13 (2011). doi:10.1186/1475-2859-10-1.

38. Zhang et al. Highly efficient folding of multi-disulfide proteins in superoxidizing Escherichia coli cytoplasm. Biotechnol Bioeng. 1-8 (2014). doi:10.1002/bit.25309/abstract).

1. A plasmid, the plasmid comprising DNA encoding:
(a) a polypeptide N-acetylgalactosaminyltransferase;
(b) a β-1,3-galactosyltransferase;
(c) an UDP-Glc/GlcNAc 4-epimerase;
(d) a disulfide bond isomerase;
(e) an α-2,3-sialyltransferase; and
(f) an α-2,6-sialyltransferase.

2. The plasmid of claim 1, wherein the plasmid comprises at least 2 operons, wherein the DNA encoded in operon 1 comprises:
(i) at least 1 promoter;
(ii) a polypeptide N-acetylgalactosaminyltransferase;
(iii) a disulfide bond isomerase; and
(iv) an UDP-Glc/GlcNAc 4-epimerase;
and wherein the DNA encoded in operon 2 comprises:
(v) at least 1 promoter;
(vi) a β-1,3-galactosyltransferase;
(vii) an α-2,3-sialyltransferase; and
(viii) an α-2,6-sialyltransferase.

3. The plasmid of claim 1, wherein the plasmid comprises at least 2 operons, wherein the DNA encoded in operon 1 comprises:
(i) at least 1 promoter;
(ii) a polypeptide N-acetylgalactosaminyltransferase;
(iii) a disulfide bond isomerase;
(iv) an UDP-Glc/GlcNAc 4-epimerase; and
(v) an α-2,6-sialyltransferase; and wherein the DNA encoded in operon 2 comprises:
(vi) at least 1 promoter;
(vii) a β-1,3-galactosyltransferase; and
(viii) an α-2,3-sialyltransferase.

4. The plasmid of claim 1, 2 or 3, further comprising a ribosomal binding site encoded upstream of the start codon of each encoded gene.

5. The plasmid of claim 2, 3 or 4, wherein the promoter in operon 1 and operon 2 are selected from: an inducible promoter and a constitutive promoter.

6. The plasmid of any one of claims 2-5, wherein there are three copies of the promoter in operon 1 and one copy of the promoter in operon 2.

7. The plasmid of any one of claims 1-6, wherein the polypeptide N-acetylgalactosaminyltransferase is human polypeptide N-acetylgalactosaminyltransferase 2 (hppGalNAcT2).

8. The plasmid of any one of claims 1-7, wherein the β-1,3-galactosyltransferase is selected from: Campylobacter jejuni β-1,3-galactosyltransferase (CgtB); and Drosophila melanogaster C1GalT1 galactosyltransferase (DmC1GalT1).

9. The plasmid of any one of claims 1-8, wherein the UDP-Glc/GlcNAc 4-epimerase is Campylobacter jejuni UDP-Glc/GlcNAc 4-epimerase (Cj-Gne).

10. The plasmid of any one of claims 1-9, wherein the disulfide bond isomerase is selected from: human disulfide bond isomerase (hPDI); and E. coli disulfide bond isomerase (DsbC). ii. The plasmid of any one of claims 1-10, wherein the α-2,3-sialyltransferase is selected from: Campylobacter jejuni α2,3-sialyltransferase (CST-I); and porcine ST3Gal1 (pST3Gal1).

12. The plasmid of any one of claims 1-11, wherein the α-2,6-sialyltransferase is selected from: hST6GalNAc2; and hST6GalNAc4.

13. The plasmid of any one of claims 1-12, wherein the plasmid encodes enzymes selected from one or more amino acid sequences as set forth in SEQ ID NO: 1-52, or an amino acid sequence having at least 90% sequence identity thereto, provided that the enzymes retain their enzymatic activity.

14. The plasmid of any one of claims 1-12, wherein the plasmid has the DNA sequence set out in one of SEQ ID NO: 60-62, or a nucleic acid sequence having at least 90% sequence identity thereto, provided that the enzymes encoded by the sequence retain their enzymatic activity.

15. The plasmid of any one of claims 1-14, further comprising a second plasmid, wherein the second plasmid comprises a DNA sequence encoding:
(a) a hydrolysing UDP-GlcNAc 2′ epimerase;
(b) a sialic acid synthetase; and
(c) a CMP-NeuAc synthetase.

16. The plasmid of claim 15, wherein the second plasmid comprises at least 1 operon, wherein the DNA encoded in operon 3 comprises:
(i) at least 1 promoter;
(ii) a hydrolysing UDP-GlcNAc 2′ epimerase;
(iii) a sialic acid synthetase; and
(iv) a CMP-NeuAc synthetase.

17. The plasmid of claim 15 or 16, further comprising a ribosomal binding site encoded upstream of the start codon of each encoded gene.

18. The plasmid of claim 16, wherein the promoter in operon 3 is selected from: an inducible promoter and a constitutive promoter.

19. The plasmid of claim 16 or 17, wherein there are three copies of the promoter in operon

3.

20. The plasmid of any one of claims 15-19, wherein operon 3 is a Neisseria meningitidis neuBCA operon.

21. The plasmid of any one of claims 15-20, wherein the plasmid encodes enzymes having an amino acid sequence or sequences as set forth as SEQ ID NO: 53-55, or an amino acid sequence having at least 90% sequence identity thereto, provided that the enzymes retain their enzymatic activity.

22. The plasmid of any one of claims 15-20, wherein the plasmid has the DNA sequence set out in SEQ ID NO: 63, or a nucleic acid sequence having at least 90 % sequence identity thereto, provided that the enzymes encoded by the sequence retain their enzymatic activity.

23. The plasmid of any one of claims 1-22, further comprising a third plasmid, wherein the third plasmid comprises a DNA sequence encoding a target gene for expression, O-glycosylation, and sialylation or disialylation.

24. A recombinant bacterial cell, the bacterial cell having an oxidizing environment and comprising a plasmid or plasmids of any one of claims 1-23.

25. A recombinant bacterial cell, the bacterial cell having an reducing environment and comprising a plasmid or plasmids of any one of claims 1-23, wherein the plasmids are co-expressed with hPDI-quiescin-sulfydryl oxidase fusion protein (hPDI-QSOXib).

26. A recombinant bacterial cell, wherein said bacterial cell provides an oxidizing environment and comprises a chromosome, wherein the chromosome comprises integrated DNA encoding:
(a) a polypeptide N-acetylgalactosaminyltransferase;
(b) a β-1,3-galactosyltransferase;
(c) an UDP-Glc/GlcNAc 4-epimerase;
(d) a disulfide bond isomerase;
(e) an α-2,3-sialyltransferase; and
(f) an α-2,6-sialyltransferase.

27. The bacterial cell of claim 26, wherein the bacteria expresses the integrated DNA encoding (a)-(f) under the control of at least 1 promoter.

28. The bacterial cell of claim 26 or 27, further comprising a ribosomal binding site encoded upstream of the start codon of each encoded gene.

29. The bacterial cell of claim 26, 27 or 28, wherein at least one promoter is selected from:
an inducible promoter and a constitutive promoter.

30. The bacterial cell of any one of claims 26-29, wherein the polypeptide N-acetylgalactosaminyltransferase is human polypeptide N-acetylgalactosaminyltransferase 2 (hppGalNAcT2).

31. The bacterial cell of any one of claims 26-30, wherein the β-1,3-galactosyltransferase is selected from: Campylobacter jejuni β-1,3-galactosyltransferase (CgtB); and Drosophila melanogaster C1GalT1 galactosyltransferase (DmC1GalT1).

32. The bacterial cell of any one of claims 26-31, wherein the UDP-Glc/GlcNAc 4-epimerase is Campylobacter jejuni UDP-Glc/GlcNAc 4-epimerase (Cj-Gne).

33. The bacterial cell of any one of claims 26-32, wherein the disulfide bond isomerase is selected from: human disulfide bond isomerase (hPDI); and E. coli disulfide bond isomerase (DsbC).

34. The bacterial cell of any one of claims 26-33, wherein theα-2,3-sialyltransferase is selected from: Campylobacter jejuni α2,3-sialyltransferase (CST-I); porcine ST3Gal1 (pST3Gal1); and human ST3Gal1.

35. The bacterial cell of any one of claims 26-34, wherein theα-2,6-sialyltransferase is selected from: hST6GalNAc2; and hST6GalNAc4.

36. The bacterial cell of any one of claims 26-35, wherein the chromosome encodes enzymes selected from one or more amino acid sequences as set forth in SEQ ID NOs:1-52, or an amino acid sequence having at least90% sequence identity thereto, provided that the enzymes retain their enzymatic activity.

37. The bacterial cell of any one of claims 26-35, wherein the chromosome has the DNA sequence set out in one of SEQ ID NOs:60-62, or a nucleic acid sequence having at least90% sequence identity thereto, provided that the enzymes encoded by the sequences retain their enzymatic activity.

38. The bacterial cell of any one of claims 26-37, the chromosome further comprising integrated DNA encoding:
(g) a hydrolysing UDP-GlcNAc 2′ epimerase;
(h) a sialic acid synthetase; and
(i) a CMP-NeuAc synthetase.

39. The bacterial cell of claim 38, wherein the bacteria expresses the further integrated DNA encoding (g)-(i) under the control of at least one promoter.

40. The bacterial cell of claim 38 or 39, further comprising a ribosomal binding site encoded upstream of the start codon of each encoded gene.

41. The bacterial cell of claim 39, wherein the at least one promoter is selected from: an inducible promoter and a constitutive promoter.

42. The bacterial cell of any one of claim 38-41, wherein operon 3 is a Neisseria meningitidis neuBCA operon.

43. The bacterial cell of any one of claims 38-42, wherein the chromosome encodes enzymes having an amino acid sequence as set forth as SEQ ID NOs: 53-55, or an amino acid sequence having at least 90% sequence identity thereto, provided that the enzymes retain their enzymatic activity.

44. The bacterial cell of any one of claims 38-42 wherein the chromosome has the DNA sequence set out in SEQ ID NO: 63, or a nucleic acid sequence having at least 90% sequence identity thereto, provided that the enzymes encoded by the sequences retain their enzymatic activity.

45. The bacterial cell of any one of claims 26-44, further comprising a DNA sequence encoding a target gene for expression, O-glycosylation, and sialylation or disialylation.

46. The bacterial cell of any one of claims 26-45, wherein the bacterial cell has been modified to reduce reductase activity.

47. A nucleic acid construct that directs expression in a prokaryotic cell, the nucleic acid construct comprising DNA encoding:
(a) a polypeptide N-acetylgalactosaminyltransferase;
(b) a β-1,3-galactosyltransferase;
(c) an UDP-Glc/GlcNAc 4-epimeraseUDP-Glc/GlcNAc 4-epimerase;
(d) a disulfide bond isomerase;
(e) an α-2,3-sialyltransferase;
(f) an α-2,6-sialyltransferase; and
(g) at least one promoter.

48. The nucleic acid construct of claim 47, further comprising a ribosomal binding site encoded upstream of the start codon of each encoded gene.

49. The nucleic acid construct of claim 47 or 48, wherein at least one promoter is selected from: an inducible promoter and a constitutive promoter. 5o. The nucleic acid construct of claim 47, 48 or 49, wherein the polypeptide N-acetylgalactosaminyltransferase is human polypeptide N-acetylgalactosaminyltransferase 2 (hppGalNAcT2).

51. The nucleic acid construct of any one of claims 47-50, wherein the β-1,3-galactosyltransferase is selected from: Campylobacter jejuni β-1,3-galactosyltransferase (CgtB); and Drosophila melanogaster C1GalT1 galactosyltransferase (DmC1GalT1).

52. The nucleic acid construct of any one of claims 47-51, wherein the UDP-Glc/GlcNAc 4-epimerase is Campylobacter jejuni UDP-Glc/GlcNAc 4-epimerase (Cj-Gne).

53. The nucleic acid construct of any one of claims 47-52, wherein the disulfide bond isomerase is selected from: human disulfide bond isomerase (hPDI); and E. coli disulfide bond isomerase (DsbC).

54. The nucleic acid construct of any one of claims 47-53, wherein theα-2,3-sialyltransferase is selected from: Campylobacter jejuni α2,3-sialyltransferase (CST-I); and porcine ST3Gal1 (pST3Gal1).

55. The nucleic acid construct of any one of claims 47-54, wherein the α-2,6-sialyltransferase is selected from; hST6GalNAc2; and hST6GalNAc4.

56. The nucleic acid construct of any one of claims 47-55, wherein the nucleic acid construct encodes enzymes selected from one or more amino acid sequences as set forth in SEQ ID NO: 1-52, or an amino acid sequence having at least 90% sequence identity thereto, provided that the enzymes retain their enzymatic activity.

57. The nucleic acid construct of any one of claims 47-55, wherein the nucleic acid construct has the DNA sequence set out in one of SEQ ID NO: 60-62, or a nucleic acid sequence having at least 90% sequence identity thereto, provided that the enzymes encoded by the sequences retain their enzymatic activity.

58. The nucleic acid construct of any one of claims 47-57, further comprising DNA encoding:
(h) at least one promoter;
(i) a hydrolysing UDP-GlcNAc 2′ epimerase;
(j) a sialic acid synthetase; and
(k) a CMP-NeuAc synthetase.

59. The nucleic acid construct of claim 58, further comprising a ribosomal binding site encoded upstream of the start codon of each encoded gene. 6o. The nucleic acid construct of claim 58 or 59, wherein the promoter is selected from: an inducible promoter and a constitutive promoter.

61. The nucleic acid construct of any one of claims 47-60, wherein the hydrolysing UDP-GlcNAc 2′ epimerase; a sialic acid synthetase; and the CMP-NeuAc synthetase may be a Neisseria meningitidis neuBCA operon.

62. The nucleic acid construct of any one of claims 58-61, wherein the nucleic acid construct encodes enzymes having an amino acid sequence as set forth as SEQ ID NO: 53-55, or an amino acid sequence having at least90% sequence identity thereto, provided that the enzymes retain their enzymatic activity.

63. The nucleic acid construct of any one of claims 58-61, wherein the nucleic acid construct has the DNA sequence set out in SEQ ID NO: 63, or a nucleic acid sequence having at least90% sequence identity thereto, provided that the enzymes encoded by the sequences retain their enzymatic activity.

64. The nucleic acid construct of any one of claims 47-63, further comprising DNA encoding a target gene for expression, O-glycosylation, and sialylation or disialylation.

65. The nucleic acid construct of any one of claims 47-64, wherein the nucleic acid construct resides in a bacterial cell.

66. The nucleic acid construct of claim 65, wherein the bacterial cell has been modified to provide an oxidizing environment within the cell cytoplasm.

67. The nucleic acid construct of claim 65 or 66, wherein the bacterial cell has been modified to reduce reductase activity.

68. A method for producing a sialylated or disialylated target protein in a bacterium, wherein the bacterium provides an oxidizing environment for posttranslational modification of expressed protein, the method comprising:
(a) expressing in the bacterium: a polypeptide N-acetylgalactosaminyltransferase; a β-1,3-galactosyltransferase; an UDP-G1c/G1cNAc 4-epimerase; a disulfide bond isomerase; an α-2,3-sialyltransferase; an α-2,6-sialyltransferase
(b) expressing in the bacterium: a hydrolysing UDP-GlcNAc 2′ epimerase; a sialic acid synthetase; and a CMP-NeuAc synthetase; and
(c) expressing in the bacterium a target protein for O-glycosylation and sialylation or disialylation.


SEQUENCE LISTING (108 sequences)

SEQ ID NO: 1 - protein, 913 aa, Homo sapiens
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFGSKKKDLHHSNGEEKAQSMETLPPGKVRWPDFNQEAYVGGTMVRSGQDPYARNKFNQVESDKLRMDRAIPDTRHDQCQRKQWRVDLPATSVVITFHNEARSALLRTVVSVLKKSPPHLIKEIILVDDYSNDPEDGALLGKIEKVRVLRNDRREGLMRSRVRGADAAQAKVLTFLDSHCECNEHWLEPLLERVAEDRTRVVSPIIDVINMDNFQYVGASADLKGGFDWNLVFKWDYMTPEQRRSRQGNPVAPIKTPMIAGGLFVMDKFYFEELGKYDMMMDVWGGENLEISFRVWQCGGSLEIIPCSRVGHVFRKQHPYTFPGGSGTVFARNTRRAAEVWMDEYKNFYYAAVPSARNVPYGNIQSRLELRKKLSCKPFKWYLENVYPELRVPDHQDIAFGALQQGTNCLDTLGHFADGVVGVYECHNAGGNQEWALTKEKSVKHMDLCLTVVDRAPGSLIKLQGCRENDSRQKWEQIEGNSKLRHVGSNLCLDSRTAKSGGLSVEVCGPALSQQWKFTLNLQQ

SEQ ID NO: 2 - protein, 571 aa, Homo sapiens
MRRRSRMLLCFAFLWVLGIAYYMYSGGGSALAGGAGGGAGRKEDWNEIDPIKKKDLHHSNGEEKAQSMETLPPGKVRWPDFNQEAYVGGTMVRSGQDPYARNKFNQVESDKLRMDRAIPDTRHDQCQRKQWRVDLPATSVVITFHNEARSALLRTVVSVLKKSPPHLIKEIILVDDYSNDPEDGALLGKIEKVRVLRNDRREGLMRSRVRGADAAQAKVLTFLDSHCECNEHWLEPLLERVAEDRTRVVSPIIDVINMDNFQYVGASADLKGGFDWNLVFKWDYMTPEQRRSRQGNPVAPIKTPMIAGGLFVMDKFYFEELGKYDMMMDVWGGENLEISFRVWQCGGSLEIIPCSRVGHVFRKQHPYTFPGGSGTVFARNTRRAAEVWMDEYKNFYYAAVPSARNVPYGNIQSRLELRKKLSCKPFKWYLENVYPELRVPDHQDIAFGALQQGTNCLDTLGHFADGVVGVYECHNAGGNQEWALTKEKSVKHMDLCLTVVDRAPGSLIKLQGCRENDSRQKWEQIEGNSKLRHVGSNLCLDSRTAKSGGLSVEVCGPALSQQWKFTLNLQQ

SEQ ID NO: 3 - protein, 559 aa, Homo sapiens
MRKFAYCKVVLATSLIWVLLDMFLLLYFSECNKCDEKKERGLPAGDVLEPVQKPHEGPGEMGKPVVIPKEDQEKMKEMFKINQFNLMASEMIALNRSLPDVRLEGCKTKVYPDNLPTTSVVIVFHNEAWSTLLRTVHSVINRSPRHMIEEIVLVDDASERDFLKRPLESYVKKLKVPVHVIRMEQRSGLIRARLKGAAVSKGQVITFLDAHCECTVGWLEPLLARIKHDRRTVVCPIIDVISDDTFEYMAGSDMTYGGFNWKLNFRWYPVPQREMDRRKGDRTLPVRTPTMAGGLFSIDRDYFQEIGTYDAGMDIWGGENLEISFRIWQCGGTLEIVTCSHVGHVFRKATPYTFPGGTGQIINKNNRRLAEVWMDEFKNFFYIISPGVTKVDYGDISSRVGLRHKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCNGSRSQQWLLRNVTLPEIF

SEQ ID NO: 4 - protein, 633 aa, Homo sapiens
MAHLKRLVKLHIKRHYHKKFWKLGAVIFFFIIVLVLMQREVSVQYSKEESRMERNMKNKNKMLDLMLEAVNNIKDAMPKMQIGAPVRQNIDAGERPCLQGYYTAAELKPVLDRPPQDSNAPGASGKAFKTTNLSVEEQKEKERGEAKHCFNAFASDRISLHRDLGPDTRPPECIEQKFKRCPPLPTTSVIIVFHNEAWSTLLRTVHSVLYSSPAILLKEIILVDDASVDEYLHDKLDEYVKQFSIVKIVRQRERKGLITARLLGATVATAETLTFLDAHCECFYGWLEPLLARIAENYTAVVSPDIASIDLNTFEFNKPSPYGSNHNRGNFDWSLSFGWESLPDHEKQRRKDETYPIKTPTFAGGLFSISKEYFEYIGSYDEEMEIWGGENIEMSFRVWQCGGQLEIMPCSVVGHVFRSKSPHSFPKGTQVIARNQVRLAEVWMDEYKEIFYRRNTDAAKIVKQKAFGDLSKRFEIKHRLQCKNFTWYLNNIYPEVYVPDLNPVISGYIKSVGQPLCLDVGENNQGGKPLIMYTCHGLGGNQYFEYSAQHEIRHNIQKELCLHAAQGLVQLKACTYKGHKTVVTGEQIWEIQKDQLLYNPFLKMCLSANGEHPSLVSCNPSDPLQKWILSQND

SEQ ID NO: 5 - protein, 578 aa, Homo sapiens
MAVRWTWAGKSCLLLAFLTVAYIFVELLVSTFHASAGAGRARELGSRRLSDLQKNTEDLSRPLYKKPPADSRALGEWGKASKLQLNEDELKQQEELIERYAINIYLSDRISLHRHIEDKRMYECKSQKFNYRTLPTTSVIIAFYNEAWSTLLRTIHSVLETSPAVLLKEIILVDDLSDRVYLKTQLETYISNLDRVRLIRTNKREGLVRARLIGATFATGDVLTFLDCHCECNSGWLEPLLERIGRDETAVVCPVIDTIDWNTFEFYMQIGEPMIGGFDWRLTFQWHSVPKQERDRRISRIDPIRSPTMAGGLFAVSKKYFQYLGTYDTGMEVWGGENLELSFRVWQCGGKLEIHPCSHVGHVFPKRAPYARPNFLQNTARAAEVWMDEYKEHFYNRNPPARKEAYGDISERKLLRERLRCKSFDWYLKNVFPNLHVPEDRPGWHGAIRSRGISSECLDYNSPDNNPTGANLSLFGCHGQGGNQFFEYTSNKEIRFNSVTELCAEVPEQKNYVGMQNCPKDGFPVPANIIWHFKEDGTIFHPHSGLCLSAYRTPEGRPDVQMRTCDALDKNQIWSFEK

SEQ ID NO: 6 - protein, 940 aa, Homo sapiens
MNRIRKFFRGSGRVLAFIFVASVIWLLFDMAALRLSFSEINTRVIKEDIVRRERIGFRVQPDQGKIFYSSIKEMKPPLRGHGKGAWGKENVRKTEESVLKVEVDLDQTQRERKMQNALGRGKVVPLWHPAHLQTLPVTPNKQKTDGRGTKPEASSHQGTPKQTTAQGAPKTSFIAAKGTQVVKISVHMGRVSLKQEPRKSHSPSSDTSKLAAERDLNVTISLSTDRPKQRSQAVANERAHPASTAVPKSGEAMALNKTKTQSKEVNANKHKANTSLPFPKFTVNSNRLRKQSINETPLGSLSKDDGARGAHGKKLNFSESHLVIITKEEEQKADPKEVSNSKTKTIFPKVLGKSQSKHISRNRSEMSSSSLAPHRVPLSQTNHALTGGLEPAKINITAKAPSTEYNQSHIKALLPEDSGTHQVLRIDVTLSPRDPKAPGQFGRPVVVPHGKEKEAERRWKEGNFNVYLSDLIPVDRAIEDTRPAGCAEQLVHNNLPTTSVIMCFVDEVWSTLLRSVHSVINRSPPHLIKEILLVDDFSTKDYLKDNLDKYMSQFPKVRILRLKERHGLIRARLAGAQNATGDVLTFLDSHVECNVGWLEPLLERVYLSRKKVACPVIEVINDKDMSYMTVDNFQRGIFVWPMNFGWRTIPPDVIAKNRIKETDTIRCPVMAGGLFSIDKSYFFELGTYDPGLDVWGGENMELSFKVWMCGGEIEIIPCSRVGHIFRNDNPYSFPKDRMKTVERNLVRVAEVWLDEYKELFYGHGDHLIDQGLDVGNLTQQRELRKKLKCKSFKWYLENVFPDLRAPIVRASGVLINVALGKCISIENTTVILEDCDGSKELQQFNYTWLRLIKCGEWCIAPIPDKGAVRLHPCDNRNKGLKWLHKSTSVFHPELVNHIVFENNQQLLCLEGNFSQKILKVAACDPVKPYQKWKFEKYYEA

SEQ ID NO: 7 - protein, 622 aa, Homo sapiens
MRLLRRRHMPLRLAMVGCAFVLFLFLLHRDVSSREEATEKPWLKSLVSRKDHVLDLMLEAMNNLRDSMPKLQIRAPEAQQTLFSINQSCLPGFYTPAELKPFWERPPQDPNAPGADGKAFQKSKWTPLETQEKEEGYKKHCFNAFASDRISLQRSLGPDTRPPECVDQKFRRCPPLATTSVIIVFHNEAWSTLLRTVYSVLHTTPAILLKEIILVDDASTEEHLKEKLEQYVKQLQVVRVVRQEERKGLITARLLGASVAQAEVLTFLDAHCECFHGWLEPLLARIAEDKTVVVSPDIVTIDLNTFEFAKPVQRGRVHSRGNFDWSLTFGWETLPPHEKQRRKDETYPIKSPTFAGGLFSISKSYFEHIGTYDNQMEIWGGENVEMSFRVWQCGGQLEIIPCSVVGHVFRTKSPHTFPKGTSVIARNQVRLAEVWMDSYKKIFYRRNLQAAKMAQEKSFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 8 - protein, 657 aa, Homo sapiens
MRLKIGFILRSLLVVGSFLGLVVLWSSLTPRPDDPSPLSRMREDRDVNDPMPNRGGNGLAPGEDRFKPVVPWPHVEGVEVDLESIRRINKAKNEQEHHAGGDSQKDIMQRQYLTFKPQTFTYHDPVLRPGILGNFEPKEPEPPGVVGGPGEKAKPLVLGPEFKQAIQASIKEFGFNMVASDMISLDRSVNDLRQEECKYWHYDENLLTSSVVIVFHNEGWSTLMRTVHSVIKRTPRKYLAEIVLIDDFSNKEHLKEKLDEYIKLWNGLVKVFRNERREGLIQARSIGAQKAKLGQVLIYLDAHCEVAVNWYAPLVAPISKDRTICTVPLIDVINGNTYEIIPQGGGDEDGYARGAWDWSMLWKRVPLTPQEKRLRKTKTEPYRSPAMAGGLFAIEREFFFELGLYDPGLQIWGGENFEISYKIWQCGGKLLFVPCSRVGHIYRLEGWQGNPPPIYVGSSPTLKNYVRVVEVWWDEYKDYFYASRPESQALPYGDISELKKFREDHNCKSFKWFMEEIAYDITSHYPLPPKNVDWGEIRGFETAYCIDSMGKTNGGFVELGPCHRMGGNQLFRINEANQLMQYDQCLTKGADGSKVMITHCNLNEFKEWQYFKNLHRFTHIPSGKCLDRSEVLHQVFISNCDSSKTTQKWEMNNIHSV

SEQ ID NO: 9 - protein, 637 aa, Homo sapiens
MMFWRKLPKALFIGLTLAIAVNLLLVFSSKGTLQNLFTGGLHRELPLHLNKRYGAVIKRLSHLEVELQDLKESMKLALRQQENVNSTLKRAKDEVRPLLKAMETKVNETKKHKTQMKLFPHSQLFRQWGEDLSEAQQKAAQDLFRKFGYNAYLSNQLPLNRTIPDTRDYRCLRKTYPSQLPSLSVILIFVNEALSIIQRAITSIINRTPSRLLKEIILVDDFSSNGELKVHLDEKIKLYNQKYPGLLKIIRHPERKGLAQARNTGWEAATADVVAILDAHIEVNVGWAEPILARIQEDRTVIVSPVFDNIRFDTFKLDKYELAVDGFNWELWCRYDALPQAWIDLHDVTAPVKSPSIMGILAANRHFLGEIGSLDGGMLIYGGENVELSLRVWQCGGKVEILPCSRIAHLERHHKPYALDLTAALKRNALRVAEIWMDEHKHMVYLAWNIPLQNSGIDFGDVSSRMALREKLKCKTFDWYLKNVYPLLKPLHTIVGYGRMKNLLDENVCLDQGPVPGNTPIMYYCHEFSSQNVYYHLTGELYVGQLIAEASASDRCLTDPGKAEKPTLEPCSKAAKNRLHIYWDFKPGGAVINRDTKRCLEMKKDLLGSHVLVLQTCSTQVWEIQHTVRDWGQTNSQ

SEQ ID NO: 10 - protein, 603 aa, Homo sapiens
MAVARKIRTLLTVNILVFVGIVLFSVYCRLQGRSQELVRIVSGDRRVRSRHAKVGTLGDREAILQRLDHLEEVVYNQLNGLAKPIGLVEGPGGLGQGGLAATLRDDGQEAEGKYEEYGYNAQLSDRISLDRSIPDYRPRKCRQMSYAQDLPQVSVVFIFVNEALSVILRSVHSVVNHTPSQLLKEVILVDDNSDNVELKFNLDQYVNKRYPGLVKIVRNSRREGLIRARLQGWKAATAPVVGFFDAHVEFNTGWAEPALSRIREDRRRIVLPAIDNIKYSTFEVQQYANAAHGYNWGLRCMYIIPPQDWLDRGDESAPIRTPAMIGCSFVVDREYFGDIGLLDPGMEVYGGENVELGMRVWQCGGSMEVLPCSRVAHIERTRKPYNNDIDYYAKRNALRAAEVWMDDFKSHVYMAWNIPMSNPGVDFGDVSERLALRQRLKCRSFKWYLENVYPEMRVYNNTLTYGEVRNSKASAYCLDQGAEDGDRAILYPCHGMSSQLVRYSADGLLQLGPLGSTAFLPDSKCLVDDGTGRMPTLKKCEDVARPTQRLWDFTQSGPIVSRATGRCLEVEMSKDANFGLRLVVQRCSGQKWMIRNWIKHARH

SEQ ID NO: 11 - protein, 603 aa, Homo sapiens
MRRKEKRLLQAVALVLAALVLLPNVGLWALYRERQPDGTPGGSGAAVAPAAGQGSHSRQKKTFFLGDGQKLKDWHDKEAIRRDAQRVGNGEQGRPYPMTDAERVDQAYRENGFNIYVSDKISLNRSLPDIRHPNCNSKRYLETLPNTSIIIPFHNEGWSSLLRTVHSVLNRSPPELVAEIVLVDDFSDREHLKKPLEDYMALFPSVRILRTKKREGLIRTRMLGASVATGDVITFLDSHCEANVNWLPPLLDRIARNRKTIVCPMIDVIDHDDFRYETQAGDAMRGAFDWEMYYKRIPIPPELQKADPSDPFESPVMAGGLFAVDRKWFWELGGYDPGLEIWGGEQYEISFKVWMCGGRMEDIPCSRVGHIYRKYVPYKVPAGVSLARNLKRVAEVWMDEYAEYIYQRRPEYRHLSAGDVAVQKKLRSSLNCKSFKWFMTKIAWDLPKFYPPVEPPAAAWGEIRNVGTGLCADTKHGALGSPLRLEGCVRGRGEAAWNNMQVFTFTWREDIRPGDPQHTKKFCFDAISHTSPVTLYDCHSMKGNQLWKYRKDKTLYHPVSGSCMDCSESDHRIFMNTCNPSSLTQQWLFEHTNSTVLEKFNRN

SEQ ID NO: 12 - protein, 608 aa, Homo sapiens
MGSVTVRYFCYGCLFTSATWTVLLFVYFNFSEVTQPLKNVPVKGSGPHGPSPKKFYPRFTRGPSRVLEPQFKANKIDDVIDSRVEDPEEGHLKFSSELGMIFNERDQELRDLGYQKHAFNMLISDRLGYHRDVPDTRNAACKEKFYPPDLPAASVVICFYNEAFSALLRTVHSVIDRTPAHLLHEIILVDDDSDFDDLKGELDEYVQKYLPGKIKVIRNTKREGLIRGRMIGAAHATGEVLVFLDSHCEVNVMWLQPLLAAIREDRHTVVCPVIDIISADTLAYSSSPVVRGGFNWGLHFKWDLVPLSELGRAEGATAPIKSPTMAGGLFAMNRQYFHELGQYDSGMDIWGGENLEISFRIWMCGGKLFIIPCSRVGHIFRKRRPYGSPEGQDTMTHNSLRLAHVWLDEYKEQYFSLRPDLKTKSYGNISERVELRKKLGCKSFKWYLDNVYPEMQISGSHAKPQQPIFVNRGPKRPKVLQRGRLYHLQTNKCLVAQGRPSQKGGLVVLKACDYSDPNQIWIYNEEHELVLNSLLCLDMSETRSSDPPRLMKCHGSGGSQQWTFGKNNRLYQVSVGQCLRAVDPLGQKGSVAMAICDGSSSQQWHLEG

SEQ ID NO: 13 - protein, 581 aa, Homo sapiens [sequence omitted]

SEQ ID NO: 14 - protein, 556 aa, Homo sapiens
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRTPTMAGGLFSIDRNYFEEIGTYDAGMDIWGGENLEMSFRIWQCGGSLEIVTCSHVGHVFRKATPYTFPGGTGHVINKNNRRLAEVWMDEFKDFFYIISPGVVKVDYGDVSVRKTLRENLKCKPFSWYLENIYPDSQIPRRYYSLGEIRNVETNQCLDNMGRKENEKVGIFNCHGMGGNQVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 15 - protein, 552 aa, Homo sapiens
MRRLTRRLVLPVFGVLWITVLLFFWVTKRKLEVPTGPEVQTPKPSDADWDDLWDQFDERRYLNAKKWRVGDDPYKLYAFNQRESERISSNRAIPDTRHLRCTLLVYCTDLPPTSIIITFHNEARSTLLRTIRSVLNRTPTHLIREIILVDDFSNDPDDCKQLIKLPKVKCLRNNERQGLVRSRIRGADIAQGTTLTFLDSHCEVNRDWLQPLLHRVKEDYTRVVCPVIDIINLDTFTYIESASELRGGFDWSLHFQWEQLSPEQKARRLDPTEPIRTPIIAGGLFVIDKAWFDYLGKYDMDMDIWGGENFEISFRVWMCGGSLEIVPCSRVGHVFRKKHPYVFPDGNANTYIKNTKRTAEVWMDEYKQYYYAARPFALERPFGNVESRLDLRKNLRCQSFKWYLENIYPELSIPKESSIQKGNIRQRQKCLESQRQNNQETPNLKLSPCAKVKGEDAKSQVWAFTYTQQILQEELCLSVITLFPGAPVVLVLCKNGDDRQQWTKTGSHIEHIASHLCLDTDMFGDGTENGKEIVVNPCESSLMSQHWDMVSS

SEQ ID NO: 16 - protein, 639 aa, Homo sapiens
MLLRKRYRHRPCRLQFLLLLLMLGCVLMMVAMLHPPHHTLHQTVTAQASKHSPEARYRLDFGESQDWVLEAEDEGEEYSPLEGLPPFISLREDQLLVAVALPQARRNQSQGRRGGSYRLIKQPRRQDKEAPKRDWGADEDGEVSEEEELTPFSLDPRGLQEALSARIPLQRALPEVRHPLCLQQHPQDSLPTASVILCFHDEAWSTLLRTVHSILDTVPRAFLKEIILVDDLSQQGQLKSALSEYVARLEGVKLLRSNKRLGAIRARMLGATRATGDVLVFMDAHCECHPGWLEPLLSRIAGDRSRVVSPVIDVIDWKTFQYYPSKDLQRGVLDWKLDFHWEPLPEHVRKALQSPISPIRSPVVPGEVVAMDRHYFQNTGAYDSLMSLRGGENLELSFKAWLCGGSVEILPCSRVGHIYQNQDSHSPLDQEATLRNRVRIAETWLGSFKETFYKHSPEAFSLSKAEKPDCMERLQLQRRLGCRTFHWFLANVYPELYPSEPRPSFSGKLHNTGLGLCADCQAEGDILGCPMVLAPCSDSRQQQYLQHTSRKEIHFGSPQHLCFAVRQEQVILQNCTEEGLAIHQQHWDFQENGMIVHILSGKCMEAVVQENNKDLYLRPCDGKARQQWRFDQINAVDER

SEQ ID NO: 17 - protein, 558 aa, Homo sapiens
MRKIRANAIAILTVAWILGTFYYLWQDNRAHAASSGGRGAQRAGRRSEQLREDRTIPLIVTGTPSKGFDEKAYLSAKQLKAGEDPYRQHAFNQLESDKLSPDRPIRDTRHYSCPSVSYSSDLPATSVIITFHNEARSTLLRTVKSVLNRTPANLIQEIILVDDFSSDPEDCLLLTRIPKVKCLRNDRREGLIRSRVRGADVAAATVLTFLDSHCEVNTEWLPPMLQRVKEDHTRVVSPIIDVISLDNFAYLAASADLRGGFDWSLHFKWEQIPLEQKMTRTDPTRPIRTPVIAGGIFVIDKSWFNHLGKYDAQMDIWGGENFELSFRVWMCGGSLEIVPCSRVGHVFRKRHPYNFPEGNALTYIRNTKRTAEVWMDEYKQYYYEARPSAIGKAFGSVATRIEQRKKMNCKSFRWYLENVYPELTVPVKEALPGIIKQGVNCLESQGQNTAGDFLLGMGICRGSAKNPQPAQAWLFSDHLIQQQGKCLAATSTLMSSPGSPVILQMCNPREGKQKWRRKGSFIQHSVSGLCLETKPAQLVTSKCQADAQAQQWQLLPHT

SEQ ID NO: 18 - protein, 598 aa, Homo sapiens
MASLRRVKVLLVLNLIAVAGFVLFLAKCRPIAVRSGDAFHEIRPRAEVANLSAHSASPIQDAVLKRLSLLEDIVYRQLNGLSKSLGLIEGYGGRGKGGLPATLSPAEEEKAKGPHEKYGYNSYLSEKISLDRSIPDYRPTKCKELKYSKDLPQISIIFIFVNEALSVILRSVHSAVNHTPTHLLKEIILVDDNSDEEELKVPLEEYVHKRYPGLVKVVRNQKREGLIRARIEGWKVATGQVTGFFDAHVEFTAGWAEPVLSRIQENRKRVILPSIDNIKQDNFEVQRYENSAHGYSWELWCMYISPPKDWWDAGDPSLPIRTPAMIGCSFVVNRKFFGEIGLLDPGMDVYGGENIELGIKVWLCGGSMEVLPCSRVAHIERKKKPYNSNIGFYTKRNALRVAEVWMDDYKSHVYIAWNLPLENPGIDIGDVSERRALRKSLKCKNFQWYLDHVYPEMRRYNNTVAYGELRNNKAKDVCLDQGPLENHTAILYPCHGWGPQLARYTKEGFLHLGALGTTTLLPDTRCLVDNSKSRLPQLLDCDKVKSSLYKRWNFIQNGAIMNKGTGRCLEVENRGLAGIDLILRSCTGQRWTIKNSIK

SEQ ID NO: 19 - protein, 607 aa, Homo sapiens
MVCTRKTKTLVSTCVILSGMTNIICLLYVGWVTNYIASVYVRGQEPAPDKKLEEDKGDTLKIIERLDHLENVIKQHIQEAPAKPEEAEAEPFTDSSLFAHWGQELSPEGRRVALKQFQYYGYNAYLSDRLPLDRPLPDLRPSGCRNLSFPDSLPEVSIVFIFVNEALSVLLRSIHSAMERTPPHLLKEIILVDDNSSNEELKEKLTEYVDKVNSQKPGFIKVVRHSKQEGLIRSRVSGWRAATAPVVALFDAHVEFNVGWAEPVLTRIKENRKRIISPSFDNIKYDNFEIEEYPLAAQGFDWELWCRYLNPPKAWWKLENSTAPIRSPALIGCFIVDRQYFQEIGLLDEGMEVYGGENVELGIRVWQCGGSVEVLPCSRIAHIERAHKPYTEDLTAHVRRNALRVAEVWMDEFKSHVYMAWNIPQEDSGIDIGDITARKALRKQLQCKTFRWYLVSVYPEMRMYSDIIAYGVLQNSLKTDLCLDQGPDTENVPIMYICHGMTPQNVYYTSSQQIHVGILSPTVDDDDNRCLVDVNSRPRLIECSYAKAKRMKLHWQFSQGGPIQNRKSKRCLELQENSDLEFGFQLVLQKCSGQHWSITNVLRSLAS

SEQ ID NO: 20 - protein, 601 aa, Homo sapiens
MKRKQKRFLQMTLLFTVALIFLPNVGLWSLYKDKHLVKSAEPGEQQTFPLGLGDGQFYSWTDGLRRKDWHDYESIQKEAMRSGKGEHGKPYPLTEEDHDDSAYRENGFNIFVSNNIALERSLPDIRHANCKHKMYLERLPNTSIIIPFHNEGWTSLLRTIHSIINRTPGSLIAEIILVDDFSEREHLKDKLEEYMARFSKVRIVRTKKREGLIRTRLLGASMARGEVLTFLDSHCEVNVNWLPPLLNQIALNHKTIVCPMIDVIDHNHFGYEAQAGDAMRGAFDWEMYYKRIPIPPELQRADPSDPFESPVMAGGLFAVDRKWFWELGGYDPGLEIWGGEQYEISFKVWMCGGEMFDVPCSRVGHIYRKYVPYKVPSGTSLARNLKRVAETWMDEFAEYIYQRRPEYRHLSTGDISAQKELRKQLKCKDFKWFMAAVAWDVPKYYPPVEPPPAAWGEIRNVAANLCVDSKHGATGTELRLDICVKDGSERTWSHEQLFTFGWREDIRPGEPLHTRKFCFDAISHNSPVTLYDCHGMKGNQLWGYRKDRTLFHPVSNSCMDCNPAEKKIFMARCDPLSETQQWIFEHINMTVLEKFNHHANS

SEQ ID NO: 21 - protein, 271 aa, Campylobacter jejuni
FKISIILPTYNVEQYIARAIESCINQTFKDIEIIVVDDCGNDNSINIAKEYSKKDKRIKIIHNEKNLGLLRARYEGVKVANSPYIMFLDPDDYLELNACEECIKILDEQDEVDLVFFNAIVESNVISYKKFDFNSGFYSKKEFVKKIIAKKNLYWTMWGKLIRKKLYLEAFASLRLEKDVKINMAEDVLLYYPMLSQAQKIAYMNCNLYHYVPNNNSICNTKNEVLVKNNIQELQLVLNYLRQNYILNKYCSVLYVLIKYLLYIQIYKIKP

SEQ ID NO: 22 - protein, 388 aa, Drosophila melanogaster
MTANSLLGRSILNEGRSNKRSFVSLIVGLIVGFCLAELFVYSTPERSEFMPYDGHRHGDVNDAHHSHDMMEMSGPEQDVGGHEHVHENSTIAERLYSEVRVLCWIMTNPSNHQKKARHVKRTWGKRCNKLIFMSSAKDDELDAVALPVGEGRNNLWGKTKEAYKYIYEHHINDADWFLKADDDTYTIVENMRYMLYPYSPETPVYFGCKFKPYVKQGYMSGGAGYVLSREAVRRFVVEALPNPKLCKSDNSGAEDVEIGKCLQNVNVLAGDSRDSNGRGRFFPFVPEHHLIPSHTDKKFWYWQYIFYKTDEGLDCCSDNAISFHYVSPNQMYVLDYLIYHLRPYGIINTPDALPNKLAVGELMPEIKEQATESTSDGVSKRSAETKTQ

SEQ ID NO: 23 - protein, 343 aa, Drosophila melanogaster
MSEFMPYDGHRHGDVNDAHHSHDMMEMSGPEQDVGGHEHVHENSTIAERLYSEVRVLCWIMTNPSNHQKKARHVKRTWGKRCNKLIFMSSAKDDELDAVALPVGEGRNNLWGKTKEAYKYIYEHHINDADWFLKADDDTYTIVENMRYMLYPYSPETPVYFGCKFKPYVKQGYMSGGAGYVLSREAVRRFVVEALPNPKLCKSDNSGAEDVEIGKCLQNVNVLAGDSRDSNGRGRFFPFVPEHHLIPSHTDKKFWYWQYIFYKTDEGLDCCSDNAISFHYVSPNQMYVLDYLIYHLRPYGIINTPDALPNKLAVGELMPEIKEQATESTSDGVSKRSAETKTQ

SEQ ID NO: 24 - protein, 379 aa, Homo sapiens
MSRSGTDPQQRQQASEADAAAATFRANDHQHIRYNPLQDEWVLVSAHRMKRPWQGQVEPQLLKTVPRHDPLNPLCPGAIRANGEVNPQYDSTFLFDNDFPALQPDAPSPGPSDHPLFQAKSARGVCKVMCFHPWSDVTLPLMSVPEIRAVVDAWASVTEELGAQYPWVQIFENKGAMMGCSNPHPHCQVWASSFLPDIAQREERSQQAYKSQHGEPLLMEYSRQELLRKERLVLTSEHWLVLVPFWATWPYQTLLLPRRHVRRLPELTPAERDDLASIMKKLLTKYDNLFETSFPYSMGWHGAPTGSEAGANWNHWQLHAHYYPPLLRSATVRKFMVGYEMLAQAQRDLTPEQAAERLRALPEVHYHLGQKDRETATIA

SEQ ID NO: 25 - protein, 328 aa, Campylobacter jejuni
MKILISGGAGYIGSHTLRQFLKTDHEICVLDNLSKGSKIAIEDLQKTRAFKFFEQDLSDFQGVKALFEREKFDAIVHFAASIEVFESMQNPLKYYMNNTVNTTNLIETCLQTGVNKFIFSSTAATYGEPQTPVVSETSPLAPINPYGRSKLMSEEVLRDASMANPEFKHCILRYFNVAGACMDYTLGQRYPKATLLIKVAAECAAGKRDKLFIFGDDYDTKDGTCIRDFIHVDDISSAHLAALDYLKENESNVFNVGYGHGFSVKEVIEAMKKVSGVDFKVELAPRRAGDPSVLISDASKIRNLTSWQPKYDDLELICKSAFDWEKQC

SEQ ID NO: 26 - protein, 328 aa, Campylobacter jejuni; identical to SEQ ID NO: 25

SEQ ID NO: 27 - protein, 348 aa, Homo sapiens
MAEKVLVTGGAGYIGSHTVLELLEAGYLPVVIDNFHNAFRGGGSLPESLRRVQELTGRSVEFEEMDILDQGALQRLFKKYSFMAVIHFAGLKAVGESVQKPLDYYRVNLTGTIQLLEIMKAHGVKNLVFSSSATVYGNPQYLPLDEAHPTGGCTNPYGKSKFFIEEMIRDLCQADKTWNAVLLRYFNPTGAHASGCIGEDPQGIPNNLMPYVSQVAIGRREALNVFGNDYDTEDGTGVRDYIHVVDLAKGHIAALRKLKEQCGCRIYNLGTGTGYSVLQMVQAMEKASGKKIPYKVVARREGDVAACYANPSLAQEELGWTAALGLDRMCEDLWRWQKQNPSGFGTQA

SEQ ID NO: 28 - protein, 347 aa, Mus musculus
MEKVLVTGGAGYIGSHTVLELLEAGYSPVVIDNFHNAIRGEDSMPESLRRVQELTGRSVEFEEMDILDQAALQHLFKKHSFKAVIHFAGLKAVGESVQKPLDYYRVNLTGTIQLLEIMRAHGVKNLVFSSSATVYGNPQYLPLDEAHPTGGCTNPYGKSKFFIEEMIRDLCRADTAWNAVLLRYFNPIGAHASGRIGEDPQGIPNNLMPYVSQVAIGRREALNVFGDDYATEDGTGVRDYIHVVDLAKGHIAALKKLKEQCGCRTYNLGTGTGYSVLQMVQAMEKASGKKIPYKVVARREGDVAACYANPSLAHEELGWTAALGLDRMCEDLWRWQKQNPSGFGAQA

SEQ ID NO: 29 - protein, 338 aa, Escherichia coli
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 30 - protein, 216 aa, Escherichia coli
DDAAIQQTLAKMGIKSSDIQPAPVAGMKTVLTNSGVLYITDDGKHIIQGPMYDVSGTAPVNVTNKMLLKQLNALEKEMIVYKAPQEKHVITVFTDITCGYCHKLHEQMADYNALGITVRYLAFPRQGLDSDAEKEMKAIWCAKDKNKAFDDVMAGKSVAPASCDVDIADHYALGVQLGVSGTPAVVLSNGTLVPGYQPPKEMKEFLDEHQKMTSGK

SEQ ID NO: 31 - protein, 519 aa, Homo sapiens
MARAGPAWLLLAIWVVLPSWLSSAKVSSLIERISDPKDLKKLLRTRNNVLVLYSKSEVAAENHLRLLSTVAQAVKGQGTICWVDCGDAESRKLCKKMKVDLSPKDKKVELFHYQDGAFHTEYNRAVTFKSIVAFLKDPKGPPLWEEDPGAKDVVHLDSEKDFRRLLKKEEKPLLIMFYAPWCSMCKRMMPHFQKAATQLRGHAVLAGMNVYSSEFENIKEEYSVRGFPTICYFEKGRFLFQYDNYGSTAEDIVEWLKNPQPPQPQVPETPWADEGGSVYHLTDEDFDQFVKEHSSVLVMFHAPWCGHCKKMKPEFEKAAEALHGEADSSGVLAAVDATVNKALAERFHISEFPTLKYFKNGEKYAVPVLRTKKKFLEWMQNPEAPPPPEPTWEEQQTSVLHLVGDNFRETLKKKKHTLVMFYAPWCPHCKKVIPHFTATADAFKDDRKIACAAVDCVKDKNQDLCQQEAVKGYPTFHYYHYGKFAEKYDSDRTELGFTNYIRALREGDHERLGKKKEEL

SEQ ID NO: 32 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 33 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 34 - protein, 645 aa, Homo sapiens
MRPRKAFLLLLLLGLVQLLAVAGAEGPDEDSSNRENAIEDEEEEEEEDDDEEEDDLEVKEENGVLVLNDANFDNFVADKDTVLLEFYAPWCGHCKQFAPEYEKIANILKDKDPPIPVAKIDATSASVLASRFDVSGYPTIKILKKGQAVDYEGSRTQEEIVAKVREVSQPDWTPPPEVTLVLTKENFDEVVNDADIILVEFYAPWCGHCKKLAPEYEKAAKELSKRSPPIPLAKVDATAETDLAKRFDVSGYPTLKIFRKGRPYDYNGPREKYGIVDYMIEQSGPPSKEILTLKQVQEFLKDGDDVIIIGVFKGESDPAYQQYQDAANNLREDYKFHHTFSTEIAKFLKVSQGQLVVMQPEKFQSKYEPRSHMMDVQGSTQDSAIKDFVLKYALPLVGHRKVSNDAKRYTRRPLVVVYYSVDFSFDYRAATQFWRSKVLEVAKDFPEYTFAIADEEDYAGEVKDLGLSESGEDVNAAILDESGKKFAMEPEEFDSDTLREFVTAFKKGKLKPVIKSQPVPKNNKGPVKVVVGKTFDSIVMDPKKDVLIEFYAPWCGHCKQLEPVYNSLAKKYKGQKGLVIAKMDATANDVPSDRYKVEGFPTIYFAPSGDKKNPVKFEGGDRDLEHLSKFIEEHATKLSRTKEEL

SEQ ID NO: 35 - protein, 236 aa, Escherichia coli
MKKGFMLFTLLAAFSGFAQADDAAIQQTLAKMGIKSSDIQPAPVAGMKTVLTNSGVLYITDDGKHIIQGPMYDVSGTAPVNVTNKMLLKQLNALEKEMIVYKAPQEKHVITVFTDITCGYCHKLHEQMADYNALGITVRYLAFPRQGLDSDAEKEMKAIWCAKDKNKAFDDVMAGKSVAPASCDVDIADHYALGVQLGVSGTPAVVLSNGTLVPGYQPPKEMKEFLDEHQKMTSGK

SEQ ID NO: 36 - protein, 1179 aa, Artificial Sequence
  APO-3 hPDI-quiescin-sulfydryl oxidase fusion protein (hPDI-QSOX1b)
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMDAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDELPTPTPTTPTPTPTTAPRSALYSPSDPLTLLQADTVRGAVLGSRSAWAVEFFASWCGHCIAFAPTWKALAEDVKAWRPALYLAALDCAEETNSAVCRDFNIPGFPTVRFFKAFTKNGSGAVFPVAGADVQTLRERLIDALESHHDTWPPACPPLEPAKLEEIDGFFARNNEEYLALIFEKGGSYLGREVALDLSQHKGVAVRRVLNTEANVVRKFGVTDFPSCYLLFRNGSVSRVPVLMESRSFYTAYLQRLSGLTREAAQTTVAPTTANKIAPTVWKLADRSKIYMADLESALHYILRIEVGRFPVLEGQRLVALKKFVAVLAKYFPGRPLVQNFLHSVNEWLKRQKRNKIPYSFFKTALDDRKEGAVLAKKVNWIGCQGSEPHFRGFPCSLWVLFHFLTVQAARQNVDHSQEAAKAKEVLPAIRGYVHYFFGCRDCASHFEQMAAASMHRVGSPNAAVLWLWSSHNRVNARLAGAPSEDPQFPKVQWPPRELCSACHNERLDVPVWDVEATLNFLKAHFSPSNIILDFPAAGSAARRDVQNVAAAPELAMGALELESRNSTLDPGKPEMMKSPTNTTPHVPAEGPELI

SEQ ID NO: 37 - protein, 300 aa, Campylobacter jejuni
MTRTRMENELIVSKNMQNIIIAGNGPSLKNINYKRLPREYDVFRCNQFYFEDKYYLGKKIKAVFFNPGVFLQQYHTAKQLILKNEYEIKNIFCSTFNLPFIESNDFLHQFYNFFPDAKLGYEVIENLKEFYAYIKYNEIYFNKRITSGVYMCAIAIALGYKTIYLCGIDFYEGDVIYPFEAMSTNIKTIFPGIKDFKPSNCHSKEYDIEALKLLKSIYKVNIYALCDDSILANHFPLSININNNFTLENKHNNSINDILLTDNTPGVSFYKNQLKADNKIMLNFYNILHSKDNLIKFLNK

SEQ ID NO: 38 - protein, 285 aa, Sus scrofa domesticus
MRPCTCTRCIEEQRVSAWFDERFNRSMQPLLTAKNAHLEEDTYKWWLRLQREKQPNNLNDTIRELFQVVPGNVDPLLEKRLVSCRRCAVVGNSGNLKESYYGPQIDSHDFVLRMNKAPTEGFEADVGSKTTHHFVYPESFRELAQEVSMILVPFKTTDLEWVISATTTGRISHTYVPVPAKIKVKKEKILIYHPAFIKYVFDRWLQGHGRYPSTGILSVIFSLHICDEVDLYGFGADSKGNWHHYWENNPSAGAFRKTGVHDGDFESNVTTILASINKIRIFKGR

SEQ ID NO: 39 - protein, 236 aa, Campylobacter jejuni; identical to SEQ ID NO: 35

SEQ ID NO: 40 - protein, 340 aa, Homo sapiens
MVTLRKRTLKVLTFLVLFIFLTSFFLNYSHTMVATTWFPKQMVLELSENLKRLIKHRPCTCTHCIGQRKLSAWFDERFNQTMQPLLTAQNALLEDDTYRWWLRLQREKKPNNLNDTIKELFRVVPGNVDPMLEKRSVGCRRCAVVGNSGNLRESSYGPEIDSHDFVLRMNKAPTAGFEADVGTKTTHHLVYPESFRELGDNVSMILVPFKTIDLEWVVSAITTGTISHTYIPVPAKIRVKQDKILIYHPAFIKYVFDNWLQGHGRYPSTGILSVIFSMHVCDEVDLYGFGADSKGNWHHYWENNPSAGAFRKTGVHDADFESNVTATLASINKIRIFKGR

SEQ ID NO: 41 - protein, 290 aa, Campylobacter jejuni
MKKVIIAGNGPSLKEIDYSRLPNDFDVFRCNQFYFEDKYYLGKKCKAVFYNPILFFEQYYTLKHLIQNQEYETELIMCSNYNQAHLENENFVKTFYDYFPDAHLGYDFFKQLKDFNAYFKFHEIYFNQRITSGVYMCAVAIALGYKEIYLSGIDFYQNGSSYAFDTKQKNLLKLAPNFKNDNSHYIGHSKNTDIKALEFLEKTYKIKLYCLCPNSLLANFIELAPNLNSNFIIQEKNNYTKDILIPSSEAYGKFSKNINFKKIKIKENIYYKLIKDLLRLPSDIKHYFKG

SEQ ID NO: 42 - protein, 260 aa, Campylobacter jejuni
MKKVIIAGNGPSLKEIDYSRLPNDFDVFRCNQFYFEDKYYLGKKCKAVFYNPSLFFEQYYTLKHLIQNQEYETELIMCSNYNQAHLENENFVKTFYDYFPDAHLGYDFFKQLKDFNAYFKFHEIYFNQRITSGVYMCAVAIALGYKEIYLSGIDFYQNGSSYAFDTKQKNLLKLAPNFKNDNSHYIGHSKNTDIKALEFLEKTYKIKLYCLCPNSLLANFIELAPNLNSNFIIQEKNNYTKDILIPSSEAYGKFSKNINF

SEQ ID NO: 43 - protein, 322 aa, Homo sapiens
SKASNSWTGKGQACRHLLHLAIQRHPHFRGLFNLSIPVLLWGDLFTPALWDRLSQHKAPYGWRGLSHQVIASTLSLLNGSESAKLFAPPRDTPPKCIRCAVVGNGGILNGSRQGPNIDAHDYVFRLNGAVIKGFERDVGTKTSFYGFTVNTMKNSLVSYWNLGFTSVPQGQDLQYIFIPSDIRDYVMLRSAILGVPVPEGLDKGDRPHAYFGPEASASKFKLLHPDFISYLTERFLKSKLINTHFGDLYMPSTGALMLLTALHTCDQVSAYGFITSNYWKFSDHYFERKMKPLIFYANHDLSLEAALWRDLHKAGILQLYQR

SEQ ID NO: 44 - protein, 268 aa, Homo sapiens
MTCLDHHFPTGSRPTVPGPLHFSGYSSVPDGKPLVREPCRSCAVVSSSGQMLGSGLGAEIDSAECVFRMNQAPTVGFEADVGQRSTLRVVSHTSVPLLLRNYSHYFQKARDTLYMVWGQGRHMDRVLGGRTYRTLLQLTRMYPGLQVYTFTERMMAYCDQIFQDETGKNRRQSGSFLSTGWFTMILALELCEEIVVYGMVSDSYCREKSHPSVPYHYFEKGRLDECQMYLAHEQAPRSAHRFITEKAVFSRWAKKRPIVFAHPSWRTE

SEQ ID NO: 45 - protein, 374 aa, Homo sapiens
MGLPRGSFFWLLLLLTAACSGLLFALYFSAVQRYPGPAAGARDTTSFEAFFQSKASNSWTGKGQACRHLLHLAIQRHPHFRGLFNLSIPVLLWGDLFTPALWDRLSQHKAPYGWRGLSHQVIASTLSLLNGSESAKLFAPPRDTPPKCIRCAVVGNGGILNGSRQGPNIDAHDYVFRLNGAVIKGFERDVGTKTSFYGFTVNTMKNSLVSYWNLGFTSVPQGQDLQYIFIPSDIRDYVMLRSAILGVPVPEGLDKGDRPHAYFGPEASASKFKLLHPDFISYLTERFLKSKLINTHFGDLYMPSTGALMLLTALHTCDQVSAYGFITSNYWKFSDHYFERKMKPLIFYANHDLSLEAALWRDLHKAGILQLYQR

SEQ ID NO: 46 - protein, 302 aa, Homo sapiens
MKAPGRLVLIILCSVVFSAVYILLCCWAGLPLCLATCLDHHFPTGSRPTVPGPLHFSGYSSVPDGKPLVREPCRSCAVVSSSGQMLGSGLGAEIDSAECVFRMNQAPTVGFEADVGQRSTLRVVSHTSVPLLLRNYSHYFQKARDTLYMVWGQGRHMDRVLGGRTYRTLLQLTRMYPGLQVYTFTERMMAYCDQIFQDETGKNRRQSGSFLSTGWFTMILALELCEEIVVYGMVSDSYCREKSHPSVPYHYFEKGRLDECQMYLAHEQAPRSAHRFITEKAVFSRWAKKRPIVFAHPSWRTE

SEQ ID NO: 47 - protein, 336 aa, Homo sapiens
MKTLMRHGLAVCLALTTMCTSLLLVYSSLGGQKERPPQQQQQQQQQQQQASATGSSQPAAESSTQQRPGVPAGPRPLDGYLGVADHKPLKMHCRDCALVTSSGHLLHSRQGSQIDQTECVIRMNDAPTRGYGRDVGNRTSLRVIAHSSIQRILRNRHDLLNVSQGTVFIFWGPSSYMRRDGKGQVYNNLHLLSQVLPRLKAFMITRHKMLQFDELFKQETGKDRKISNTWLSTGWFTMTIALELCDRINVYGMVPPDFCRDPNHPSVPYHYYEPFGPDECTMYLSHERGRKGSHHRFITEKRVFKNWARTFNIHFFQPDWKPESLAINHPENKPVF

SEQ ID NO: 48 - protein, 600 aa, Homo sapiens
MRSCLWRCRHLSQGVQWSLLLAVLVFFLFALPSFIKEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 49 - protein, 305 aa, Homo sapiens
MACILKRKSVIAVSFIAAFLFLLVVRLVNEVNFPLLLNCFGQPGTKWIPFSYTYRRPLRTHYGYINVKTQEPLQLDCDLCAIVSNSGQMVGQKVGNEIDRSSCIWRMNNAPTKGYEEDVGRMTMIRVVSHTSVPLLLKNPDYFFKEANTTIYVIWGPFRNMRKDGNGIVYNMLKKTVGIYPNAQIYVTTEKRMSYCDGVFKKETGKDRVQSGSYLSTGWFTFLLAMDACYGIHVYGMINDTYCKTEGYRKVPYHYYEQGRDECDEYFLHEHAPYGGHRFITEKKVFAKWAKKHRIIFTHPNWTLS

SEQ ID NO: 50 - protein, 333 aa, Homo sapiens
MACSRPPSQCEPTSLPPGPPAGRRHLPLSRRRREMSSNKEQRSAVFVILFALITILILYSSNSANEVFHYGSLRGRSRRPVNLKKWSITDGYVPILGNKTLPSRCHQCVIVSSSSHLLGTKLGPEIERAECTIRMNDAPTTGYSADVGNKTTYRVVAHSSVFRVLRRPQEFVNRTPETVFIFWGPPSKMQKPQGSLVRVIQRAGLVFPNMEAYAVSPGRMRQFDDLFRGETGKDREKSHSWLSTGWFTMVIAVELCDHVHVYGMVPPNYCSQRPRLQRMPYHYYEPKGPDECVTYIQNEHSRKGNHHRFITEKRVFSSWAQLYGITFSHPSWT

SEQ ID NO: 51 - protein, 529 aa, Homo sapiens
MKPHLKQWRQRMLFGIFAWGLLFLLIFIYFTDSNPAEPVPSSLSFLETRRLLPVQGKQRAIMGAAHEPSPPGGLDARQALPRAHPAGSFHAGPGDLQKWAQSQDGFEHKEFFSSQVGRKSQSAFYPEDDDYFFAAGQPGWHSHTQGTLGFPSPGEPGPREGAFPAAQVQRRRVKKRHRRQRRSHVLEEGDDGDRLYSSMSRAFLYRLWKGNVSSKMLNPRLQKAMKDYLTANKHGVRFRGKREAGLSRAQLLCQLRSRARVRTLDGTEAPFSALGWRRLVPAVPLSQLHPRGLRSCAVVMSAGAILNSSLGEEIDSHDAVLRFNSAPTRGYEKDVGNKTTIRIINSQILTNPSHHFIDSSLYKDVILVAWDPAPYSANLNLWYKKPDYNLFTPYIQHRQRNPNQPFYILHPKFIWQLWDIIQENTKEKIQPNPPSSGFIGILIMMSMCREVHVYEYIPSVRQTELCHYHELYYDAACTLGAYHPLLYEKLLVQRLNMGTQGDLHRKGKVVLPGFQAVHCPAPSPVIPHS

SEQ ID NO: 52 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 53 - protein, 377 aa, Neisseria meningitides
MKRILCITGTRADFGKLKPLLAYIENHPDLELHLIVTGMHMMKTYGRTYKEVTRENYQHTYLFSNQIQGEPMGAVLGNTITFISRLSDEIEPDMVMIHGDRLEALAGAAVGALSSRLVCHIEGGELSGTVDDSIRHSISKLSHIHLVANEQAVTRLVQMGEKRKHIHIIGSPDLDVMASSTLPSLEEVKEYYGLPYENYGISMFHPVTTEAHLMPQYAAQYFKALELSGQNIISIYPNNDTGTESILQELLKYQSDKFIAFPSIRFEYFLVLLKHAKFMVGNSSAGIREAPLYGVPSIDVGTRQSNRHMGKSIIHTDYETKNIFDAIQQACSLGKFEADDTFNGGDTRTSTERFAEVINNPETWNVSAQKRFIDLNL

SEQ ID NO: 54 - protein, 228 aa, Neisseria meningitides
MEKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILNHKES

SEQ ID NO: 55 - protein, 349 aa, Neisseria meningitides
MQNNNEFKIGNRSVGYNHEPLIICEIGINHEGSLKTAFEMVDAAYNAGAEVVKHQTHIVEDEMSDEAKQVIPGNADVSIYEIMERCALNEEDEIKLKEYVESKGMIFISTPFSRAAALRLQRMDIPAYKIGSGECNNYPLIKLVASFGKPIILSTGMNSIESIKKSVEIIREAGVPYALLHCTNIYPTPYEDVRLGGMNDLSEAFPDAIIGLSDHTLDNYACLGAVALGGSILERHFTDRMDRPGPDIVCSMNPDTFKELKQGAHALKLARGGKKDTIIAGEKPTKDFAFASVVADKDIKKGELLSGDNLWVKRPGNGDFSVNEYETLFGKVAACNIRKGAQIKKTDIE

SEQ ID NO: 56 - DNA, 10475 bp, Artificial Sequence
  Operon OGO-3
cggtacatgttcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcagatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagagttcggttccaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtagtctagaaggaggtattatgaaaattcttattagcggtggtgcaggttatataggttctcatactttaagacaatttttaaaaacagatcatgaaatttgtgttttagataatctttctaagggttctaaaatcgcaatagaagatttgcaaaaaacaagagcttttaaatttttcgaacaagatttaagtgattttcaaggcgtaaaagcattgtttgagagagaaaaatttgacgctattgtgcattttgcagcaagcattgaagtttttgaaagtatgcaaaatcctttaaaatattatatgaacaacactgttaatacgacaaatctcatcgaaacttgtttgcaaactggagtgaataaatttatattttcttcaacggcggccacttatggcgaaccacaaactcccgttgtgagcgaaacaagtcctttagcacctattaatccttatgggcgtagtaagcttatgagtgaagaagttttgcgtgatgcaagtatggcaaatcctgaatttaagcattgtattttaagatattttaatgttgcaggtgcttgtatggattatactttaggacaacgctatccaaaagcgactttgcttataaaagttgcagctgaatgtgccgcaggaaaacgtgataaacttttcatatttggcgatgattatgatacaaaagatggtacttgcataagagattttatccatgtagatgatatttcaagtgcacatttagcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagagcttatttgtaaatctgcttttgattgggaaaaacagtgttaataagcatgcggaaacagtcgacaggaggtattatggatgatgcggcgattcagcagaccctggccaaaatgggcattaaaagcagcgatattcagccggcaccggtggcgggtatgaaaaccgtgctgaccaacagcggcgtgctgtatatcaccgatgatggcaaacatatcattcagggcccgatgtatgatgtgagcggcaccgcaccggtgaacgtgaccaacaaaatgctgctgaaacagctgaacgcgctggaaaaagaaatgatcgtgtataaagcgccgcaggaaaaacacgttattaccgtgtttaccgatattacctgcggctattgccataaactgcatgagcaaatggcggattataacgcgctgggcattaccgtgcgttatctggcctttccgcgtcagggcctggatagcgatgccgaaaaggaaatgaaagcgatctggtgcgcgaaagataaaaacaaagcgttcgatgatgtgatggcgggcaaaagcgttgcgccggcgagctgtgatgtggatattgcggatcattatgcgctgggcgtgcagctgggtgttagcggcaccccggcggtggttctgagcaacggcaccctggtgccgggttatcagccgccgaaagagatgaaagaatttctggatgaacaccagaaaatgaccagcggcaaataatggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcacatatgcgcccatgcacatgcactcgctgtattgaagagcaacgcgtatccgcctggtttgatgaacgttttaatcgctccatgcagcctctgttaaccgctaaaaacgcccatttagaagaagatacctataagtggtggctccgcctgcaacgtgaaaaacaacccaacaaccttaacgacaccattcgcgaactgttccaggtagttcccggtaatgtggacccactgttggaaaaacgtcttgtttcatgtcgtcgttgcgccgtagttggcaactcaggtaaccttaaagaaagctattatggaccacaaattgattctcatgatttcgtattacgcatgaataaagcccccacagaaggtttcgaagctgacgttggcagtaaaacaacccaccattttgtataccctgaatcctttcgcgaacttgcccaagaagtttctatgatccttgtaccgtttaaaactactgatctcgaatgggtcatttcagcaactactaccggtcgcatttcccacacctacgttcctgtacctgccaaaattaaagtcaaaaaagaaaaaattttaatctaccacccagcatttattaaatacgtgtttgaccgttggcttcaaggtcacggccgttatccatccaccggcatcttaagcgtaattttttcactccacatctgcgatgaagtcgatctctacggtttcggggccgattcaaaaggaaactggcatcactattgggaaaataatccttcagctggcgcatttcgtaaaactggagttcatgatggagatttcgaaagcaatgtgaccaccattttagccagtattaataagattcgcatttttaaaggtcgctaagatccatcgatgcttaggaggtcatatgatgtttaaaatttcaatcatcttaccaacttataatgtggaacaatatatagcaagggcaatagaaagctgtatcaatcagacttttaaagatatagaaataattgtagttgatgattgtggaaatgataatagtataaatatagccaaagaatactctaaaaaagacaaaagaataaaaataatccacaatgaaaaaaacttaggtcttttaagagcaagatatgaaggtgtgaaagtagcaaactctccttatataatgtttttagatcctgatgattatttggaactaaatgcttgtgaagagtgtataaaaattttagatgaacaggatgaagttgatttagtgtttttcaatgctattgttgaaagtaatgttatttcatataaaaagtttgactttaattctggtttttatagcaaaaaagagtttgtaaaaaaaattattgcaaagaaaaatttatattggactatgtgggggaaacttataagaaagaaattgtatttagaagcttttgcgagtttaagactcgagaaagatgttaaaatcaatatggctgaagatgtattgttatattatccaatgttaagtcaagctcaaaaaatagcatatatgaactgtaatttatatcattacgtgcctaataataattcaatttgtaatactaagaatgaagtgcttgttaaaaataatattcaagagttgcagttggttttaaactatttaaggcaaaattatattttaaacaagtattgtagcgttctctatgtgctaattaaatatttgctatatattcaaatatataaaataaaaccgtgagaattccctgatcgatgataagctgtcaaacatgagcagatctgagcccgcctaatgagcgggcttttttttcagatctgcttgaagacgaaagggcctcgtgatacggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacc

SEQ ID NO: 57 - DNA, 9368 bp, Artificial Sequence
  Operon OGO-4
cggtacatgttcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcagatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagagttcggttccaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtagtctagaaggaggtattatgaaaattcttattagcggtggtgcaggttatataggttctcatactttaagacaatttttaaaaacagatcatgaaatttgtgttttagataatctttctaagggttctaaaatcgcaatagaagatttgcaaaaaacaagagcttttaaatttttcgaacaagatttaagtgattttcaaggcgtaaaagcattgtttgagagagaaaaatttgacgctattgtgcattttgcagcaagcattgaagtttttgaaagtatgcaaaatcctttaaaatattatatgaacaacactgttaatacgacaaatctcatcgaaacttgtttgcaaactggagtgaataaatttatattttcttcaacggcggccacttatggcgaaccacaaactcccgttgtgagcgaaacaagtcctttagcacctattaatccttatgggcgtagtaagcttatgagtgaagaagttttgcgtgatgcaagtatggcaaatcctgaatttaagcattgtattttaagatattttaatgttgcaggtgcttgtatggattatactttaggacaacgctatccaaaagcgactttgcttataaaagttgcagctgaatgtgccgcaggaaaacgtgataaacttttcatatttggcgatgattatgatacaaaagatggtacttgcataagagattttatccatgtagatgatatttcaagtgcacatttagcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagagcttatttgtaaatctgcttttgattgggaaaaacagtgttaataagcatgcggaaacagtcgacaggaggtattatggatgatgcggcgattcagcagaccctggccaaaatgggcattaaaagcagcgatattcagccggcaccggtggcgggtatgaaaaccgtgctgaccaacagcggcgtgctgtatatcaccgatgatggcaaacatatcattcagggcccgatgtatgatgtgagcggcaccgcaccggtgaacgtgaccaacaaaatgctgctgaaacagctgaacgcgctggaaaaagaaatgatcgtgtataaagcgccgcaggaaaaacacgttattaccgtgtttaccgatattacctgcggctattgccataaactgcatgagcaaatggcggattataacgcgctgggcattaccgtgcgttatctggcctttccgcgtcagggcctggatagcgatgccgaaaaggaaatgaaagcgatctggtgcgcgaaagataaaaacaaagcgttcgatgatgtgatggcgggcaaaagcgttgcgccggcgagctgtgatgtggatattgcggatcattatgcgctgggcgtgcagctgggtgttagcggcaccccggcggtggttctgagcaacggcaccctggtgccgggttatcagccgccgaaagagatgaaagaatttctggatgaacaccagaaaatgaccagcggcaaataatggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattatgtttaaaatttcaatcatcttaccaacttataatgtggaacaatatatagcaagggcaatagaaagctgtatcaatcagacttttaaagatatagaaataattgtagttgatgattgtggaaatgataatagtataaatatagccaaagaatactctaaaaaagacaaaagaataaaaataatccacaatgaaaaaaacttaggtcttttaagagcaagatatgaaggtgtgaaagtagcaaactctccttatataatgtttttagatcctgatgattatttggaactaaatgcttgtgaagagtgtataaaaattttagatgaacaggatgaagttgatttagtgtttttcaatgctattgttgaaagtaatgttatttcatataaaaagtttgactttaattctggtttttatagcaaaaaagagtttgtaaaaaaaattattgcaaagaaaaatttatattggactatgtgggggaaacttataagaaagaaattgtatttagaagcttttgcgagtttaagactcgagaaagatgttaaaatcaatatggctgaagatgtattgttatattatccaatgttaagtcaagctcaaaaaatagcatatatgaactgtaatttatatcattacgtgcctaataataattcaatttgtaatactaagaatgaagtgcttgttaaaaataatattcaagagttgcagttggttttaaactatttaaggcaaaattatattttaaacaagtattgtagcgttctctatgtgctaattaaatatttgctatatattcaaatatataaaataaaaccgtgagaattctctagaaataattttgtttaactttaagaaggagatataccatgacaaggactagaatggaaaatgaactcattgttagtaaaaatatgcaaaatataatcatagcaggaaatggacctagcctaaaaaatattaattataaaagactgcctagagaatatgatgtttttaggtgtaaccagttttattttgaagataagtattatttaggaaaaaagattaaagcagtattttttaatcctggtgtctttttacaacagtatcacactgcaaaacaacttatactaaaaaatgagtatgaaataaaaaatattttttgctctacatttaatttaccttttattgaaagcaatgattttttacatcaattttataattttttccccgatgcaaaacttggctatgaagttattgaaaaccttaaagaattttatgcttatataaaatacaatgaaatttatttcaataaaagaattacttcgggcgtctatatgtgtgcaattgctattgcattaggatataaaaccatctatttatgtggcattgatttttatgaaggagatgttatttatccttttgaagctatgagtacaaatataaaaacaatctttcctggaataaaagatttcaaaccttcaaattgtcattctaaggaatacgatatagaagcattaaaattgttaaaatcaatatacaaagttaatatctacgcattgtgtgatgattctattttggcaaatcattttcctttatcaattaatattaataacaatttcactttagaaaataagcataataattctataaatgatattttattgactgataatactcctggcgtaagtttttataaaaatcaacttaaagctgataataaaattatgcttaatttttataatattcttcattctaaagataatttaattaaatttttaaacaaataagtcgacctgcagaatgataagctgtcaaacatgagcagatctgagcccgcctaatgagcgggcttttttttcagatctgcttgaagacgaaagggcctcgtgatacggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacc

SEQ ID NO: 58 - DNA, 9308 bp, Artificial Sequence
  Operon OGO-5
cggtacatgttcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcagatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagagttcggttccaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtagtctagaaggaggtattatgaaaattcttattagcggtggtgcaggttatataggttctcatactttaagacaatttttaaaaacagatcatgaaatttgtgttttagataatctttctaagggttctaaaatcgcaatagaagatttgcaaaaaacaagagcttttaaatttttcgaacaagatttaagtgattttcaaggcgtaaaagcattgtttgagagagaaaaatttgacgctattgtgcattttgcagcaagcattgaagtttttgaaagtatgcaaaatcctttaaaatattatatgaacaacactgttaatacgacaaatctcatcgaaacttgtttgcaaactggagtgaataaatttatattttcttcaacggcggccacttatggcgaaccacaaactcccgttgtgagcgaaacaagtcctttagcacctattaatccttatgggcgtagtaagcttatgagtgaagaagttttgcgtgatgcaagtatggcaaatcctgaatttaagcattgtattttaagatattttaatgttgcaggtgcttgtatggattatactttaggacaacgctatccaaaagcgactttgcttataaaagttgcagctgaatgtgccgcaggaaaacgtgataaacttttcatatttggcgatgattatgatacaaaagatggtacttgcataagagattttatccatgtagatgatatttcaagtgcacatttagcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagagcttatttgtaaatctgcttttgattgggaaaaacagtgttaataagcatgcggaaacagtcgacaggaggtattatggatgatgcggcgattcagcagaccctggccaaaatgggcattaaaagcagcgatattcagccggcaccggtggcgggtatgaaaaccgtgctgaccaacagcggcgtgctgtatatcaccgatgatggcaaacatatcattcagggcccgatgtatgatgtgagcggcaccgcaccggtgaacgtgaccaacaaaatgctgctgaaacagctgaacgcgctggaaaaagaaatgatcgtgtataaagcgccgcaggaaaaacacgttattaccgtgtttaccgatattacctgcggctattgccataaactgcatgagcaaatggcggattataacgcgctgggcattaccgtgcgttatctggcctttccgcgtcagggcctggatagcgatgccgaaaaggaaatgaaagcgatctggtgcgcgaaagataaaaacaaagcgttcgatgatgtgatggcgggcaaaagcgttgcgccggcgagctgtgatgtggatattgcggatcattatgcgctgggcgtgcagctgggtgttagcggcaccccggcggtggttctgagcaacggcaccctggtgccgggttatcagccgccgaaagagatgaaagaatttctggatgaacaccagaaaatgaccagcggcaaataatggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattcatatgcgcccatgcacatgcactcgctgtattgaagagcaacgcgtatccgcctggtttgatgaacgttttaatcgctccatgcagcctctgttaaccgctaaaaacgcccatttagaagaagatacctataagtggtggctccgcctgcaacgtgaaaaacaacccaacaaccttaacgacaccattcgcgaactgttccaggtagttcccggtaatgtggacccactgttggaaaaacgtcttgtttcatgtcgtcgttgcgccgtagttggcaactcaggtaaccttaaagaaagctattatggaccacaaattgattctcatgatttcgtattacgcatgaataaagcccccacagaaggtttcgaagctgacgttggcagtaaaacaacccaccattttgtataccctgaatcctttcgcgaacttgcccaagaagtttctatgatccttgtaccgtttaaaactactgatctcgaatgggtcatttcagcaactactaccggtcgcatttcccacacctacgttcctgtacctgccaaaattaaagtcaaaaaagaaaaaattttaatctaccacccagcatttattaaatacgtgtttgaccgttggcttcaaggtcacggccgttatccatccaccggcatcttaagcgtaattttttcactccacatctgcgatgaagtcgatctctacggtttcggggccgattcaaaaggaaactggcatcactattgggaaaataatccttcagctggcgcatttcgtaaaactggagttcatgatggagatttcgaaagcaatgtgaccaccattttagccagtattaataagattcgcatttttaaaggtcgctaagatccatcgatgcttaggaggtcatatgatgtttaaaatttcaatcatcttaccaacttataatgtggaacaatatatagcaagggcaatagaaagctgtatcaatcagacttttaaagatatagaaataattgtagttgatgattgtggaaatgataatagtataaatatagccaaagaatactctaaaaaagacaaaagaataaaaataatccacaatgaaaaaaacttaggtcttttaagagcaagatatgaaggtgtgaaagtagcaaactctccttatataatgtttttagatcctgatgattatttggaactaaatgcttgtgaagagtgtataaaaattttagatgaacaggatgaagttgatttagtgtttttcaatgctattgttgaaagtaatgttatttcatataaaaagtttgactttaattctggtttttatagcaaaaaagagtttgtaaaaaaaattattgcaaagaaaaatttatattggactatgtgggggaaacttataagaaagaaattgtatttagaagcttttgcgagtttaagactcgagaaagatgttaaaatcaatatggctgaagatgtattgttatattatccaatgttaagtcaagctcaaaaaatagcatatatgaactgtaatttatatcattacgtgcctaataataattcaatttgtaatactaagaatgaagtgcttgttaaaaataatattcaagagttgcagttggttttaaactatttaaggcaaaattatattttaaacaagtattgtagcgttctctatgtgctaattaaatatttgctatatattcaaatatataaaataaaaccgtgagaattccctgatcgatgataagctgtcaaacatgagcagatctgagcccgcctaatgagcgggcttttttttcagatctgcttgaagacgaaagggcctcgtgatacggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacc

SEQ ID NO: 59 - DNA, 9320 bp, Artificial Sequence
  Operon OGO-6
cggtacatgttcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcagatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagagttcggttccaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtagtctagaaggaggtattatgaaaattcttattagcggtggtgcaggttatataggttctcatactttaagacaatttttaaaaacagatcatgaaatttgtgttttagataatctttctaagggttctaaaatcgcaatagaagatttgcaaaaaacaagagcttttaaatttttcgaacaagatttaagtgattttcaaggcgtaaaagcattgtttgagagagaaaaatttgacgctattgtgcattttgcagcaagcattgaagtttttgaaagtatgcaaaatcctttaaaatattatatgaacaacactgttaatacgacaaatctcatcgaaacttgtttgcaaactggagtgaataaatttatattttcttcaacggcggccacttatggcgaaccacaaactcccgttgtgagcgaaacaagtcctttagcacctattaatccttatgggcgtagtaagcttatgagtgaagaagttttgcgtgatgcaagtatggcaaatcctgaatttaagcattgtattttaagatattttaatgttgcaggtgcttgtatggattatactttaggacaacgctatccaaaagcgactttgcttataaaagttgcagctgaatgtgccgcaggaaaacgtgataaacttttcatatttggcgatgattatgatacaaaagatggtacttgcataagagattttatccatgtagatgatatttcaagtgcacatttagcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagagcttatttgtaaatctgcttttgattgggaaaaacagtgttaataagcatgcggaaacagtcgacaggaggtattatggatgatgcggcgattcagcagaccctggccaaaatgggcattaaaagcagcgatattcagccggcaccggtggcgggtatgaaaaccgtgctgaccaacagcggcgtgctgtatatcaccgatgatggcaaacatatcattcagggcccgatgtatgatgtgagcggcaccgcaccggtgaacgtgaccaacaaaatgctgctgaaacagctgaacgcgctggaaaaagaaatgatcgtgtataaagcgccgcaggaaaaacacgttattaccgtgtttaccgatattacctgcggctattgccataaactgcatgagcaaatggcggattataacgcgctgggcattaccgtgcgttatctggcctttccgcgtcagggcctggatagcgatgccgaaaaggaaatgaaagcgatctggtgcgcgaaagataaaaacaaagcgttcgatgatgtgatggcgggcaaaagcgttgcgccggcgagctgtgatgtggatattgcggatcattatgcgctgggcgtgcagctgggtgttagcggcaccccggcggtggttctgagcaacggcaccctggtgccgggttatcagccgccgaaagagatgaaagaatttctggatgaacaccagaaaatgaccagcggcaaataatggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattatgtttaaaatttcaatcatcttaccaacttataatgtggaacaatatatagcaagggcaatagaaagctgtatcaatcagacttttaaagatatagaaataattgtagttgatgattgtggaaatgataatagtataaatatagccaaagaatactctaaaaaagacaaaagaataaaaataatccacaatgaaaaaaacttaggtcttttaagagcaagatatgaaggtgtgaaagtagcaaactctccttatataatgtttttagatcctgatgattatttggaactaaatgcttgtgaagagtgtataaaaattttagatgaacaggatgaagttgatttagtgtttttcaatgctattgttgaaagtaatgttatttcatataaaaagtttgactttaattctggtttttatagcaaaaaagagtttgtaaaaaaaattattgcaaagaaaaatttatattggactatgtgggggaaacttataagaaagaaattgtatttagaagcttttgcgagtttaagactcgagaaagatgttaaaatcaatatggctgaagatgtattgttatattatccaatgttaagtcaagctcaaaaaatagcatatatgaactgtaatttatatcattacgtgcctaataataattcaatttgtaatactaagaatgaagtgcttgttaaaaataatattcaagagttgcagttggttttaaactatttaaggcaaaattatattttaaacaagtattgtagcgttctctatgtgctaattaaatatttgctatatattcaaatatataaaataaaaccgtgagaattctctagaaataattttgtttaactttaagaaggagatataccatgcgcccatgcacatgcactcgctgtattgaagagcaacgcgtatccgcctggtttgatgaacgttttaatcgctccatgcagcctctgttaaccgctaaaaacgcccatttagaagaagatacctataagtggtggctccgcctgcaacgtgaaaaacaacccaacaaccttaacgacaccattcgcgaactgttccaggtagttcccggtaatgtggacccactgttggaaaaacgtcttgtttcatgtcgtcgttgcgccgtagttggcaactcaggtaaccttaaagaaagctattatggaccacaaattgattctcatgatttcgtattacgcatgaataaagcccccacagaaggtttcgaagctgacgttggcagtaaaacaacccaccattttgtataccctgaatcctttcgcgaacttgcccaagaagtttctatgatccttgtaccgtttaaaactactgatctcgaatgggtcatttcagcaactactaccggtcgcatttcccacacctacgttcctgtacctgccaaaattaaagtcaaaaaagaaaaaattttaatctaccacccagcatttattaaatacgtgtttgaccgttggcttcaaggtcacggccgttatccatccaccggcatcttaagcgtaattttttcactccacatctgcgatgaagtcgatctctacggtttcggggccgattcaaaaggaaactggcatcactattgggaaaataatccttcagctggcgcatttcgtaaaactggagttcatgatggagatttcgaaagcaatgtgaccaccattttagccagtattaataagattcgcatttttaaaggtcgctaagatccatcgattgataagctgtcaaacatgagcagatctgagcccgcctaatgagcgggcttttttttcagatctgcttgaagacgaaagggcctcgtgatacggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacc

SEQ ID NO: 60 - DNA, 10147 bp, Artificial Sequence
  Operon OGO-7
cggtacatgttcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcagatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagagttcggttccaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtagtctagaaggaggtattatgaaaattcttattagcggtggtgcaggttatataggttctcatactttaagacaatttttaaaaacagatcatgaaatttgtgttttagataatctttctaagggttctaaaatcgcaatagaagatttgcaaaaaacaagagcttttaaatttttcgaacaagatttaagtgattttcaaggcgtaaaagcattgtttgagagagaaaaatttgacgctattgtgcattttgcagcaagcattgaagtttttgaaagtatgcaaaatcctttaaaatattatatgaacaacactgttaatacgacaaatctcatcgaaacttgtttgcaaactggagtgaataaatttatattttcttcaacggcggccacttatggcgaaccacaaactcccgttgtgagcgaaacaagtcctttagcacctattaatccttatgggcgtagtaagcttatgagtgaagaagttttgcgtgatgcaagtatggcaaatcctgaatttaagcattgtattttaagatattttaatgttgcaggtgcttgtatggattatactttaggacaacgctatccaaaagcgactttgcttataaaagttgcagctgaatgtgccgcaggaaaacgtgataaacttttcatatttggcgatgattatgatacaaaagatggtacttgcataagagattttatccatgtagatgatatttcaagtgcacatttagcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagagcttatttgtaaatctgcttttgattgggaaaaacagtgttaataagcatgcggaaacagtcgacaggaggtattatggatgatgcggcgattcagcagaccctggccaaaatgggcattaaaagcagcgatattcagccggcaccggtggcgggtatgaaaaccgtgctgaccaacagcggcgtgctgtatatcaccgatgatggcaaacatatcattcagggcccgatgtatgatgtgagcggcaccgcaccggtgaacgtgaccaacaaaatgctgctgaaacagctgaacgcgctggaaaaagaaatgatcgtgtataaagcgccgcaggaaaaacacgttattaccgtgtttaccgatattacctgcggctattgccataaactgcatgagcaaatggcggattataacgcgctgggcattaccgtgcgttatctggcctttccgcgtcagggcctggatagcgatgccgaaaaggaaatgaaagcgatctggtgcgcgaaagataaaaacaaagcgttcgatgatgtgatggcgggcaaaagcgttgcgccggcgagctgtgatgtggatattgcggatcattatgcgctgggcgtgcagctgggtgttagcggcaccccggcggtggttctgagcaacggcaccctggtgccgggttatcagccgccgaaagagatgaaagaatttctggatgaacaccagaaaatgaccagcggcaaataatggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattcatatgcgcccatgcacatgcactcgctgtattgaagagcaacgcgtatccgcctggtttgatgaacgttttaatcgctccatgcagcctctgttaaccgctaaaaacgcccatttagaagaagatacctataagtggtggctccgcctgcaacgtgaaaaacaacccaacaaccttaacgacaccattcgcgaactgttccaggtagttcccggtaatgtggacccactgttggaaaaacgtcttgtttcatgtcgtcgttgcgccgtagttggcaactcaggtaaccttaaagaaagctattatggaccacaaattgattctcatgatttcgtattacgcatgaataaagcccccacagaaggtttcgaagctgacgttggcagtaaaacaacccaccattttgtataccctgaatcctttcgcgaacttgcccaagaagtttctatgatccttgtaccgtttaaaactactgatctcgaatgggtcatttcagcaactactaccggtcgcatttcccacacctacgttcctgtacctgccaaaattaaagtcaaaaaagaaaaaattttaatctaccacccagcatttattaaatacgtgtttgaccgttggcttcaaggtcacggccgttatccatccaccggcatcttaagcgtaattttttcactccacatctgcgatgaagtcgatctctacggtttcggggccgattcaaaaggaaactggcatcactattgggaaaataatccttcagctggcgcatttcgtaaaactggagttcatgatggagatttcgaaagcaatgtgaccaccattttagccagtattaataagattcgcatttttaaaggtcgctaagatccatcgatgcttaggaggtcatatgatgtttaaaatttcaatcatcttaccaacttataatgtggaacaatatatagcaagggcaatagaaagctgtatcaatcagacttttaaagatatagaaataattgtagttgatgattgtggaaatgataatagtataaatatagccaaagaatactctaaaaaagacaaaagaataaaaataatccacaatgaaaaaaacttaggtcttttaagagcaagatatgaaggtgtgaaagtagcaaactctccttatataatgtttttagatcctgatgattatttggaactaaatgcttgtgaagagtgtataaaaattttagatgaacaggatgaagttgatttagtgtttttcaatgctattgttgaaagtaatgttatttcatataaaaagtttgactttaattctggtttttatagcaaaaaagagtttgtaaaaaaaattattgcaaagaaaaatttatattggactatgtgggggaaacttataagaaagaaattgtatttagaagcttttgcgagtttaagactcgagaaagatgttaaaatcaatatggctgaagatgtattgttatattatccaatgttaagtcaagctcaaaaaatagcatatatgaactgtaatttatatcattacgtgcctaataataattcaatttgtaatactaagaatgaagtgcttgttaaaaataatattcaagagttgcagttggttttaaactatttaaggcaaaattatattttaaacaagtattgtagcgttctctatgtgctaattaaatatttgctatatattcaaatatataaaataaaaccgtgagaattccctggtataagaaggagatatacatatgacctgcctggatcaccacttcccgaccggttctcgtccgaccgttccgggtccgctgcacttcagcggttatagtagcgttccggatggtaaaccgctggttcgtgaaccgtgtcgtagctgcgctgtggtttcttcttctggccagatgctgggtagtggtctgggtgcggaaattgattctgctgaatgtgttttccgtatgaaccaagccccgactgttggttttgaagctgatgttggtcagcgttctaccctgcgtgttgtttcccacacctctgttccgctgctgctgcgtaactatagccattatttccaaaaagcgcgtgataccctgtatatggtttggggtcagggtcgtcacatggatcgtgttctgggtggtcgtacctatcgtaccctgctgcagctgacccgtatgtatccgggtctgcaggtttacaccttcaccgaacgtatgatggcatactgtgatcagatctttcaggatgaaactggcaaaaaccgtcgtcagagcggttctttcctgtctactggttggtttacgatgatcctggctctggaactgtgtgaagaaatcgttgtttatggtatggtttccgatagctattgccgtgaaaaaagccacccgtctgttccgtatcattactttgaaaaaggtcgtctggatgaatgccagatgtacctggcacatgaacaggctccgcgttctgctcatcgttttatcactgaaaaagcagttttctctcgttgggctaaaaaacgtccgattgtttttgcacacccgtcttggcgtaccgaataagtcgagtctggatcgatgataagctgtcaaacatgagcagatctgagcccgcctaatgagcgggcttttttttcagatctgcttgaagacgaaagggcctcgtgatacggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacc

SEQ ID NO: 61 - DNA, 10158 bp, Artificial Sequence
  Operon OGO-8
cggtacatgttcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcagatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagagttcggttccaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtagtctaggtataagaaggagatatacatatgacctgcctggatcaccacttcccgaccggttctcgtccgaccgttccgggtccgctgcacttcagcggttatagtagcgttccggatggtaaaccgctggttcgtgaaccgtgtcgtagctgcgctgtggtttcttcttctggccagatgctgggtagtggtctgggtgcggaaattgattctgctgaatgtgttttccgtatgaaccaagccccgactgttggttttgaagctgatgttggtcagcgttctaccctgcgtgttgtttcccacacctctgttccgctgctgctgcgtaactatagccattatttccaaaaagcgcgtgataccctgtatatggtttggggtcagggtcgtcacatggatcgtgttctgggtggtcgtacctatcgtaccctgctgcagctgacccgtatgtatccgggtctgcaggtttacaccttcaccgaacgtatgatggcatactgtgatcagatctttcaggatgaaactggcaaaaaccgtcgtcagagcggttctttcctgtctactggttggtttacgatgatcctggctctggaactgtgtgaagaaatcgttgtttatggtatggtttccgatagctattgccgtgaaaaaagccacccgtctgttccgtatcattactttgaaaaaggtcgtctggatgaatgccagatgtacctggcacatgaacaggctccgcgttctgctcatcgttttatcactgaaaaagcagttttctctcgttgggctaaaaaacgtccgattgtttttgcacacccgtcttggcgtaccgaataagtcgagtctggtaaagaaaccgaaggaggtattatgaaaattcttattagcggtggtgcaggttatataggttctcatactttaagacaatttttaaaaacagatcatgaaatttgtgttttagataatctttctaagggttctaaaatcgcaatagaagatttgcaaaaaacaagagcttttaaatttttcgaacaagatttaagtgattttcaaggcgtaaaagcattgtttgagagagaaaaatttgacgctattgtgcattttgcagcaagcattgaagtttttgaaagtatgcaaaatcctttaaaatattatatgaacaacactgttaatacgacaaatctcatcgaaacttgtttgcaaactggagtgaataaatttatattttcttcaacggcggccacttatggcgaaccacaaactcccgttgtgagcgaaacaagtcctttagcacctattaatccttatgggcgtagtaagcttatgagtgaagaagttttgcgtgatgcaagtatggcaaatcctgaatttaagcattgtattttaagatattttaatgttgcaggtgcttgtatggattatactttaggacaacgctatccaaaagcgactttgcttataaaagttgcagctgaatgtgccgcaggaaaacgtgataaacttttcatatttggcgatgattatgatacaaaagatggtacttgcataagagattttatccatgtagatgatatttcaagtgcacatttagcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagagcttatttgtaaatctgcttttgattgggaaaaacagtgttaataagcatgcggaaacagtcgacaggaggtattatggatgatgcggcgattcagcagaccctggccaaaatgggcattaaaagcagcgatattcagccggcaccggtggcgggtatgaaaaccgtgctgaccaacagcggcgtgctgtatatcaccgatgatggcaaacatatcattcagggcccgatgtatgatgtgagcggcaccgcaccggtgaacgtgaccaacaaaatgctgctgaaacagctgaacgcgctggaaaaagaaatgatcgtgtataaagcgccgcaggaaaaacacgttattaccgtgtttaccgatattacctgcggctattgccataaactgcatgagcaaatggcggattataacgcgctgggcattaccgtgcgttatctggcctttccgcgtcagggcctggatagcgatgccgaaaaggaaatgaaagcgatctggtgcgcgaaagataaaaacaaagcgttcgatgatgtgatggcgggcaaaagcgttgcgccggcgagctgtgatgtggatattgcggatcattatgcgctgggcgtgcagctgggtgttagcggcaccccggcggtggttctgagcaacggcaccctggtgccgggttatcagccgccgaaagagatgaaagaatttctggatgaacaccagaaaatgaccagcggcaaataatggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattcatatgcgcccatgcacatgcactcgctgtattgaagagcaacgcgtatccgcctggtttgatgaacgttttaatcgctccatgcagcctctgttaaccgctaaaaacgcccatttagaagaagatacctataagtggtggctccgcctgcaacgtgaaaaacaacccaacaaccttaacgacaccattcgcgaactgttccaggtagttcccggtaatgtggacccactgttggaaaaacgtcttgtttcatgtcgtcgttgcgccgtagttggcaactcaggtaaccttaaagaaagctattatggaccacaaattgattctcatgatttcgtattacgcatgaataaagcccccacagaaggtttcgaagctgacgttggcagtaaaacaacccaccattttgtataccctgaatcctttcgcgaacttgcccaagaagtttctatgatccttgtaccgtttaaaactactgatctcgaatgggtcatttcagcaactactaccggtcgcatttcccacacctacgttcctgtacctgccaaaattaaagtcaaaaaagaaaaaattttaatctaccacccagcatttattaaatacgtgtttgaccgttggcttcaaggtcacggccgttatccatccaccggcatcttaagcgtaattttttcactccacatctgcgatgaagtcgatctctacggtttcggggccgattcaaaaggaaactggcatcactattgggaaaataatccttcagctggcgcatttcgtaaaactggagttcatgatggagatttcgaaagcaatgtgaccaccattttagccagtattaataagattcgcatttttaaaggtcgctaagatccatcgatgcttaggaggtcatatgatgtttaaaatttcaatcatcttaccaacttataatgtggaacaatatatagcaagggcaatagaaagctgtatcaatcagacttttaaagatatagaaataattgtagttgatgattgtggaaatgataatagtataaatatagccaaagaatactctaaaaaagacaaaagaataaaaataatccacaatgaaaaaaacttaggtcttttaagagcaagatatgaaggtgtgaaagtagcaaactctccttatataatgtttttagatcctgatgattatttggaactaaatgcttgtgaagagtgtataaaaattttagatgaacaggatgaagttgatttagtgtttttcaatgctattgttgaaagtaatgttatttcatataaaaagtttgactttaattctggtttttatagcaaaaaagagtttgtaaaaaaaattattgcaaagaaaaatttatattggactatgtgggggaaacttataagaaagaaattgtatttagaagcttttgcgagtttaagactcgagaaagatgttaaaatcaatatggctgaagatgtattgttatattatccaatgttaagtcaagctcaaaaaatagcatatatgaactgtaatttatatcattacgtgcctaataataattcaatttgtaatactaagaatgaagtgcttgttaaaaataatattcaagagttgcagttggttttaaactatttaaggcaaaattatattttaaacaagtattgtagcgttctctatgtgctaattaaatatttgctatatattcaaatatataaaataaaaccgtgagaattccctgatcgatgataagctgtcaaacatgagcagatctgagcccgcctaatgagcgggcttttttttcagatctgcttgaagacgaaagggcctcgtgatacggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacc

SEQ ID NO: 62 - DNA, 10324 bp, Artificial Sequence
  Operon OGO-9
cggtacatgttcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcagatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagagttcggttccaaaaagaaagaccttcatcacagcaatggagaagagaaagcacaaagcatggagaccctccctccagggaaagtacggtggccagactttaaccaggaagcttatgttggagggacgatggtccgctccgggcaggacccttacgcccgcaacaagttcaaccaggtggagagtgataagcttcgaatggacagagccatccctgacacccggcatgaccagtgtcagcggaagcagtggcgggtggatctgccggccaccagcgtggtgatcacgtttcacaatgaagccaggtcggccctactcaggaccgtggtcagcgtgcttaagaaaagcccgccccatctcataaaagaaatcatcttggtggatgactacagcaatgatcctgaggacggggctctcttggggaaaattgagaaagtgcgagttcttagaaatgatcgacgagaaggcctcatgcgctcacgggttcggggggccgatgctgcccaagccaaggtcctgaccttcctggacagtcactgcgagtgtaatgagcactggctggagcccctcctggaaagggtggcggaggacaggactcgggttgtgtcacccatcatcgatgtcattaatatggacaactttcagtatgtgggggcatctgctgacttgaagggcggttttgattggaacttggtattcaagtgggattacatgacgcctgagcagagaaggtcccggcaggggaacccagtcgcccctataaaaacccccatgattgctggtgggctgtttgtgatggataagttctattttgaagaactggggaagtacgacatgatgatggatgtgtggggaggagagaacctagagatctcgttccgcgtgtggcagtgtggtggcagcctggagatcatcccgtgcagccgtgtgggacacgtgttccggaagcagcacccctacacgttcccgggtggcagtggcactgtctttgcccgaaacacccgccgggcagcagaggtctggatggatgaatacaaaaatttctattatgcagcagtgccttctgctagaaacgttccttatggaaatattcagagcagattggagcttaggaagaaactcagctgcaagcctttcaaatggtaccttgaaaatgtctatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtagtctaggtataagaaggagatatacatatgatgtctaaggcatccaacagctggacgggtaagggtcaagcatgtcgtcacctgctgcatctggctatccaacgtcaccctcacttccgtggtctgttcaacctgagcatcccagttctgctgtggggtgatctgttcacgcctgcactgtgggatcgtctgtctcagcataaagcaccgtatggttggcgtggtctgtctcatcaggtaatcgcatctaccctgtctctgctgaacggtagcgaaagcgctaagctgttcgctccaccacgtgataccccgccgaaatgtatccgttgcgctgtagtaggtaacggtggtatcctgaacggttctcgtcagggtccgaatatcgatgctcacgattacgtcttccgtctgaatggcgctgtcatcaagggcttcgagcgtgatgtgggtaccaaaacctccttctacggcttcaccgtgaacaccatgaaaaacagcctggtgtcctactggaacctgggcttcacctccgtgccgcagggtcaggatctgcagtatatcttcatcccgtctgacatccgtgactacgttatgctgcgctccgccatcctgggcgttccggttccggaaggcctggataaaggcgaccgcccgcatgcgtattttggcccggaagcctctgcgtctaaatttaaactgctgcacccggactttatttcttatctgactgaacgcttcctgaaatccaaactgattaacacccacttcggcgacctgtacatgccgtccactggcgcgctgatgctgctgactgccctgcacacttgcgaccaggttagcgcctatggctttattactagcaactactggaaattcagcgaccactactttgagcgcaaaatgaaaccgctgattttttacgcgaaccacgacctgtccctggaagcggcgctgtggcgcgacctgcacaaagcgggcattctgcagctgtaccagcgctaagtcgagtctggtaaagaaacaaggaggtattatgaaaattcttattagcggtggtgcaggttatataggttctcatactttaagacaatttttaaaaacagatcatgaaatttgtgttttagataatctttctaagggttctaaaatcgcaatagaagatttgcaaaaaacaagagcttttaaatttttcgaacaagatttaagtgattttcaaggcgtaaaagcattgtttgagagagaaaaatttgacgctattgtgcattttgcagcaagcattgaagtttttgaaagtatgcaaaatcctttaaaatattatatgaacaacactgttaatacgacaaatctcatcgaaacttgtttgcaaactggagtgaataaatttatattttcttcaacggcggccacttatggcgaaccacaaactcccgttgtgagcgaaacaagtcctttagcacctattaatccttatgggcgtagtaagcttatgagtgaagaagttttgcgtgatgcaagtatggcaaatcctgaatttaagcattgtattttaagatattttaatgttgcaggtgcttgtatggattatactttaggacaacgctatccaaaagcgactttgcttataaaagttgcagctgaatgtgccgcaggaaaacgtgataaacttttcatatttggcgatgattatgatacaaaagatggtacttgcataagagattttatccatgtagatgatatttcaagtgcacatttagcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagagcttatttgtaaatctgcttttgattgggaaaaacagtgttaataagcatgcggaaacagtcgacaggaggtattatggatgatgcggcgattcagcagaccctggccaaaatgggcattaaaagcagcgatattcagccggcaccggtggcgggtatgaaaaccgtgctgaccaacagcggcgtgctgtatatcaccgatgatggcaaacatatcattcagggcccgatgtatgatgtgagcggcaccgcaccggtgaacgtgaccaacaaaatgctgctgaaacagctgaacgcgctggaaaaagaaatgatcgtgtataaagcgccgcaggaaaaacacgttattaccgtgtttaccgatattacctgcggctattgccataaactgcatgagcaaatggcggattataacgcgctgggcattaccgtgcgttatctggcctttccgcgtcagggcctggatagcgatgccgaaaaggaaatgaaagcgatctggtgcgcgaaagataaaaacaaagcgttcgatgatgtgatggcgggcaaaagcgttgcgccggcgagctgtgatgtggatattgcggatcattatgcgctgggcgtgcagctgggtgttagcggcaccccggcggtggttctgagcaacggcaccctggtgccgggttatcagccgccgaaagagatgaaagaatttctggatgaacaccagaaaatgaccagcggcaaataatggcaaatattctgaaatgagctgttgacaattaatcatcggctcgtataatgtgtggaattgtgagcggataacaatttcacacaggaaacagccagtccgtttaggtgttttcacgagcaattgaccaacaaggaccatagattcatatgcgcccatgcacatgcactcgctgtattgaagagcaacgcgtatccgcctggtttgatgaacgttttaatcgctccatgcagcctctgttaaccgctaaaaacgcccatttagaagaagatacctataagtggtggctccgcctgcaacgtgaaaaacaacccaacaaccttaacgacaccattcgcgaactgttccaggtagttcccggtaatgtggacccactgttggaaaaacgtcttgtttcatgtcgtcgttgcgccgtagttggcaactcaggtaaccttaaagaaagctattatggaccacaaattgattctcatgatttcgtattacgcatgaataaagcccccacagaaggtttcgaagctgacgttggcagtaaaacaacccaccattttgtataccctgaatcctttcgcgaacttgcccaagaagtttctatgatccttgtaccgtttaaaactactgatctcgaatgggtcatttcagcaactactaccggtcgcatttcccacacctacgttcctgtacctgccaaaattaaagtcaaaaaagaaaaaattttaatctaccacccagcatttattaaatacgtgtttgaccgttggcttcaaggtcacggccgttatccatccaccggcatcttaagcgtaattttttcactccacatctgcgatgaagtcgatctctacggtttcggggccgattcaaaaggaaactggcatcactattgggaaaataatccttcagctggcgcatttcgtaaaactggagttcatgatggagatttcgaaagcaatgtgaccaccattttagccagtattaataagattcgcatttttaaaggtcgctaagatccatcgatgcttaggaggtcatatgatgtttaaaatttcaatcatcttaccaacttataatgtggaacaatatatagcaagggcaatagaaagctgtatcaatcagacttttaaagatatagaaataattgtagttgatgattgtggaaatgataatagtataaatatagccaaagaatactctaaaaaagacaaaagaataaaaataatccacaatgaaaaaaacttaggtcttttaagagcaagatatgaaggtgtgaaagtagcaaactctccttatataatgtttttagatcctgatgattatttggaactaaatgcttgtgaagagtgtataaaaattttagatgaacaggatgaagttgatttagtgtttttcaatgctattgttgaaagtaatgttatttcatataaaaagtttgactttaattctggtttttatagcaaaaaagagtttgtaaaaaaaattattgcaaagaaaaatttatattggactatgtgggggaaacttataagaaagaaattgtatttagaagcttttgcgagtttaagactcgagaaagatgttaaaatcaatatggctgaagatgtattgttatattatccaatgttaagtcaagctcaaaaaatagcatatatgaactgtaatttatatcattacgtgcctaataataattcaatttgtaatactaagaatgaagtgcttgttaaaaataatattcaagagttgcagttggttttaaactatttaaggcaaaattatattttaaacaagtattgtagcgttctctatgtgctaattaaatatttgctatatattcaaatatataaaataaaaccgtgagaattccctgatcgatgataagctgtcaaacatgagcagatctgagcccgcctaatgagcgggcttttttttcagatctgcttgaagacgaaagggcctcgtgatacggagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatctggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacc

SEQ ID NO: 63 - DNA, 3332 bp, Neisseria meningitides
  misc_feature (1058)..(1058): n is a, c, g, or t
  misc_feature (1991)..(1991): n is a, c, g, or t
  misc_feature (2025)..(2025): n is a, c, g, or t
cacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggtcatatgaaaagaattctttgcattacaggtaccagagccgacttcggcaagctaaaacctttattagcctatattgaaaatcacccagaccttgaattgcatttgattgtaactggtatgcacatgatgaaaacctatggcagaacctacaaggaagtaactcgagaaaactatcaacatacatatctgttttcaaatcaaatccaaggtgaaccaatgggtgccgttttaggcaataccattacgtttatctctcgtctatctgatgaaattgaacctgatatggtcatgattcacggcgaccgtttagaagcactagcaggcgcagctgtaggtgcattaagcagccgtttagtttgccatatcgaaggtggtgaactatctggtacagtagatgactccattcgtcattctattagtaaactttctcatatccacttggtagcaaatgaacaagctgtcactcgcctagtgcaaatgggagaaaaaagaaagcatattcacatcatcggctcccccgatttagatgttatggcctcttccaccctcccatccttagaagaagtcaaagaatattacggtttaccatacgaaaattatggtatttctatgtttcaccccgtgactacagaagcacatttaatgccacaatatgcggcccaatatttcaaagcattanaattaagtggccaaaatatcattagcatctaccctaataatgacactggcactgaaagtattctgcaagaattattaaaatatcaatctgataaatttattgcatttccttccattcgatttgaatattttttagtcttattgaaacatgctaaatttatggtcggcaactcaagtgcaggtattagagaagctcctctctacggtgtcccttcaattgatgttggtacacgccaaagtaaccgccacatgggaaaatctattattcatacagattatgaaactaaaaatatctttgatgcgattcaacaagcatgtagtttaggcaaatttgaagcagatgatacctttaatggcggagatactcgcaccagcacagaaagatttgctgaagtaatcaacaatcctgaaacgtggaatgtttctgctcaaaaacgttttatcgatttgaatctttaaattatggaaaaacaaaatattgcggttatacttgcgcgccaaaactccaaaggattgccattaaaaaatctccggaaaatgaatggcatatcattacttggtcatacaattaatgctgctatatcatcaaagtgttttgaccgcataattgtttcgactgatggcgggttaattgcagaagaagctaaaaatttcggtgtcgaagtcgtcctacgccctgcagagctggcctccgatacagccagctctatttcaggtgtaatacatgctttagaaacaattggcagtaattccggcacagtaaccctattacaaccaaccagtccattacgcacaggggctcatattcgtgaagctttttctctatttgatgagaaaataaaaggaagcgttgtctctgcatgtccaatggagcatcatccactaaaaaccctgcttcaaatcaataatggcgaatatgcccccatgcgccatntaagcgatttggagcagcctcgccaacaattacntcaggcatttaggcctaatggtgcaatttacattaatgatactgcttcactaattgcaaataattgtttttttatcgccccaaccaaactttatattatgtctcatcaagactctatcgatattgatactgagcttgatttacaacaggcagaaaacattcttaatcacaaggaaagctaaatgcaaaacaacaacgaatttaaaattggtaatcgttcagtaggttacaaccacgaaccattgattatctgtgaaatcggcatcaatcatgaaggctctttaaaaacagcttttgaaatggttgatgctgcctataatgcaggcgctgaagttgttaaacatcaaacacacatcgttgaagacgaaatgtctgatgaggccaaacaagtcattccaggcaatgcagatgtctctatttatgaaattatggaacgttgcgccctgaatgaagaagatgagattaaattaaaagaatacgtagagagtaagggtatgatttttatcagtactcctttctctcgtgcagctgctttacgattacaacgtatggatattccagcatataaaatcggctctggcgaatgtaataactacccattaattaaactggtggcctcttttggtaagcctattattctctctaccggcatgaattctattgaaagcatcaaaaagtcggtagaaattattcgagaagcaggggtaccttatgctttgcttcactgtaccaacatctacccaaccccttacgaagatgttcgattgggtggtatgaacgatttatctgaagcctttccagacgcaatcattggcctgtctgaccataccttagataactatgcttgcttaggagcagtagctttaggcggttcgattttagagcgtcactttactgaccgcatggatcgcccaggtccggatattgtatgctctatgaatccggatacttttaaagagctcaagcaaggcgctcatgctttaaaattggcacgcggcggcaaaaaagacacgattatcgcgggagaaaagccaactaaagatttcgcctttgcatctgtcgtagcagataaagacattaaaaaaggagaactgttgtccggagataacctatgggttaaacgcccaggcaatggagacttcagcgtcaacgaatatgaaacattatttggtaaggtcgctgcttgcaatattcgcaaaggtgctcaaatcaaaaaaactgatattgaataagtcgactctagaggatccccgggtaccgagctcgaattcactggccgtcgttttacaacgtcgtgactgggaaaac

SEQ ID NO: 64 - DNA, 1032 bp, Drosophila melanogaster
atgagcgaatttatgccgtatgatggtcaccgccatggtgatgttaatgatgcacatcatagccacgacatgatggaaatgagcggtccggaacaggatgttggtggtcatgaacatgtgcatgaaaatagcaccattgcagaacgtctgtatagcgaagttcgtgttctgtgttggattatgaccaatccgagcaaccatcagaaaaaagcacgtcatgttaaacgtacctggggtaaacgttgcaacaaactgatttttatgagcagcgccaaagatgatgaactggatgcagttgcactgccggttggtgaaggtcgtaataatctgtggggtaaaaccaaagaggcctacaaatatatctacgaacaccatatcaacgatgccgattggtttctgaaagcagatgatgatacctataccatcgtggaaaacatgcgctatatgctgtatccgtatagtccggaaacaccggtttattttggctgtaaattcaaaccgtacgtgaaacagggttatatgagcggtggtgcaggttatgttctgagccgtgaagcagttcgtcgttttgttgttgaagcactgccgaatccgaaactgtgtaaaagcgataatagcggtgccgaagatgtggaaattggtaaatgtctgcagaatgttaatgttctggcaggcgatagccgtgatagcaatggtcgtggtcgtttttttccgtttgtgccggaacatcatctgattccgagccataccgacaaaaaattctggtattggcagtatattttctataaaaccgatgaaggcctggattgttgtagcgataatgcaatcagctttcattatgttagcccgaatcagatgtatgtgctggattatctgatctatcatctgcgtccgtatggcattattaacacaccggatgccctgccgaataaactggcagttggtgaactgatgccggaaattaaagaacaggcaaccgaaagcaccagtgatggtgttagcaaacgtagcgcagaaaccaaaacccagtaa

SEQ ID NO: 65 - DNA, 783 bp, Campylobacter jejuni
atgaaaaaagttattattgctggaaatggaccaagtttaaaagaaattgattattcaagactaccaaatgattttgatgtatttagatgtaatcaattttattttgaagataaatactatcttggtaaaaaatgcaaggcagtattttacaatcctagtcttttttttgaacaatactacactttaaaacatttaatccaaaatcaagaatatgagaccgaactaattatgtgttctaattacaaccaagctcatctagaaaatgaaaattttgtaaaaactttttacgattattttcctgatgctcatttgggatatgattttttcaaacaacttaaagattttaatgcttattttaaatttcacgaaatttatttcaatcaaagaattacctcaggggtctatatgtgtgcagtagccatagccctaggatacaaagaaatttatctttcgggaattgatttttatcaaaatgggtcatcttatgcttttgatactaaacaaaaaaatcttttaaaattggctcctaattttaaaaatgataattcacactatattggacatagtaaaaatacagatataaaagctttagaatttctagaaaaaacttacaaaataaaactatattgcttatgtcctaacagtcttttagcaaattttatagaactagcgccaaatttaaattcaaattttatcatacaagaaaaaaataactacactaaagatatactcataccttctagtgaggcttatggaaaattttcaaaaaatattaatttttaa

SEQ ID NO: 66 - DNA, 3543 bp, Artificial Sequence
  APO-3 hPDI-quescin-sulfydryl oxidase fusion protein (hPDI-QSOX1b)
atgtctgactctgaagttaaccaggaagctaaaccagaagttaaaccggaagtgaaaccggaaacccacatcaacctgaaagttagcgacggctcttctgaaatcttctttaaaattaaaaagaccactccgctgcgtcgtctgatggaagcgttcgctaaacgtcagggtaaagaaatggattctctgcgcttcctgtatgatggcatccgtattcaggctgaccaaaccccggaagatctggatatggaagataacgatatcattgaagcgcaccgtgaacaaatcggcggcatggacgcaccggaagaagaagaccacgtcctggttctgcgtaaatctaacttcgcggaagcgctggcggcgcacaaatacctgctggttgaattctacgcgccgtggtgcggtcactgcaaagctctggcgccggaatacgctaaagccgctggcaaactgaaagctgaaggttccgaaatccgcttggctaaagtagacgcaactgaagaatctgacctggcacagcagtacggtgttcgtggctacccgaccatcaaattcttccgtaacggtgacaccgctagccctaaggaatataccgcaggccgtgaagctgatgatattgttaactggctgaaaaaacgtaccggcccggcggcaaccaccctgcctgacggtgctgcggctgaatccctggttgaatcttccgaagtcgcggtaattggcttcttcaaagatgtggaatctgatagcgctaaacagttcctgcaggccgctgaagcaatcgatgatatcccgttcggtatcacctctaactctgacgtattctctaaataccagctggataaagatggcgtagttctgtttaaaaaattcgacgaaggtcgtaacaacttcgaaggtgaagtaaccaaagaaaacctgctggacttcattaaacacaaccagctgccgctggttattgaattcaccgagcagactgctcctaaaatcttcggtggcgaaattaagactcacatcctgctgttcttgccgaaaagcgtgtctgattacgacggtaaactgagcaacttcaaaaccgcggcagagtctttcaaaggtaaaatcttgttcatcttcatcgacagcgaccacactgataaccagcgtatcctggaattcttcggtctgaagaaggaagaatgtccggcggttcgcctgatcaccctggaagaagaaatgactaaatataaaccggagtctgaagaactgactgctgaacgcatcaccgaattctgccaccgctttctggaaggcaaaatcaaaccgcacctgatgagccaggaactgccagaggactgggataaacagccggttaaggtgctggttggtaaaaactttgaggatgttgcttttgacgaaaagaaaaacgtgttcgttgagttctatgcgccgtggtgcggccactgcaaacagctggcaccgatctgggacaaactcggcgagacctacaaagatcatgagaacatcgtaatcgcgaaaatggactccaccgcgaatgaagttgaagcagttaaagtgcactctttcccgaccctgaaatttttcccggcgtccgctgaccgtaccgtgattgactacaacggtgaacgcaccctggacggctttaaaaaattcctggaatccggtggtcaggatggtgctggtgatgatgacgatctggaagaccttgaagaagctgaagagccggatatggaagaagatgatgatcagaaggcggtgaaggatgaactgccgaccccgacccctaccactccgactccgaccccgactaccgctccgcgttctgcactgtacagcccatctgacccactgaccctgctgcaggcggataccgttcgtggcgccgtactgggttcccgcagcgcctgggcggttgaattcttcgcgtcctggtgtggccactgtatcgcattcgctccgacttggaaagcgctggctgaagatgttaaagcctggcgcccggcgctgtacctggcggcgctggactgcgcggaagaaactaactccgcagtgtgccgtgatttcaatatcccaggtttcccgaccgtacgcttctttaaagcatttaccaaaaacggttccggcgcagtattcccggtagcaggtgctgatgttcagactctgcgtgaacgtctgattgacgcgctggaatctcaccatgacacctggccaccggcatgtccgccgctggaacctgccaaactggaggaaattgatggcttcttcgcgcgtaacaacgaagaatatctggcactgatcttcgaaaagggcggttcttacctgggtcgcgaagtggctctggacctgagccaacacaaaggtgttgctgttcgtcgtgttctgaacactgaagcaaacgtagtgcgtaaattcggtgtgactgacttccctagctgctacctgctgtttcgtaacggttctgttagtcgtgttccggttctgatggaatcacgctccttctacactgcatatctgcagcgcctctccggtctgacccgtgaagccgcgcagaccactgttgcaccgaccaccgctaacaaaatcgcaccgaccgtttggaagctcgccgaccgcagcaagatctatatggctgacctggaatctgccttgcactacatcctgcgcattgaagtaggtcgcttcccggtactggagggccagcgcctggtcgcactgaaaaaattcgtagcggtcctggctaaatatttccctggtcgtccgctggttcagaacttcctgcacagcgttaatgaatggctgaaacgtcagaaacgtaacaaaatcccgtatagcttcttcaaaaccgcactggatgaccgtaaagaaggcgcggtcctcgctaaaaaagttaactggattggttgccagggtagcgaaccgcatttccgtggtttcccgtgctctctgtgggtcctgttccacttcctgactgtgcaggcggctcgccagaacgtggaccactctcaggaagctgcgaaagcgaaagaagtcctgccggcgatccgtggttacgttcactacttctttggctgtcgtgactgcgctagccactttgaacagatggcggccgcttccatgcaccgtgtaggttccccgaacgctgccgtgctgtggctgtggtccagccacaaccgtgttaacgctcgtctggcgggtgcaccgtctgaagatccgcagttcccgaaagttcagtggccgccgcgtgaactgtgcagcgcttgccataacgaacgcctggacgtgccggtgtgggacgttgaagctaccctgaacttcctgaaagctcacttctctccgtctaacatcatcctcgacttcccggcagcgggtagcgcggcacgtcgtgatgttcagaacgttgcggcagctccggaactggcgatgggcgccctggaactggaatcccgtaactccaccctggatccgggtaaaccggaaatgatgaaatctccgaccaacaccaccccgcacgttccggctgaaggtccggaactgatctaataa

SEQ ID NO: 67 - DNA, 39 bp, Artificial Sequence
  primer LS23-IF
gggatcggaattcgagctccacgacaggtttcccgactg

SEQ ID NO: 68 - DNA, 39 bp, Artificial Sequence
  primer LS23-VR
cagtcgggaaacctgtcgtggagctcgaattccgatccc

SEQ ID NO: 69 - DNA, 44 bp, Artificial Sequence
  primer LS23-VF
caacgtcgtgactgggaaaaccatggcgcctaacctaaactgac

SEQ ID NO: 70 - DNA, 44 bp, Artificial Sequence
  primer LS23-IR
gtcagtttaggttaggcgccatggttttcccagtcacgacgttg

SEQ ID NO: 71 - DNA, 32 bp, Artificial Sequence
  primer LS24_F
gaaggagatataccatgacaaggactagaatg

SEQ ID NO: 72 - DNA, 32 bp, Artificial Sequence
  primer LS24_R
cattctagtccttgtcatggtatatctccttc

SEQ ID NO: 73 - DNA, 60 bp, Artificial Sequence
  primer LS19-VF
tatattcaaatatataaaataaaaccgtgagaattccctgcaggtaattaaataagcttc

SEQ ID NO: 74 - DNA, 45 bp, Artificial Sequence
  primer LS19-VR
gagtgcatgtgcatgggcgcatatgtgaaatccttccctcgatcc

SEQ ID NO: 75 - DNA, 45 bp, Artificial Sequence
  primer LS19-1F
ggatcgagggaaggatttcacatatgcgcccatgcacatgcactc

SEQ ID NO: 76 - DNA, 50 bp, Artificial Sequence
  primer LS19-1R
ctcctaagcatcgatggatcttagcgacctttaaaaatgcgaatcttatt

SEQ ID NO: 77 - DNA, 50 bp, Artificial Sequence
  primer LS19-2F
aataagattcgcatttttaaaggtcgctaagatccatcgatgcttaggag

SEQ ID NO: 78 - DNA, 60 bp, Artificial Sequence
  primer LS19-2R
gaagcttatttaattacctgcagggaattctcacggttttattttatatatttgaatata

SEQ ID NO: 79 - DNA, 45 bp, Artificial Sequence
  primer LS25_VF
taaaaccgtgagaattccctgatcgatgataagctgtcaaacatg

SEQ ID NO: 80 - DNA, 44 bp, Artificial Sequence
  primer LS25_VaR_VF
catccataatacctcctgtcgactgtttccgcatgcttattaac

SEQ ID NO: 81 - DNA, 44 bp, Artificial Sequence
  primer LS25_IaF
gttaataagcatgcggaaacagtcgacaggaggtattatggatg

SEQ ID NO: 82 - DNA, 43 bp, Artificial Sequence
  primer LS25_IaR
gctcatttcagaatatttgccattatttgccgctggtcatttt

SEQ ID NO: 83 - DNA, 43 bp, Artificial Sequence
  primer LS25_IbF
aaaatgaccagcggcaaataatggcaaatattctgaaatgagc

SEQ ID NO: 84 - DNA, 45 bp, Artificial Sequence
  primer LS25_IR
catgtttgacagcttatcatcgatcagggaattctcacggtttta

SEQ ID NO: 85 - DNA, 44 bp, Artificial Sequence
  primer LS25_IbF
aattgaccaacaaggaccatagattcatatgcgcccatgcacat

SEQ ID NO: 86 - DNA, 44 bp, Artificial Sequence
  primer LS25_VbR
atgtgcatgggcgcatatgaatctatggtccttgttggtcaatt

SEQ ID NO: 87 - DNA, 51 bp, Artificial Sequence
  primer LS26_IF
attgaccaacaaggaccatagattatgtttaaaatttcaatcatcttacca

SEQ ID NO: 88 - DNA, 45 bp, Artificial Sequence
  primer LS26_IR
gctcatgtttgacagcttatcattctgcaggtcgacttatttgtt

SEQ ID NO: 89 - DNA, 45 bp, Artificial Sequence
  primer LS26_VF
aacaaataagtcgacctgcagaatgataagctgtcaaacatgagc

SEQ ID NO: 90 - DNA, 51 bp, Artificial Sequence
  primer LS26_VR
tggtaagatgattgaaattttaaacataatctatggtccttgttggtcaat

SEQ ID NO: 91 - DNA, 51 bp, Artificial Sequence
  primer LS47_VF
gattcgcatttttaaaggtcgctaaatcgatgataagctgtcaaacatgag

SEQ ID NO: 92 - DNA, 51 bp, Artificial Sequence
  primer LS47_VR
cgctcatatgtatatctccttcttaaagctagactactgctgcaggttgag

SEQ ID NO: 93 - DNA, 51 bp, Artificial Sequence
  primer LS47_1F
ctcaacctgcagcagtagtctagctttaagaaggagatatacatatgagcg

SEQ ID NO: 94 - DNA, 52 bp, Artificial Sequence
  primer LS47_1R
cgctaataagaattttcataatacctccttttactgggttttggtttctgcg

SEQ ID NO: 95 - DNA, 52 bp, Artificial Sequence
  primer LS47_2F
cgcagaaaccaaaacccagtaaaaggaggtattatgaaaattcttattagcg

SEQ ID NO: 96 - DNA, 51 bp, Artificial Sequence
  primer LS47_2R
ctcatgtttgacagcttatcatcgatttagcgacctttaaaaatgcgaatc

SEQ ID NO: 97 - DNA, 53 bp, Artificial Sequence
  primer LS44-IF
ctcaacctgcagcagtagtctaggtataagaaggagatatacatatgacctgc

SEQ ID NO: 98 - DNA, 53 bp, Artificial Sequence
  primer LS44_VR
gcaggtcatatgtatatctccttcttatacctagactactgctgcaggttgag

SEQ ID NO: 99 - DNA, 52 bp, Artificial Sequence
  primer LS44_VF
gtcgagtctggtaaagaaaccgaaggaggtattatgaaaattcttattagcg

SEQ ID NO: 100 - DNA, 52 bp, Artificial Sequence
  primer LS44_IR
cgctaataagaattttcataatacctccttcggtttctttaccagactcgac

SEQ ID NO: 101 - DNA, 57 bp, Artificial Sequence
  primer LS45_IF
ctcaacctgcagcagtagtctaggtataagaaggagatatacatatgatgtctaagg

SEQ ID NO: 102 - DNA, 57 bp, Artificial Sequence
  primer LS45_VR
ccttagacatcatatgtatatctccttcttatacctagactactgctgcaggttgag

SEQ ID NO: 103 - DNA, 55 bp, Artificial Sequence
  primer LS45b_VF
gctaagtcgagtctggtaaagaaacaaggaggtattatgaaaattcttattagcg

SEQ ID NO: 104 - DNA, 55 bp, Artificial Sequence
  primer LS45b_IR
cgctaataagaattttcataatacctccttgtttctttaccagactcgacttagc

SEQ ID NO: 105 - DNA, 53 bp, Artificial Sequence
  primer LS39_IF
ataaaaccgtgagaattccctggtataagaaggagatatacatatgaaaaaag

SEQ ID NO: 106 - DNA, 53 bp, Artificial Sequence
  primer LS39_VR
cttttttcatatgtatatctccttcttataccagggaattctcacggttttat

SEQ ID NO: 107 - DNA, 55 bp, Artificial Sequence
  primer LS39_VF
caaaaaatattaatttttaagtcgagtctggatcgatgataagctgtcaaacatg

SEQ ID NO: 108 - DNA, 55 bp, Artificial Sequence
  primer LS39_IR
catgtttgacagcttatcatcgatccagactcgacttaaaaattaatattttttg